(19) United States
(12) Patent Application Publication
(10) Pub. No.: US 2010/0261252 A1
(43) Pub. Date: Oct. 14, 2010

(54) METHODS OF ENHANCING YIELD OF ACTIVE IGA PROTEASE

(75) Inventors: Shinong Long, Milpitas, CA (US); Michel C. Vellard, San Rafael, CA (US)

Correspondence Address: Marshall, Gerstein & Borun LLP (Biomarin), 233 South Wacker Drive, 6300 Willis Tower, Chicago, IL, 60606, US

(73) Assignee: BIOMARIN PHARMACEUTICAL INC., Novato, CA (US)

(21) Appl. No.: 12/757,547

(22) Filed: Apr. 9, 2010

Related U.S. Application Data
(60) Provisional application No. 61/168,429, filed on Apr. 10, 2009.
(60) Provisional application No. 61/234,004, filed on Aug. 14, 2009.

(51) Int. Cl. C12N 9/54; C12N 9/50; C12N 9/52; C12N 1/21

(52) U.S. Cl. 435/221; 435/219; 435/220; 435/252.33; 435/252.31; 435/252.35; 435/252.3

(57) ABSTRACT

The present disclosure relates in general to methods for recombinantly producing soluble, active IgA proteases (e.g., IgA1 proteases) in host cells (e.g., bacterial cells), and methods for using IgA proteases (e.g., IgA1 proteases) produced by the methods to treat IgA deposition disorders (e.g., IgA nephropathy).

### CROSS-REFERENCE TO RELATED APPLICATIONS

The present application claims the priority and benefit of U.S. Provisional Application No. 61/168,429, filed on Apr. 10, 2009, and U.S. Provisional Application No. 61/234,004, filed on Aug. 14, 2009, the disclosure of each of which is incorporated herein by reference in its entirety.

### FIELD

The present disclosure relates in general to methods for recombinant production of soluble, active immunoglobulin A (IgA) proteases (e.g., IgA1 proteases) from host cells (e.g., bacterial cells). The disclosure also relates to treatment of IgA deposition disorders (e.g., IgA nephropathy) using a recombinant soluble, active IgA protease (e.g., IgA1 protease) produced by the methods described herein.

### BACKGROUND

Immunoglobulin A (IgA) proteases from bacteria, for example, Neisseria meningitidis, Neisseria gonorrhoeae, Haemophilus influenzae, Streptococcus pneumoniae, Ureaplasma urealyticum, Clostridium ramosum, Streptococcus pneumoniae, Streptococcus infantis, Streptococcus sanguinis, Streptococcus oxalis, Streptococcus mitis, and Gemella haemolysans (Qiu et al., Infect. Immun., 64:933-937 (1996); Poulsen et al., Infect. Immun., 66:181-190 (1998); Takenouchi-Ohkubo et al., Microbiol., 152:2171-2180 (2006)) and other bacterial strains are extracellular proteases that specifically cleave the hinge region of the human IgA antibody, the predominant class of immunoglobulin present on mucosal membranes. Bacterial IgA1 proteases are specific post-proline endopeptidases that cleave human IgA1 in the hinge region (Plaut et al., Annu. Rev. Microbiol., 37:603-622 (1983); Kilian et al., APMIS, 104:321-338 (1996)). Certain IgA proteases also cleave IgA2 and secretory IgA (sIgA) antibodies. The bacterial IgA protease is able to cleave human IgA antibody in vivo and is thought to be a means by which bacteria evade the human immune system.

IgA proteases are comprised of at least two distinct families having different structural forms, including IgA-specific metalloproteinases and IgA-specific serine endopeptidases. IgA-specific metalloproteinases comprise a signal sequence and propeptide which aids in anchoring the peptide to the cell wall, and contain several sites for metal ion (e.g., zinc) binding in the protease domain (Bender et al., Mol. Microbiol., 61:526-543 (2006)). IgA-specific serine endopeptidases are expressed as a precursor protein comprising a signal peptide, an IgA protease proteolytic domain (also known as the protease domain) and a C-terminal portion consisting of two separable domains, the α protein (or a domain) and β-core domain (or (3 domain). The C-terminal β-core domain targets the protein to the cell surface membrane and facilitates secretion of an α protein-proteolytic domain polypeptide. The β domain is cleaved from the α protein and remains associated with the cell membrane (Poulsen et al., Infect. Immun., 57:3097-4105 (1989)). The α protein is also cleaved from the precursor polypeptide, leaving the protease domain as the mature protease. The IgA-specific serine endopeptidase precursor protein has a molecular weight of approximately 169 kDa, while the mature cleavage product has a molecular weight of approximately 109 kDa.

IgA nephropathy (IgAN), a disease characterized by deposition of the IgA antibody in the glomerulus, can lead to kidney dysfunction and, in certain cases, kidney failure. Exogenous proteolytic enzymes have been tested as therapy to treat IgA1 deposition in animal models (Gesualdo et al, J. Clin. Invest., 86:715-722 (1990); Nakazawa et al., J. Exp. Med., 164:1973-1987 (1986)) in an attempt to remove or destroy IgA deposits in the kidneys. The administered proteases, chymopapain and subtilisin, act by proteolytic cleavage of IgA1 deposits in the kidney, but are not specific for IgA1 molecules and digest a variety of other proteins. U.S. Pat. No. 7,407,653 and Lamm et al. (Am. J. Pathol., 172:31-36 (2008)) disclose use of isolated H. influenzae IgA1 protease to treat IgAN in animal models.

The amount of IgA protease recoverable from H. influenzae is low compared to production of other recombinant proteins, yielding approximately 0.3 mg/L. Further, H. influenzae is a pathogenic bacteria that requires hemin for growth, making it impractical for large-scale production of recombinant IgA proteases. It has been reported that IgA proteases are capable of being expressed in E. coli as inclusion bodies (U.S. Pat. No. 5,965,424), but are not produced as soluble proteins, and the total amount of protein produced is not a high yield. Additional attempts at producing IgA proteases recombinantly have resulted in IgA proteases with reduced activity, no activity, or in low yield of recombinant material recovered (see, e.g., Khomenkov et al., Mol. Genetics, Microbiol. and Virol., 22:34-40 (2007); Grundy et al., J. Bacteriol., 169:4442-50 (1987); U.S. Pat. No. 5,965,424; and Vitovski et al., Infect. Immun., 75:2875-85 (2007)).

The present disclosure provides methods of producing recombinant soluble, active IgA protease, by direct production and/or indirect production via inclusion bodies, wherein the yields of soluble recombinant IgA protease and total recombinant IgA protease protein recovered are significantly increased compared to previous methods.

### SUMMARY

The present disclosure relates to methods for improving the yield of recombinant soluble, active IgA protease polypeptides [e.g., IgA-specific serine endopeptidases (also referred to herein as “serine-type IgA proteases”)] from recombinant host cells (e.g., bacterial cells). In certain embodiments, the present methods involve expression of only a portion of an IgA protease (e.g., only the proteolytic protease domain, and neither the α protein domain nor the β-core domain), and provide increased yield of soluble, active IgA protease and increased yield of active IgA protease formed from solubilization and refolding of IgA protease inclusion bodies.

In some embodiments, the disclosure provides a host cell (e.g., a bacterial host cell) comprising a vector, the vector comprising a polynucleotide encoding a serine-type IgA protease polypeptide that comprises an IgA protease proteolytic domain and lacks an α protein domain and a β-core domain, wherein the IgA protease polypeptide is expressed from the host cell as inclusion bodies, or as a soluble polypeptide that exhibits IgA protease activity, or a combination thereof.

In other embodiments, the disclosure provides a composition comprising at least 50 grams or 75 grams wet weight of the host cells expressing an IgA protease as described herein. In certain embodiments, the wet weight is at least 75, 80, 85, 90, 95, 100, 125, 150, 200, 250, 300, 350, 400, 450, 500, 550, 600, 650, 700, 750, 800, 850, 900, 950 or 1000 grams or more of the host cells expressing an IgA protease as described herein.

In further embodiments, the disclosure provides a method for producing a serine-type IgA protease from a host cell, comprising growing a host cell comprising a vector, the vector comprising a polynucleotide encoding an IgA protease polypeptide that comprises an IgA protease proteolytic domain and lacks an α protein domain and a β-core domain, under conditions that result in expression of the IgA protease polypeptide as inclusion bodies, or as a soluble polypeptide that exhibits IgA protease activity, or a combination thereof. In certain embodiments, the method further comprises isolating the inclusion bodies, solubilizing the isolated inclusion bodies, and refolding the solubilized inclusion bodies into soluble, active IgA protease. In other embodiments, the method further comprises isolating the soluble, active IgA protease polypeptide. In yet other embodiments, the host cell is transformed with the vector prior to growing the host cell. The method can be carried out using the host cells or compositions described herein.

In some embodiments, the IgA protease polypeptides expressed or produced according to the methods described herein lack at least about 40%, 50%, 60%, 70%, 80%, 90%, 95% or 100% of the α protein domain, or lack at least about 40%, 50%, 60%, 70%, 80%, 90%, 95% or 100% of the β-core domain, or a combination thereof. All possible combinations of the aforementioned percentages of the α protein domain and β-core domain are contemplated, e.g., an IgA protease polypeptide lacking at least about 50% of the α protein domain and at least about 60% of the β-core domain, or lacking at least about 80% of the α protein domain and at least about 90% of the β-core domain, or lacking at least about 90% of the α protein domain and at least about 80% of the β-core domain, or lacking at least about 90% of the α protein domain and at least about 90% of the β-core domain. In certain embodiments, the IgA protease polypeptides expressed or produced according to the present methods lack 100% of the α protein domain and 100% of the β-core domain. In further embodiments, the IgA protease polypeptides comprise amino acids from a heterologous polypeptide.

In additional embodiments, the culturing of the host cell according to the methods described herein results in at least about 20-40 mg/L of soluble, active IgA protease. In some embodiments, the culturing of the host cell results in soluble protease productivity level (mg of soluble protease per liter of culture medium) of at least about 10, 20, 30, 40, 50, 60, 70, 80, 90, 100, 110, 120, 130, 140, 150, 160, 170, 180, 190, 200, 210, 220, 230, 240, 250, 300, 350, 400, 450 or 500 mg/L or higher. Ranges encompassing any and all of these productivity level values are contemplated, e.g., about 20-40 mg/L, about 20-50 mg/L, about 20-70 mg/L, about 20-100 mg/L or about 20-200 mg/L of soluble, active IgA protease.

In further embodiments, the methods described herein result in at least about 1-2 g/L of soluble, active IgA protease from at least about 10-20 g/L of IgA protease inclusion bodies.

In other embodiments, the host cell is grown in a volume of culture media of at least about 10 liters or 50 liters. In certain embodiments, the culture is at least about 10, 25, 50, 75 or 100 liters of culture medium. In some embodiments, the methods involve growing host cells in a volume of at least about 10, 20, 30, 40, 50, 60, 70, 80, 90, 100, 125, 150, 175, 200, 250, 300, 350, 400, 450, 500, 600, 700, 800, 900, 1000, 1500, 2000, 2500, 3000, 3500, 4000, 4500, 5000, 5500, 6000, 6500, 7000, 7500, 8000, 9,000 or 10,000 or more liters of culture medium.

In further embodiments, the expression of IgA protease results in a ratio of mg soluble, active IgA protease produced to mg total IgA protease produced of at least about 0.5% or at least about 1%.

In other embodiments, the growing of the host cell comprising the vector results in at least about a 10-fold, 50-fold or 100-fold higher production of soluble, active IgA protease, by direct production or indirect production via inclusion bodies, or both, compared to culturing under the same conditions a host cell comprising a vector that includes the entirety of the alpha and beta domains. In certain embodiments, the expression of IgA protease in host cells results in at least about 100% to about 1000%, including at least about 100%, 200%, 300%, 400%, 500%, 600%, 700%, 800%, 900% or 1000%, increased yield, or at least about 1000% to about 10,000%, including at least about 1000%, 2000%, 3000%, 4000%, 5000%, 6000%, 7000%, 8000%, 9000% or 10,000%, increased yield of soluble and active IgA protease, as compared to recombinant production of an IgA protease comprising the full-length serine-type protease sequence.

In some embodiments, the host cells are cultured at, and methods of the disclosure are carried out at, total protein productivity level (mg or grams of total IgA protease, including soluble and insoluble, per liter of culture medium) of at least about 20, 40, 60, 80, 100, 150, 200, 250, 300, 350, 400, 450, 500, 550, 600, 650, 700, 750, 800, 850, 900, 950 or 1000 mg/L, or 2, 4, 6, 8, 10, 15, 20, 30, 40, 50, 60, 70, 80, 90 or 100 g/L.

In additional embodiments, the amount of active protein produced or isolated is, e.g., at least about 10, 25, 50, 75 or 100 grams of active IgA protease, optionally combined with a pharmaceutically acceptable carrier, excipient or diluent or a sterile pharmaceutically acceptable carrier, excipient or diluent. In certain embodiments, the amount of active protein produced or isolated is at least about 10, 20, 30, 40, 50, 60, 70, 80, 90, 100, 125, 150, 175, 200, 300, 400, 500, 600, 700, 800, 900, 1000 or more grams of active IgA protease.

In some embodiments, the IgA protease produced by the methods described herein is a bacterial IgA protease. In certain embodiments, the bacterial IgA protease is selected from the group consisting of Haemophilus influenza IgA proteases, Neisseria gonorrhoeae IgA proteases, and Neisseria meningitidis IgA proteases. In further embodiments, the IgA protease produced by the methods described herein is an IgA1 protease. In certain embodiments, the IgA1 protease is a bacterial IgA1 protease. In additional embodiments, the IgA protease produced by the methods described herein is at least about 40%, 45%, 50, 55%, 60%, 65%, 70%, 75%, 80%, 85%, 90%, 95%, 96%, 97%, 98%, 99% or 100% identical to the IgA protease set forth in SEQ ID NO: 1, 2, 3, 4, 5, 6, 7, 8, 9, 10, 11, 12, 22 or 23.

In some embodiments, the host cell is a bacterial host cell. In certain embodiments, the bacterial host cell is selected from the group consisting of E. coli, Bacillus, Streptomyces, and Salmonella. In certain embodiments, the E. coli cell is selected from the group consisting of BL21(DE3), BL21(DE3)pLysS, BL21(DE3)pGro7, ArcticExpress, ArcticExpress(DE3), C41(DE3) (or C41), C43(DE3) (or C43), Origami B, Origami B(DE3), Origami B(DE3)pLysS, KRX, and Tuner(DE3).

In additional embodiments, the host cell is grown for a time period at a temperature from about 10° C. to about 30° C., or from about 10° C. to about 40° C. In certain embodiments, the host cell is grown for a time period at about 10° C., 12° C., 15° C., 20° C., 22° C., 25° C., 27° C., 28° C., 30° C., 35° C., 37° C. or 40° C. In certain embodiments, the host cell is grown for a time period at about 28° C. or 37° C.

In further embodiments, the expression of the polynucleotide is enhanced using an isopropyl β-D-1-thiogalactopyranoside (IPTG)-inducible vector. In some embodiments, the host cell is grown at a temperature from about 10° C. to about 30° C., or from about 10° C. to about 40° C., when cultured with IPTG. In certain embodiments, the host cell is grown at about 10° C., 12° C., 15° C., 20° C., 22° C., 25° C., 27° C., 28° C., 30° C., 35° C., 37° C. or 40° C. when cultured with IPTG. In certain embodiments, the host cell is grown at about 28° C. or 37° C. when cultured with IPTG.

In still further embodiments, the IPTG is at a concentration from about 0.2 mM to about 1 mM, or from about 0.2 mM to about 2 mM. In some embodiments, the IPTG is at a concentration from about 0.4 to about 0.6 mM, or from about 0.4 mM to about 1 mM. In certain embodiments, the IPTG is at a concentration of about 0.2, 0.3, 0.4, 0.5, 0.6, 0.7, 0.8, 0.9, 1, 1.1, 1.2, 1.3, 1.4, 1.5, 1.6, 1.7, 1.8, 1.9 or 2 mM. In certain embodiments, the IPTG is at a concentration of about 0.4 mM or about 1 mM.

In other embodiments, the vector is a plasmid. In certain embodiments, the plasmid is selected from the group consisting of pET21a, pColdIV, pJexpress401, PHT01, pHT43, and pIBEX. In additional embodiments, the plasmid comprises a promoter. In certain embodiments, the promoter is selected from the group consisting of a T7 promoter, a T5 promoter, a cold shock promoter, and a pTAC promoter.

In further embodiments, the polynucleotide further encodes a signal peptide. In certain embodiments, the signal peptide is an IgA protease signal peptide. In other embodiments, the signal peptide is a heterologous signal peptide.

Additional embodiments relate to a composition comprising an active IgA protease (e.g., an IgA1 protease) produced according to the methods described herein, or a host cell described herein. In some embodiments, the composition is a pharmaceutical composition that comprises one or more pharmaceutically acceptable carriers, excipients and/or diluents.

In certain embodiments, the composition comprises an IgA protease (e.g., an IgA1 protease) that is at least about 90%, 91%, 92%, 93%, 94%, 95%, 96%, 97%, 98%, 99% or 99.5% pure, optionally as determined by any of the analytical techniques known in the art, including without limitation SDS-PAGE, Coomassie blue staining, silver staining, size-exclusion chromatography, and reverse-phase HPLC.

In some embodiments, the composition comprises an IgA1 protease that is at least about 70%, 75%, 80%, 85%, 90%, 95% or 100% identical to any one of SEQ ID NOs:1-12, 22 and 23. In certain embodiments, the IgA1 protease comprises a proteolytic protease domain that is at least about 70%, 75%, 80%, 85%, 90%, 95% or 100% identical to the proteolytic protease domain of the IgA1 protease polypeptide having the amino acid sequence of any one of SEQ ID NOs:1-12, 22 and 23.

In some embodiments, the composition is a sterile composition comprising (1) an active serine-type IgA protease (e.g., an IgA1 protease) that contains a proteolytic protease domain and lacks an α protein domain and a β-core domain, as described herein, and (2) one or more pharmaceutically acceptable excipients, diluents and/or carriers. In certain embodiments, the sterile composition is administered to a subject for treating or preventing any of the IgA deposition disorders disclosed herein.

In other embodiments, the composition is in a liquid form (e.g., an aqueous solution), or in a solid form (e.g., a lyophilized powder) that can be reconstituted in liquid form (e.g., an aqueous solution). In some embodiments, the composition is in a liquid form (e.g., an aqueous solution) or a solid form (e.g., a lyophilized powder) that is stable at a refridgerator temperature (e.g., about 5° C. or colder) or room temperature for at least about 3, 6, 9, 12, 15, 18, 21 or 24 months. In certain embodiments, a composition is stable if it retains at least about 90%, 91%, 92%, 93%, 94%, 95%, 96%, 97%, 98% or 99% of the initial amount of the IgA protease (e.g., an IgA1 protease) over the time period and under the conditions of storage.

Further embodiments relate to a method of treating or preventing a condition or disorder associated with IgA deposition, comprising administering to a subject an IgA protease (e.g., an IgA1 protease) produced according to the methods described herein or by a host cell described herein. In certain embodiments, the condition or disorder is selected from the group consisting of IgA nephropathy, hematuria, dermatitis herpetiformis, Henoch-Schoenlein purpura, Berger's disease, renal failure, liver disease, celiac disease, rheumatoid arthritis, Reiter's disease, ankylosing spondylitis, linear IgA disease, and HIV disorders (e.g., AIDS).

In additional embodiments, the present disclosure provides IgA proteases (e.g., IgA1 proteases) and compositions comprising an IgA protease (e.g., an IgA1 protease) for use in the treatment or prevention of a condition or disorder associated with IgA deposition. In related embodiments, the disclosure provides use of an IgA protease (e.g., an IgA1 protease), or a composition comprising an IgA protease (e.g., an IgA1 protease), in the manufacture of a medicament for the treatment or prevention of an IgA deposition disorder.

Other features and advantages of the disclosure will become apparent from the following detailed description. It should be understood, however, that the detailed description and specific examples are given by way of illustration only, as various changes and modifications within the spirit and scope of the disclosure will become apparent to those skilled in the art from the detailed description and examples.

### BRIEF DESCRIPTION OF THE DRAWINGS

FIG. 1 illustrates IgA1 protease expression constructs used in the present methods. L: a signal peptide to target it to the periplasm; a, b, c: three IgA protease self-cleavage sites. The IgA protease proteolytic domain with or without the signal peptide was cloned into pET21a and pColdIV expression vectors.

FIG. 2 shows that pET-S-IGAN and pET-IGAN proteases were expressed in E. coli as inclusion bodies. The expression of pET-S-IGAN and pET-IGAN was induced in BL21(DE3) cells with 1 mM IPTG at 30° C. for 3 hours. M: protein marker; 1: uninduced pET-S-IGAN clone #1 cell lysate; 2: uninduced pET-S-IGAN clone #1 soluble supernatant; 3: IPTG induced pET-S-IGAN clone #1 cell lysate; 4: IPTG induced pET-S-IGAN clone #1 soluble supernatant; 5: IPTG induced pET-S-IGAN clone #2 cell lysate; 6: IPTG induced pET-S-IGAN clone #2 soluble supernatant; 7: IPTG induced pET-IGAN clone #1 cell lysate; 8: IPTG induced pET-IGAN clone #1 soluble supernatant; 9: IPTG induced pET-IGAN clone #2 cell lysate; 10: IPTG induced pET-IGAN clone #2 soluble supernatant.

FIG. 3 depicts soluble fractions of expressed pET-S-IGAN protease isolated from E. coli. The expression of pET-S-IGAN was induced at low temperature (12° C.) and low amount of IPTG (0.4 mM) in different cell strains. M: protein marker; 1: uninduced BL21(DE3) cell lysate; 2: uninduced BL21(DE3) soluble supernatant; lanes 3-14: IPTG induced; 3: BL21(DE3) cell lysate; 4: BL21(DE3) soluble supernatant; 5: C41(DE3) cell lysate; 6: C41(DE3) soluble supernatant; 7: C43(DE3) cell lysate; 8: C43(DE3) soluble supernatant; 9: BL21(DE3)pGro7 cell lysate; 10: BL21(DE3)pGro7 soluble supernatant; 11: Origami B(DE3) cell lysate; 12: Origami B(DE3) soluble supernatant; 13: Origami B(DE3)pLysS cell lysate; 14: Origami B(DE3)pLysS soluble supernatant.

FIG. 4 depicts soluble expressed pCold-IGAN protease isolated from E. coli. The expression of pCold-IGAN was induced at low temperature (15° C.) and low amount of IPTG (0.4 mM) in different cell strains. M: protein marker; 1: uninduced BL21(DE3) cell lysate; 2: uninduced BL21(DE3) soluble supernatant; lanes 3-14: IPTG induced; 3: BL21(DE3) cell lysate; 4: BL21(DE3) soluble supernatant; 5: C41(DE3) cell lysate; 6: C41(DE3) soluble supernatant; 7: C43(DE3) cell lysate; 8: C43(DE3) soluble supernatant; 9: BL21(DE3)pLysS cell lysate; 10: BL21(DE3)pLysS soluble supernatant; 11: Origami B(DE3) cell lysate; 12: Origami B(DE3) soluble supernatant; 13: BL21(DE3)pGro7 cell lysate; 14: BL21(DE3)pGro7 soluble supernatant.

FIG. 5 shows that expressed soluble IgA1 proteases exhibit IgA1 cleavage activity as assessed in an IgA1 cleavage assay. IgA1 was incubated with cell lysates or soluble supernatant at 37° C. overnight. SDS-PAGE and Western blot with anti-IgA antibody (Ab) were employed to detect IgA1 cleavage. M: protein marker; 1: IgA1; 2: IgA1+B-PER lysis buffer; 3: IgA1+IgA1 protease from H. influenzae; For lanes 4 to 22, IgA1 was cleaved by IgA1 protease in supernatant of cell lysates (in crude extract for lanes 7 and 9) after expression induced with 0.4 mM IPTG at 12° C.; 4: BL21(DE3) cell lysate; 5: BL21(DE3) cell lysate+IgA1 protease; 6: NP-PAL (BLR cell) cell lysate; 7: pET-S-IGAN BL21(DE3) cell lysate induced at 37° C.; 8: pET-S-IGAN BL21(DE3) soluble supernatant induced at 37° C.; 9: pET-IGAN BL21(DE3) cell lysate induced at 37° C.; 10: pET-S-IGAN BL21(DE3) soluble supernatant induced at 37° C.; 11: pET-S-IGAN BL21(DE3) soluble supernatant; 12: pET-IGAN BL21(DE3) soluble supernatant; 13: pET-S-IGAN C41(DE3) soluble supernatant; 14: pET-IGAN C41(DE3) soluble supernatant; 15: pET-S-IGAN BL21(DE3)pGro7 soluble supernatant; 16: pET-IGAN BL21(DE3)pGro7 soluble supernatant; 17: pET-S-IGAN Origami B(DE3) soluble supernatant; 18: pET-IGAN Origami B(DE3) soluble supernatant; 19: pCold-S-IGAN BL21(DE3) soluble supernatant; 20: pCold-IGAN BL21(DE3) soluble supernatant; 21: pCold-S-IGAN Origami B soluble supernatant; 22: pCold-IGAN Origami B soluble supernatant.

FIG. 6 illustrates screening for the expression of soluble His-tagged IgA1 proteases by ELISA using anti-His antibody. The expression of the IgA1 protease-expressing constructs in the following cell strains was induced with 0.4 mM IPTG at 15° C. The cell pellets were lysed and centrifuged, and the resulting soluble supernatants were screened using ELISA with anti-His antibody. 1: negative control, BL21(DE3) cell lysate; pET-IGAN (#2-#10) in 2: BL21(DE3); 3: Tuner(DE3); 4: C43(DE3); 5: Origami B(DE3); 6: Origami B(DE3)pLysS; 7: KRX; 8: ArcticExpress(DE3); 9: BL21(DE3)pGro7; 10: C41(DE3); pET-S-IGAN (#11-#18) in 11: BL21(DE3); 12: C41(DE3); 13: C43(DE3); 14: Origami B(DE3); 15: KRX; 16 ArcticExpress(DE3); 17: BL21(DE3)pGro7; 18: Tuner(DE3); pCold-S-IGAN (#19-#23) in 19: BL21(DE3); 20: C41(DE3); 21: C43(DE3); 22: Origami B; 23: BL21(DE3)pGro7; pCold-IGAN (#24-#28) in 24: BL21(DE3); 25: C41(DE3); 26: C43(DE3); 27: Origami B; 28: BL21(DE3)pGro7.

FIG. 7 illustrates screening for the expression of soluble His-tagged IgA1 protease from pET-IGAN by ELISA using anti-His antibody. The expression of pET-IGAN was induced in six cell strains with 0.4 mM or 1 mM IPTG at 12° C. or 20° C. P: 1 ug purified IgA1 protease from H. influenzae in BL21(DE3) soluble supernatant; N: negative control, BL21(DE3) soluble supernatant; BL21(DE3)pGro7 (#1-#4) soluble supernatant induced at 1: 0.4 mM IPTG, 12° C.; 2: 1.0 mM IPTG, 12° C.; 3: 0.4 mM IPTG, 20° C.; 4: 1.0 mM IPTG, 20° C.; ArcticExpress(DE3) (#5-#8) soluble supernatant induced at 5: 0.4 mM IPTG, 12° C.; 6: 1.0 mM IPTG, 12° C.; 7: 0.4 mM IPTG, 20° C.; 8: 1.0 mM IPTG, 20° C.; Origami B(DE3) (#9-#12) soluble supernatant induced at 9: 0.4 mM IPTG, 12° C.; 10: 1.0 mM IPTG, 12° C.; 11: 0.4 mM IPTG, 20° C.; 12: 1.0 mM IPTG, 20° C.; BL21(DE3) (#13-#16) soluble supernatant induced at 13: 0.4 mM IPTG, 12° C.; 14: 1.0 mM IPTG, 12° C.; 15: 0.4 mM IPTG, 20° C.; 16: 1.0 mM IPTG, 20° C.; C41(DE3) (#17-#20) soluble supernatant induced at 17: 0.4 mM IPTG, 12° C.; 18: 1.0 mM IPTG, 12° C.; 19: 0.4 mM IPTG, 20° C.; 20: 1.0 mM IPTG, 20° C.; Tuner(DE3) (#21-#24) soluble supernatant induced at 21: 0.4 mM IPTG, 12° C.; 22: 1.0 mM IPTG, 12° C.; 23: 0.4 mM IPTG, 20° C.; 24: 1.0 mM IPTG, 20 C.

FIG. 8 shows an ELISA screen for expression of soluble His-tagged IgA1 protease from pET-IGAN in C41(DE3) E. coli strain. The expression of pET-IGAN in the C41(DE3) strain was induced at different temperatures and different concentrations of IPTG. N: uninduced pET-IGAN C41(DE3) soluble supernatant; P: 1 ug purified IgA1 protease from H. influenzae in uninduced pET-IGAN C41(DE3) soluble supernatant; pET-IGAN C41(DE3) soluble supernatant induced at 1: 15° C., 0.2 mM IPTG; 2: 15° C., 0.4 mM IPTG; 3: 15° C., 0.6 mM IPTG; 4: 20° C., 0.2 mM IPTG; 5: 20° C., 0.4 mM IPTG; 6: 20° C., 0.6 mM IPTG; 7: 26° C., 0.2 mM IPTG; 8: 26° C., 0.4 mM IPTG; 9: 26° C., 0.6 mM IPTG.

FIG. 9 shows expression of soluble His-tagged IgA1 protease from pET-IGAN in C41(DE3) E. coli cells. Western blot with anti-His Ab was employed to confirm expression of the IgA1 protease. U: uninduced pET-IGAN C41(DE3) soluble supernatant; P: 1 ug purified IgA1 protease from H. influenzae; pET-IGAN C41(DE3) soluble supernatant induced at 1: 20° C., 0.2 mM IPTG; 2: 20° C., 0.4 mM IPTG; 3: 20° C., 0.6 mM IPTG; 4: 15° C., 0.2 mM IPTG; 5: 15° C., 0.4 mM IPTG; 6: 15° C., 0.6 mM IPTG; 7: 26° C., 0.2 mM IPTG; 8: 26° C., 0.4 mM IPTG; 9: 26° C., 0.6 mM IPTG.

FIG. 10 depicts the results of an IgA1 cleavage assay of IgA1 protease produced from C41(DE3) cells containing the pET-IGAN plasmid. IgA1 antibodies were incubated with the following samples at 37° C. overnight; M: protein marker; 1: Purified IgA1 protease from H. influenzae (resulted in cleavage of IgA1); 2: PBS buffer (no cleavage of IgA1); 3: pET-IGAN-expressed IgA1 protease purified from C41(DE3) soluble supernatant (resulted in cleavage of IgA1).

FIG. 11 shows the amino acid sequence of an H. influenzae IgA1 protease having a C-terminal hexa-histidine tag expressed by constructs described herein (SEQ ID NO: 22).

FIG. 12 displays an SDS-PAGE gel of eluate fractions (“F” denotes fraction) from S300 Sephacryl column chromatography of soluble IgA1 protease produced in E. coli C41(DE3) cells. Fractions 23 and 24 were collected as the final product.

FIG. 13 shows the expression of IgA1 protease inclusion bodies in E. coli BL21(DE3) cells. M: protein marker; Tu: total un-induced cell lysate; Su: un-induced soluble supernatant; T: total induced cell lysate; S: induced soluble supernatant.

FIG. 14 displays the results of purification of refolded IgA1 protease using a Ni-NTA column. M: protein marker; 1: refolded IgA1 protease in binding buffer (50 mM Tris, 150 mM NaCl, pH 7.9); 2: flow-through fraction; 3: wash fraction; 4-11: eluted fractions.

FIG. 15 depicts the results of refolding of solubilized IgA1 protease inclusion bodies and purification of refolded IgA1 protease on an IMAC column. M: protein marker; IB: partially purified inclusion bodies in 6 M guanidine hydrochloride; F: flow-through fraction from the IMAC column; W: wash with 6 M guanidine hydrochloride and 20 mM imidazole; RF: flow-through of gradient wash of buffers for refolding; E: refolded IgA1 protease eluted off the column using increasing concentrations of imidazole; AE: IgA1 protease aggregates eluted off the column using 6 M guanidine hydrochloride and 250 mM imidazole.

FIG. 16 illustrates the identification of properly refolded IgA1 protease by HPLC-SEC (size-exclusion chromatography) and assay of IgA1 cleavage activity using an Experion automated electrophoresis system. A: HPLC-SEC chromatograph of purified soluble, active IgA1 protease having a retention time around 12.5 min (standard control). B: HPLC-SEC chromatograph of solubilized IgA1 protease inclusion bodies refolded in a particular refolding buffer. C: HPLC-SEC analysis of refolded IgA1 proteases formed in different refolding buffers 1 to 10 and having a peak height at a retention time of about 12.5 min. D: Experion virtual gel of IgA1 electropherogram—IgA1 cleavage assay of refolded IgA1 proteases formed in different refolding buffers 1 to 10 (same samples as in HPLC-SEC (C)). E: Calculated IgA1 cleavage activity, in the Experion assay (D), of refolded IgA1 proteases formed in different refolding buffers 1 to 10.

FIG. 17 relates to evaluation of human IgA1 cleavage activity of purified refolded IgA1 protease using an Experion automated electrophoresis system. A: virtual gel of IgA1 electropherogram; L: protein ladder; 1: 1600 ng/uL IgA1; 2: 400 ng/uL IgA1; 3: 100 ng/uL IgA1; 4: 25 ng/uL IgA1; 5: 0 ng/uL IgA1; 6: 9 uL of 1600 ng/uL IgA1+1 uL of 80 ng/uL IgA1 protease at 0 minute; 7: 9 uL of 1600 ng/uL IgA1+1 uL of 80 ng/uL IgA1 protease at 1 minute; 8: 9 uL of 1600 ng/uL IgA1+1 uL of 80 ng/uL IgA1 protease at 2 minutes; 9: 9 uL of 1600 ng/uL IgA1+1 uL of 80 ng/uL IgA1 protease at 3 minutes; 10: 9 uL of 1600 ng/uL IgA1+1 uL of 80 ng/uL IgA1 protease at 10 minutes. B: standard curve of human IgA1 based on lanes 1-5 in A (IgA1 concentrations of 1600, 400, 100, 25 and 0 ng/uL). C: IgA1 cleavage activity of purified refolded IgA1 protease based on decreasing concentrations of uncleaved human IgA1 calculated from lanes 6-10 in A and the standard curve of IgA1 in B.

FIG. 18 compares the purity and human IgA1 cleavage activity of three purified IgA1 proteases—soluble IgA1 protease directly produced from H. influenzae, soluble IgA1 protease directly produced from E. coli C41(DE3) cells, and refolded IgA1 protease prepared from inclusion bodies expressed in E. coli BL21(DE3) cells. A: SDS-PAGE and Coomassie blue staining of the three purified IgA1 proteases; M: protein marker; 1: soluble IgA1 protease from H. influenzae; 2: soluble IgA1 protease from C41(DE3); 3: refolded IgA1 protease from BL21(DE3). B: Human IgA1 cleavage activity of the three purified IgA1 proteases in the Experion assay; from left to right: soluble IgA1 protease from H. influenzae, soluble IgA1 protease from C41(DE3), and refolded IgA1 protease from BL21(DE3). C: HPLC-SEC analysis of the three purified IgA1 proteases; 1: soluble IgA1 protease from H. influenzae; 2: soluble IgA1 protease from C41(DE3); 3: refolded IgA1 protease from BL21(DE3).

FIG. 19 shows the results of purification of refolded IgA1 protease using an 5300 Sephacryl size-exclusion column.

### DETAILED DESCRIPTION

The present disclosure describes novel methods of recombinantly producing soluble, active IgA proteases (e.g., IgA-specific serine endopeptidases) in host cell culture (e.g., bacterial culture). In certain embodiments, the methods recombinantly produce soluble, active IgA-specific serine endopeptidases (also referred to herein as “serine-type IgA proteases”) by culturing a host cell comprising a vector that encodes an IgA protease polypeptide containing the proteolytic protease domain of the enzyme and lacking a significant portion, including the entire portion, of the α protein domain and/or β-core domain, which are part of the enzyme precursor protein. The methods provided herein result in increased yield of soluble, active IgA protease isolated from cell cytoplasmic and periplasmic locations, as well as increased yield of soluble, active IgA protease formed from solubilization and refolding of inclusion bodies. For example, the present methods produce, directly and/or indirectly via inclusion bodies, a large quantity of soluble, active H. influenzae IgA1 protease through expression of only the proteolytic protease domain. The IgA (e.g., IgA1) proteases produced by the present methods and host cells can be purified and are useful for treating disorders associated with aberrant deposition of IgA (e.g., IgA1) antibodies.

### Definitions

Unless otherwise defined, all technical and scientific terms used herein have the same meaning as commonly understood by one of ordinary skill in the art to which the present disclosure belongs. The following references provide one of skill with a general definition of many of the terms used in this disclosure: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

As used in the present disclosure and the appended claims, the terms “a”, “an” and “the” include plural reference as well as singular reference unless the context clearly dictates otherwise.

As used herein, the following terms have the meanings ascribed to them unless specified otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5% or 0.1% of a given value or range. Whenever the term “about” or “approximately” precedes the first numerical value in a series of two or more numerical values, it is understood that the term “about” or “approximately” applies to each one of the numerical values in that series.

The terms “ambient temperature” and “room temperature” are used interchangeably herein and refer to the temperature of the surrounding environment (e.g., the room in which a reaction is conducted or a composition is stored). In certain embodiments, ambient temperature or room temperature is a range from about 15° C. to about 28° C., or from about 15° C. to about 25° C., or from about 20° C. to about 28° C., or from about 20° C. to about 25° C., or from about 22° C. to about 28° C., or from about 22° C. to about 25° C. In other embodiments, ambient temperature or room temperature is about 15° C., 16° C., 17° C., 18° C., 19° C., 20° C., 21° C., 22° C., 23° C., 24° C., 25° C., 26° C., 27° C. or 28° C.

“cDNA” refers to a DNA that is complementary or identical to an mRNA, in either single-stranded or double-stranded form.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′-end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence is 5′-TATAC-3′ is complementary to a polynucleotide whose sequence is 5′-GTATA-3′. A nucleotide sequence is “substantially complementary” to a reference nucleotide sequence if the sequence complementary to the subject nucleotide sequence is substantially identical to the reference nucleotide sequence.

“Conservative substitution” refers to substitution of an amino acid in a polypeptide with a functionally, structurally or chemically similar natural or unnatural amino acid. In some embodiments, the following groups each contain natural amino acids that are conservative substitutions for one another:
(1), Alanine (A) Serine (S), Threonine (T);
(2) Aspartic acid (D), Glutamic acid (E);
(3) Asparagine (N), Glutamine (Q);
(4) Arginine (R), Lysine (K);
(5) Isoleucine (I), Leucine (L), Methionine (M), Valine (V); and
(6) Phenylalanine (F), Tyrosine (Y), Tryptophan (W).

In other embodiments, the following groups each contain natural amino acids that are conservative substitutions for one another:
(1) Glycine (G), Alanine (A);
(2) Aspartic acid (D), Glutamic acid (E);
(3) Asparagine (N), Glutamine (Q);
(4) Arginine (R), Lysine (K);
(5) Isoleucine (I), Leucine (L), Methionine (M), Valine (V), Alanine (A);
(6) Phenylalanine (F), Tyrosine (Y), Tryptophan (W); and
(7) Serine (S), Threonine (T), Cysteine (C).

In further embodiments, amino acids can be grouped as set forth below:
(1) hydrophobic: Met, Ala, Val, Leu, Ile, Phe, Trp;
(2) neutral hydrophilic: Cys, Ser, Thr, Asn, Gln;
(3) acidic: Asp, Glu;
(4) basic: His, Lys, Arg;
(5) residues that influence backbone orientation: Gly, Pro; and
(6) aromatic: Trp, Tyr, Phe, His.

The term “derivative”, when used in reference to polypeptides, refers to polypeptides chemically or non-chemically modified by such techniques as, for example and without limitation, ubiquitination, labeling (e.g., with radionuclides or various enzymes), covalent polymer attachment (e.g., derivatization with polyethylene glycol (PEG)), and insertion or substitution by chemical or non-chemical synthesis of natural or unnatural amino acids (e.g., ornithine), which may or may not normally occur in human proteins. Derivative polypeptides can be generated by methods known in the art.

The term “effective amount” means a dosage sufficient to produce a desired result on a health condition, pathology, or disease of a subject or for a diagnostic purpose. The desired result may comprise a subjective or objective improvement in the recipient of the dosage. “Therapeutically effective amount” refers to that amount of an agent effective to produce the intended beneficial effect on health.

“Encoding” refers to the inherent property of specific sequences of nucleotides in a polynucleotide, such as a gene, a cDNA or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, whose nucleotide sequence is identical to the mRNA sequence and is usually provided in sequence listings, and the non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Expression control sequence” refers to a nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (i.e., ATG), splicing signals for introns, and stop codons.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in vitro expression system. Expression vectors include all those known in the art, such as cosmids, plasmids (e.g., naked or contained in liposomes) and viruses that incorporate the recombinant polynucleotide.

The C41 E. coli cell line is also known as C41(DE3), and the C43 E. coli cell line is also known as C43(DE3).

The “protease domain”, “proteolytic domain”, “protease proteolytic domain” or “proteolytic protease domain”, used interchangeably herein, of an IgA protease refers to the domain of the IgA protease which is active in cleavage of an IgA antibody.

In some embodiments, an IgA protease polypeptide that comprises an IgA protease proteolytic domain and lacks an α protein domain and a β-core domain is an IgA-specific serine endopeptidase polypeptide that lacks a sufficient amount of the α protein domain and β-core domain characteristic of a serine-type IgA protease, such that expression of the polypeptide in host cells results in at least about 100% to about 1000%, including at least about 100%, 200%, 300%, 400%, 500%, 600%, 700%, 800%, 900% or 1000%, increased yield, or at least about 1000% to about 10,000%, including at least about 1000%, 2000%, 3000%, 4000%, 5000%, 6000%, 7000%, 8000%, 9000% or 10,000%, increased yield of soluble, active IgA protease, by direct production or indirect production via inclusion bodies, or both. In certain embodiments, an IgA protease polypeptide expressed or produced according to the present methods lacks at least about 40%, 50%, 60%, 70%, 80%, 90%, 95% or 100% of the α protein domain, and lacks at least about 40%, 50%, 60%, 70%, 80%, 90%, 95% or 100% of the β-core domain. All possible combinations of the aforementioned percentages of the α protein domain and β-core domain are contemplated, e.g., an IgA protease polypeptide lacking at least about 50% of the α protein domain and at least about 60% of the β-core domain, or lacking at least about 80% of the α protein domain and at least about 90% of the β-core domain, or lacking at least about 90% of the α protein domain and at least about 80% of the β-core domain, or lacking at least about 90% of the α protein domain and at least about 90% of the β-core domain. In an embodiment, the IgA protease polypeptide lacks 100% of the α protein domain and 100% of the β-core domain.

A soluble, active IgA protease can be directly produced in soluble and active form in the cell cytoplasm and/or periplasm, or indirectly produced through solubilization of inclusion bodies and refolding of solubilized inclusion bodies to the active form of the IgA protease. As used herein, the terms “serine-type IgA protease” and “IgA-specific serine endopeptidase” are used interchangeably.

“IgA protease activity” refers to the ability of a polypeptide to cleave mammalian (including human and great ape) IgA antibodies (e.g., in the hinge region of the IgA antibody protein sequence), resulting in fragments of the IgA antibody (e.g., intact F_{ab} and F_{c} antibody domains). Non-limiting examples of IgA proteins that can be cleaved by IgA proteases include IgA1, IgA2 and secretory IgA. For example, an H. influenzae type 1 IgA1 protease cleaves the human IgA1 hinge region at the Pro-Ser bond at residued 231 and 232, whereas H. influenza type 2 IgA1 protease cleaves IgA1 at the Pro-Thr bond at residues 235 and 236. IgA cleavage sites for additional IgA proteases are described below.

The term “IgA deposition disease” or “IgA deposition disorder”, or “a condition or disorder associated with IgA deposition”, refers to a condition or disorder suffered by a subject in which IgA antibodies form complexes in vivo and the IgA complexes are deposited in tissue(s) or other site(s) (e.g., organs) in the subject, resulting in adverse effects to the subject. Exemplary IgA deposition disorders include, but are not limited to, IgA nephropathy, hematuria, dermatitis herpetiformis, Henoch-Schoenlein purpura, Berger's disease, renal failure, liver disease, celiac disease, rheumatoid arthritis, Reiter's disease (or reactive arthritis), ankylosing spondylitis, linear IgA disease, and HIV disorders (e.g., AIDS). In some embodiments, the subject is a mammal. In an embodiment, the subject is human.

The term “precursor” or “precursor form” of an IgA protease refers to the form of IgA protease that lacks certain modification(s) (e.g., internal cleavage of the protease) which normally occur, e.g., in the cytoplasm. The term “mature,” “mature form,” “processed” or “processed form” refers to the form of IgA protease that normally exists in the extracellular space. For the recombinant IgA proteases of the disclosure, the relative abundance of “precursor” or “precursor form”, and “mature,” “mature form,” “processed” or “processed form”, can be determined by subjecting the protease preparation to electrophoretic separation by SDS-PAGE under reducing conditions followed by staining with Coomassie Blue or silver, or by chromatographic separation by HPLC (e.g., C4 reverse phase) or by any other chromatographic separation, e.g., size-exclusion chromatography (SEC) and the like.

“Naturally-occurring” as applied to an object refers to the fact that the object can be found in nature. For example, a polypeptide or polynucleotide sequence which is present in an organism (including viruses) that can be isolated from a source in nature, and which has not been intentionally modified by man in the laboratory, is naturally-occurring.

A “heterologous” sequence is an amino acid or nucleotide sequence that is not naturally found in association with the amino acid or nucleotide sequence with which it is associated.

“Pharmaceutical composition” refers to a composition suitable for pharmaceutical use in a subject animal, including mammals and humans. A pharmaceutical composition comprises a pharmacologically effective amount of a therapeutic IgA protease and optionally comprises one or more pharmaceutically acceptable carriers, diluents and/or excipients. A pharmaceutical composition encompasses a composition comprising the active ingredient(s), and the inert ingredient(s) that make up the carrier, diluent and/or excipient, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing an IgA protease of the present disclosure and one or more pharmaceutically acceptable carriers, diluents and/or excipients.

“Pharmaceutically acceptable carrier, diluent or excipient” refers to any of the standard pharmaceutical carriers, diluents, buffers, and excipients, such as, for example and without limitation, a phosphate buffered saline solution, 5% aqueous solution of dextrose, and emulsions, such as an oil/water or water/oil emulsion, and various types of wetting agents and/or adjuvants. Suitable pharmaceutical carriers, diluents or excipients and formulations are described in Remington's Pharmaceutical Sciences, 19th Ed., Mack Publishing Co. (Easton, Pa. (1995)). Preferred pharmaceutical carriers, diluents or excipients depend upon various factors, including the intended mode of administration of the active agent. Typical modes of administration include, for example and without limitation, enteral (e.g., oral) administration, parenteral (e.g., subcutaneous, intramuscular, intravenous, intraperitoneal) injection, and topical, transdermal and transmucosal administration.

A “pharmaceutically acceptable salt” is a salt suitable for pharmaceutical use, including, e.g., metal salts (e.g., sodium, potassium, magnesium, calcium, etc.), salts of ammonia and organic amines, salts of mineral acids (e.g., HCl), and salts of organic acids (e.g., acetic acid).

“Polynucleotide” refers to a polymer composed of nucleotide units. Polynucleotides include naturally occurring nucleic acids, such as deoxyribonucleic acid (“DNA”) and ribonucleic acid (“RNA”), as well as nucleic acid analogs. Nucleic acid analogs include those which contain non-naturally occurring bases, nucleotides that engage in linkages with other nucleotides other than the naturally occurring phosphodiester bond, and/or bases attached through linkages other than phosphodiester bonds. Non-limiting examples of nucleotide analogs include phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, e.g., using an automated DNA synthesizer. The term “nucleic acid” typically refers to larger polynucleotides. The term “oligonucleotide” typically refers to shorter polynucleotides. In certain embodiments, an oligonucleotide contains no more than about 50 nucleotides. It will be understood that when a nucleotide sequence is represented by a DNA sequence (i.e., A, T, G, C), this also includes an RNA sequence (i.e., A, U, G, C) in which “U” replaces “T”.

“Polypeptide” refers to a polymer composed of natural and/or unnatural amino acid residues, naturally occurring structural variants thereof, and/or synthetic non-naturally occurring analogs thereof, linked via peptide bonds. Synthetic polypeptides can be synthesized, e.g., using an automated polypeptide synthesizer. The term “protein” typically refers to larger polypeptides. The term “peptide” typically refers to shorter polypeptides. Conventional notation is used herein to portray polypeptide sequences: the left-hand end of a polypeptide sequence is the amino-terminus; the right-hand end of a polypeptide sequence is the carboxyl-terminus.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Recombinant polynucleotide” refers to a polynucleotide having sequences that are not naturally joined together. An amplified or assembled recombinant polynucleotide can be included in a suitable vector, and the vector can be used to transform a suitable host cell. A host cell that comprises the recombinant polynucleotide is referred to as a “recombinant host cell”. The recombinant polynucleotide is expressed in the recombinant host cell to produce, e.g., a “recombinant polypeptide”. A recombinant polynucleotide can also serve a non-coding function (e.g., promoter, origin of replication, ribosome-binding site, etc.).

The term “hybridizing specifically to”, “specific hybridization” or “selectively hybridize to” refers to the binding, duplexing, or hybridizing of a nucleic acid molecule preferentially to a particular nucleotide sequence under stringent conditions, e.g., highly stringent conditions, when that sequence is present in a mixture of (e.g., total cellular) DNA or RNA.

The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and Northern hybridizations are sequence-dependent, and are different under different environmental parameters. An extensive guide to the hybridization of nucleic acids is found in Tijssen, Laboratory Techniques in Biochemistry and Molecular Biology—Hybridization with Nucleic Acid Probes, Part I, Chapter 2 in “Overview of principles of hybridization and the strategy of nucleic acid probe assays”, Elsevier (New York, 1993). In certain embodiments, highly stringent hybridization and wash conditions are about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. In certain embodiments, very stringent conditions are equal to the Tₘ for a particular probe.

An example of stringent hybridization conditions for hybridization of complementary nucleic acids that have more than 100 complementary residues on a filter in a Southern or Northern blot is 50% formalin with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see Sambrook et al. for a description of SSC buffer). A high stringency wash can be preceded by a low stringency wash to remove background probe signal. An example of medium stringency wash for a duplex of, e.g., more than 100 nucleotides, is 1×SSC at 45° C. for 15 minutes. An example of low stringency wash for a duplex of, e.g., more than 100 nucleotides, is 4-6×SSC at 40° C. for 15 minutes. In general, a signal to noise ratio of 2× (or higher) than that observed for an unrelated probe in the particular hybridization assay indicates detection of a specific hybridization.

A “subject” of diagnosis or treatment is a human or non-human animal, including a mammal or a primate.

In some embodiments, the term “substantially homologous” or “substantially identical” in the context of two nucleic acids or polypeptides refers to two or more sequences or subsequences that have at least about 40%, 50%, 60%, 65%, 70%, 75%, 80%, 85%, 90%, 95%, 96%, 97%, 98% or 99% nucleotide or amino acid residue identity, when compared and aligned for maximum correspondence, as measured using one of the following sequence comparison algorithms or by visual inspection. In certain embodiments, the substantial homology or identity exists over a region of the sequences that is at least about 25 residues, or at least about 50 residues, or at least about 100 residues, or at least about 150 residues in length. In an embodiment, the sequences are substantially homologous or identical over the entire length of either or both comparison biopolymers.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith and Waterman, Adv. Appl. Math., 2:482 (1981); by the homology alignment algorithm of Needleman and Wunsch, J. Mol. Biol., 48:443 (1970); by the search for similarity method of Pearson and Lipman, Proc. Natl. Acad. Sci. USA, 85:2444 (1988); by computerized implementations of these algorithms (e.g., GAP, BESTFIT, FASTA, and TFASTA in the Wisconsin Genetics Software Package, Genetics Computer Group, Madison, Wis.); or by visual inspection.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle, J. Mol. Evol., 35:351-360 (1987). The method used is similar to the method described by Higgins and Sharp, CABIOS, 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps. Another algorithm that is useful for generating multiple alignments of sequences is Clustal W (see, e.g., Thompson et al., Nucleic Acids Research, 22:4673-4680 (1994)).

Another example of an algorithm that is suitable for determining percent sequence identity and sequence similarity is the BLAST algorithm, which is described in Altschul et al., J. Mol. Biol., 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., J. Mol. Biol., 215:403-410 (1990)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, Proc. Natl. Acad. Sci. USA, 89:10915 (1989)).

In addition to calculating percent sequence identity, the BLAST algorithm also performs a statistical analysis of the similarity between two sequences (see, e.g., Karlin and Altschul, Proc. Natl. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. In certain embodiments, a nucleic acid is considered similar to a reference sequence if the smallest sum probability in a comparison of the test nucleic acid to the reference nucleic acid is less than about 0.1, or less than about 0.01, or less than about 0.001.

In some embodiments, two nucleic acid sequences or polypeptides are substantially homologous or identical if the two molecules hybridize to each other under stringent conditions, or under highly stringent conditions, as described herein.

In some embodiments, the terms “substantially pure” and “isolated” mean an object macromolecular species is the predominant macromolecular species present on a molar basis (i.e., on a molar basis, more abundant than any other individual macromolecular species in the composition), and a substantially purified fraction is a composition wherein an object macromolecular species constitutes at least about 50% on a molar basis of all macromolecular species present. In certain embodiments, a substantially pure or isolated macromolecular species constitutes at least about 50%, 60%, 70%, 75%, 80%, 85%, 90%, 91%, 92%, 93%, 94%, 95%, 96%, 97%, 98% or 99% of the macromolecular species present on a molar basis. In other embodiments, a substantially pure composition means that about 80% to 90% or more of the macromolecular species present in the composition, on a molar basis, is the macromolecular species of interest. In further embodiments, an object macromolecular species is purified to essential homogeneity (e.g., contaminant macromolecular species cannot be detected in the composition by conventional detection methods) if the composition consists essentially of a single macromolecular species (e.g., at least about 95%, 96%, 97%, 98% or 99% of the object macromolecular species on a molar basis). Solvent species, small molecules (<about 500 Daltons), stabilizers (e.g., BSA), and elemental ion species are not considered macromolecular species for purposes of these embodiments.

In certain embodiments, the IgA proteases of the disclosure are substantially pure or isolated. In some embodiments, the IgA proteases of the disclosure are substantially pure or isolated with respect to the macromolecular starting materials used in their production. In additional embodiments, a pharmaceutical composition comprises a substantially pure or isolated IgA protease admixed with one or more pharmaceutically acceptable carriers, diluents and/or excipients.

The terms “treat”, “treating” and “treatment” encompass alleviating or abrogating a condition, disorder or disease, or one or more of the symptoms associated with the condition, disorder or disease, and encompass alleviating or eradicating the cause(s) of the condition, disorder or disease itself. In certain embodiments, the terms “treat”, “treating”, and “treatment” refer to administration of a compound, a pharmaceutical composition or a pharmaceutical dosage form to a subject for the purpose of alleviating, abrogating or preventing a condition, disorder or disease, or symptom(s) associated therewith, or cause(s) thereof. In further embodiments, the term “treatment” refers to prophylactic (preventative) treatment or therapeutic treatment or diagnostic treatment.

The terms “prevent”, “preventing” and “prevention” encompass delaying and/or precluding the onset of a condition, disorder or disease, and/or its attendant symptom(s); barring a subject from acquiring a disease; and reducing a subject's risk of acquiring a condition, disorder or disease.

A “therapeutic” treatment is a treatment administered to a subject who exhibits signs or symptoms of pathology for the purpose of diminishing or eliminating those signs or symptoms. The signs or symptoms may be biochemical, cellular, histological, functional, subjective or objective. The IgA proteases of the disclosure can be given as a therapeutic treatment, a prophylactic treatment, or for diagnosis.

### IgA Protease Polypeptides

IgA proteases are secreted by many different bacterial strains and are believed to be a virulence factor that allows the bacteria to evade the mucosal immune system. IgA proteases consist of several different families of proteins, including the IgA-specific serine endopeptidases and the IgA-specific metalloproteinases, all of which cleave IgA antibodies in the antibody hinge region, resulting in release of intact IgA F_{ab} and F_{c} antibody domains.

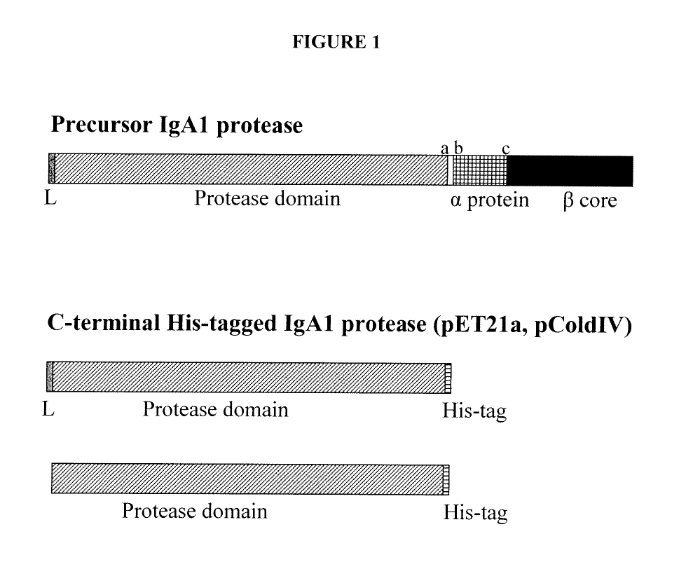

Serine-type IgA proteases differ in structure from the IgA metalloproteinase enzymes. Serine-type IgA proteases are initially produced as a precursor protein comprising a signal peptide, a protease domain, an α protein domain that is initially secreted with the protease domain, and a β-core domain that binds to the membrane of the cell, forming a pore to allow secretion of a protein comprising the α protein and the protease domain (FIG. 1). The β-core remains attached to the cell membrane while the protease domain and α domain are secreted. The α protein is later cleaved from the protease domain, resulting in the active mature IgA protease comprising only the proteolytic protease domain in the extracellular space (Vitovski et al., Infect. Immun., 75:2875-85 (2007)). H. influenzae, N. gonorrhoeae and N. meningitidis express IgA proteases of the serine endopeptidase family (Poulsen et al., J. Bacteriol., 174:2913-21 (1992)). Serine-type IgA proteases may be further grouped as type 1 or type 2 proteases based on the cleavage sites targeted in the IgA protein. For example, an H. influenzae type 1 IgA1 protease cleaves the human IgA1 hinge at the Pro-Ser bond between residues 231 and 232, whereas an H. influenzae type 2 IgA1 protease cleaves IgA1 at the Pro-Thr bond between residues 235 and 236. N. gnorrhoeae type 1 proteases cleave between Pro-Ser residues 237-238, whereas N. gnorrhoeae type 2 proteases cleave at the Pro-Thr bond between residues 235 and 236 (Lomholt et al., Mol. Microbiol., 15:495-506 (1995)). The target cleavage site is determined by the N-terminal portion of the proteolytic protease domain, which is comprised of approximately 1000 amino acids.

IgA-specific serine endopeptidases have been cloned from various bacteria, including without limitation N. gonorrhoeae, HF13, BK41, BK42, BK48, NG74, MC58, MS11, and F62; H. influenzae, Rd, HK61, HK224, HK284, HK368, HK393, HK635, HK715, HK869, and 86-028NP; and N. meningitidis, HF13, NGC80, NG117, NK183, NMB, FAM18, and MC58.

Exemplary IgA-specific serine endopeptidases produced according to the methods described herein, and used in the methods described herein, include without limitation those isolated from H. influenzae, N. gonorrhoeae and N. meningitidis, the amino acid sequences for some of which are disclosed in the following GenBank Accession Numbers, and IgA proteases having sequences substantially identical thereto, e.g., at least about 50%, 55%, 60%, 65%, 70%, 75%, 80%, 85%, 90%, 95%, 96%, 97%, 98% or 99% amino acid residue identity. In some embodiments, IgA-specific serine endopeptidases of at least about 60% or higher identity comprise only conservative substitutions. In other embodiments, IgA-specific serine endopeptidases of at least about 60% or higher identity comprise about 10% or less, or about 5% or less, non-conservative modifications (amino acid substitutions and/or additions).

Exemplary IgA proteases that can be produced according to the present methods, and can be used in the present methods, include without limitation: (H. influenzae) NP_{—}439153 (SEQ ID NO: 1), ABD78954.1 (SEQ ID NO: 2), YP_{—}248687 (SEQ ID NO: 3), AAX88027 (SEQ ID NO: 4), CAB56789 (SEQ ID NO: 5), ABG81065 (SEQ ID NO: 6), X59800 (SEQ ID NO: 23), which encodes the protein of SEQ ID NO: 5; (N. meningitidis) NP_{—}273742 (SEQ ID NO: 7), ABG81066 (SEQ ID NO: 8), AAC45792 (SEQ ID NO: 9); (N. gonorrhoeae) YP_{—}207437.1 (SEQ ID NO: 10), CAA00270 (SEQ ID NO: 11), CAA28538 (SEQ ID NO: 12). Additional IgA protease-producing bacterial strains and IgA protease sequence accession numbers are disclosed in Lomholt et al., Mol. Microbiol., 15:495-506 (1995), U.S. Pat. No. 7,407,653 and U.S. Patent Publication 2005/0136062, the disclosure of each of which is incorporated herein by reference in its entirety.

The present methods of producing IgA protease also encompass the use of polynucleotide sequences encoding the IgA protease polypeptides described herein, as well as other polynucleotide sequences that hybridize thereto under stringent or highly stringent conditions, and that encode polypeptides exhibiting serine-type IgA protease activity.

Production and Purification of IgA proteases

IgA protease polypeptides have been isolated from a number of bacterial strains that naturally produce IgA proteases, including serine-type IgA proteases from H. influenzae, N. gonorrhoeae and N. meningitidis. However, recovery of sufficiently large amount of IgA protease useful for administration to subjects suffering from an IgA deposition disease is not practicable from these strains. For example, isolation of naturally occurring or recombinant IgA protease from H. influenzae leads to only about 0.3 mg/L of IgA protease, and H. influenzae, which requires hemin for growth, is expensive to grow on a large scale for recombinant protein production.

Attempts have been made to clone IgA protease genes in alternative bacterial strains, such as E. coli. For example, a full-length serine-type IgA protease cloned from N. gonorrhoeae was expressed in E. coli and found to lack protease activity (Halter et al., EMBO J., 3:1595-1601 (1984), citing Koomey et al., Proc. Natl. Acad. Sci. U.S.A., 79:7881-85 (1982)). Halter et al. (supra) expressed a different IgA protease from N. gonorrhoeae, which resulted in some extracellular secretion of active enzyme. Khomenkov et al. (Mol. Genetics, Microbiol. and Virol., 22:34-40 (2007)) cloned two different N. meningitidis IgA proteases in E. coli. The IgA proteases were isolated as insoluble product from inclusion bodies. Grundy et al. (J. Bacteriol., 169:4442-50 (1987)) cloned H. influenzae IgA protease comprising the entire C-terminal portion of the protease protein into E. coli, resulting in a protease that was secreted into the culture media, in contrast to earlier studies with cloned H. influenzae IgA protease genes. Grundy et al. concluded that the C-terminal portion is important for protease secretion. U.S. Pat. No. 5,965,424 describes cloning of full-length IgA proteases in E. coli and reports that IgA protease is isolatable from inclusion bodies at approximately 6.2 g-10.4 g/10 L culture of cells, yielding 620-1040 mg of active, renatured IgA protease (per 10 L).

Vitovski et al., (Infect. Immun., 75:2875-85 (2007)) describe cloning of wild-type N. meningitidis IgA protease and IgA protease mutants having changes at the putative α and β domain cleavage sites (a, b and c in FIG. 1). Vitovski showed that when the putative cleavage sites were mutated, new sites for cleavage were used and mature protein was secreted into the cell media, albeit at slightly lower level compared to wild-type IgA protease. However, expression of a variant lacking the 32-amino acid peptide region between the a and b cleavage sites resulted in little to no protein secretion into the media, suggesting that the portion of the protein immediately C-terminal to the proteolytic protease domain is important for correct cleavage of the propeptide to form mature protein.

In some embodiments, the methods described herein do not utilize the full-length IgA protease gene as employed in previous attempts to produce IgA proteases in E. coli, but rather utilize a polynucleotide encoding an IgA protease polypeptide that comprises the proteolytic protease domain of a serine-type IgA protease and lacks at least some portion of the α protein domain and at least some portion of the β-core domain. In certain embodiments, the IgA protease polypeptides expressed or produced according to the present methods lack at least about 40%, 50%, 60%, 70%, 80%, 90%, 95% or 100% of the α protein domain, and at least about 40%, 50%, 60%, 70%, 80%, 90%, 95% or 100% of the β-core domain. In some embodiments, the polynucleotide encodes a polypeptide that comprises the proteolytic protease domain of a serine-type IgA protease and lacks 100% of the α protein domain and 100% of the β-core domain. Expression of a construct encoding a polypeptide that comprises the proteolytic protease domain of H. influenzae IgA1 protease and lacks 100% of the α protein domain and 100% of the β-core domain in E. coli cells resulted in substantially increased yield of soluble, active IgA1 protease present in the cell cytoplasm and/or periplasm, as well as substantially increased yield of soluble, active IgA1 protease formed from solubilization and refolding of inclusion bodies.

The methods described herein result in expression of an IgA protease (e.g., an IgA1 protease) as a soluble, active protein, and as inclusion bodies (insoluble, unfolded inactive protein aggregates) that can be isolated, washed/purified, solubilized and refolded into soluble, active IgA protease. Although inclusion bodies of a protein need to be solubilized and refolded to the native active form of the protein, expression of the protein as inclusion bodies may have advantages. For example, inclusion bodies may be expressed in higher yield, may be more protected from proteolytic degradation, and may be more pure prior to purification (e.g., using chromatography column(s)).

In additional embodiments, the IgA protease polypeptides expressed or produced according to the present methods comprise a signal sequence. In an embodiment, the signal sequence is an IgA protease signal sequence. In another embodiment, the signal sequence is a heterologous signal sequence. In further embodiments, the IgA protease polypeptides comprise amino acids derived from one or more heterologous polypeptides.

In some embodiments, the IgA protease polypeptides are recombinantly produced using techniques known in the art. See, e.g., Sambrook, Fritsch and Maniatis, Molecular Cloning: A Laboratory Manual, 2nd Ed., Cold Spring Harbor Laboratory Press (Cold Spring Harbor, N.Y. (1989)); DNA Cloning: A Practical Approach, Volumes I and II, D. N. Glover, Ed. (1985); and Current Protocols in Molecular Biology, John Wiley & Sons, Inc. (1994).

Recombinant polynucleotides encoding IgA protease polypeptides are expressed in an expression vector comprising a recombinant polynucleotide that contains expression control sequences operatively linked to a nucleotide sequence to be expressed. An expression vector comprises sufficient cis-acting elements for expression; other elements for expression can be supplied by the host cell or in vitro expression system. Expression vectors include all those known in the art, including without limitation cosmids, plasmids (e.g., naked or contained in liposomes) and viruses that incorporate the recombinant polynucleotide. The expression vector is inserted, via transformation or transfection, into an appropriate host cell for expression of the polynucleotide (see, e.g., Sambrook et al. (supra)).

Host cells useful for producing IgA proteases according to the present methods can be bacterial, yeast, plant, insect, non-mammalian vertebrate, or mammalian cells. Bacterial cells include gram-negative bacteria and gram-positive bacteria, e.g., any strain of E. coli, Bacillus, Streptomyces, Salmonella, and the like. Non-limiting examples of eukaryotic cells include insect cells (e.g., D. Mel-2, Sf4, Sf5, Sf9, Sf21, and High 5); plant cells; and yeast cells (e.g., Saccharomyces and Pichia). Mammalian cells include without limitation hamster, monkey, chimpanzee, dog, cat, bovine, porcine, mouse, rat, rabbit, sheep and human cells. The host cells can be immortalized cells (a cell line) or non-immortalized (primary or secondary) cells, and can be any of a wide variety of cell types, e.g., fibroblasts, keratinocytes, epithelial cells (e.g., mammary epithelial cells, intestinal epithelial cells), ovary cells (e.g., Chinese hamster ovary (CHO) cells), endothelial cells, glial cells, neural cells, formed elements of the blood (e.g., lymphocytes, bone marrow cells), chondrocytes and other bone-derived cells, and precursors of these somatic cell types. Mammalian host cells include without limitation CHO cells, baby hamster kidney (BHK) cells, human kidney 293 cells, COS-7 cells, HEK 293, SK-Hep, and HepG2. Host cells containing the polynucleotide encoding the IgA protease polypeptide are cultured under conditions appropriate for growth of the cells, expression of the polynucleotide, and identification/selection of cells expressing the IgA protease.

A wide variety of vectors can be used for the recombinant production of IgA protease polypeptides and can be selected from eukaryotic and prokaryotic expression vectors known in the art. Examples of vectors for prokaryotic expression include, but are not limited to, plasmids such as pRSET, pET, pBAD, pCold, pET21a, pColdIV, PHT01, pHT43 and others known in the art. Promoters useful in prokaryotic expression vectors include without limitation lac, trc, trp, recA, araBAD, T7, cold shock promoter, and others known in the art.

In some embodiments, a polynucleotide encoding an IgA protease polypeptide further encodes a signal peptide. In some embodiments, the signal peptide is derived from an IgA protease protein. In certain embodiments, the signal peptide is a serine-type IgA protease signal peptide. In other embodiments, the signal peptide is a heterologous signal peptide, and can be a signal peptide commonly used in the art for recombinant protein expression. As used herein, the term “heterologous signal peptide” refers to an amino acid or nucleotide sequence that is not naturally expressed in connection with the amino acid or nucleotide sequence to which it is operably linked. In the present disclosure, a heterologous signal peptide is not a serine-type IgA protease peptide. In further embodiments, the heterologous signal peptide is a cleavable peptide. Signal peptides useful for recombinant protein production are known to those of skill in the art.

In some embodiments, a polynucleotide sequence encoding an IgA protease polypeptide further encodes a cleavable or non-cleavable tag (e.g., a peptide tag, an epitope tag, etc.) useful for detection, isolation and/or purification of the polypeptide from the culture media or cell lysate. In certain embodiments, the cleavable or non-cleavable tag is a peptide tag, including without limitation a histidine (His) tag (e.g., a hexa-His tag), a peptide tag comprising a mixture of histidine, tyrosine and aspartate residues, a streptavidin-binding peptide sequence, a calmodulin-binding peptide sequence, or other peptide tag known in the art. In other embodiments, the tag is a FLAG or a c-Myc epitope tag useful in immunoprecipitation.

In further embodiments, the disclosure provides a host cell (e.g., a bacterial host cell) comprising a vector, the vector comprising a polynucleotide encoding a serine-type IgA protease polypeptide that comprises an IgA protease proteolytic domain and lacks an α protein domain and a β-core domain, wherein the IgA protease polypeptide is expressed from the host cell as insoluble inclusion bodies, or as a soluble polypeptide that exhibits IgA protease activity, or a combination thereof.

In additional embodiments, the disclosure provides a composition comprising at least about 50 grams or 75 grams wet weight of host cells expressing an IgA protease polypeptide as described herein. In certain embodiments, the wet weight of host cells is at least about 50, 55, 60, 65, 70, 75, 80, 85, 90, 95, 100, 125, 150, 200, 250, 300, 350, 400, 450, 500, 550, 600, 650, 700, 750, 800, 850, 900, 950 or 1000 grams or more of the host cells expressing an IgA protease polypeptide as described herein. It is further contemplated that the conditions described herein pertaining to the methods of the disclosure are also applicable to the host cell compositions described herein.

In some embodiments, the methods of the disclosure are carried out on a large scale and involve growing the host cells in a volume of at least about 10, 25, 50, 75 or 100 liters of culture medium. In certain embodiments, the methods involve growing host cells in a volume of at least about 10, 20, 30, 40, 50, 60, 70, 80, 90, 100, 125, 150, 175, 200, 250, 300, 350, 400, 450, 500, 600, 700, 800, 900, 1000, 1500, 2000, 2500, 3000, 3500, 4000, 4500, 5000, 5500, 6000, 6500, 7000, 7500, 8000, 9,000 or 10,000 or more liters of culture medium.

In other embodiments, the methods of the disclosure directly produce soluble, active IgA protease at a productivity level of at least about 10, 20, 30, 40, 50, 60, 70, 80, 90, 100, 110, 120, 130, 140, 150, 160, 170, 180, 190, 200, 210, 220, 230, 240, 250, 300, 350, 400, 450 or 500 mg/L or higher (mg of soluble, active IgA protease per liter of culture medium). Ranges encompassing any and all of these productivity level values are contemplated, e.g., about 20-40 mg/L, about 20-50 mg/L, about 20-70 mg/L, about 20-100 mg/L, and about 20-200 mg/L.

In yet other embodiments, the methods of the disclosure produce soluble, active IgA protease, by direct production and/or indirect production via inclusion bodies, at a productivity level of at least about 20, 40, 60, 80, 100, 150, 200, 250, 300, 350, 400, 450, 500, 550, 600, 650, 700, 750, 800, 850, 900, 950 or 1000 mg/L, or 2, 3, 4, 5, 6, 7, 8, 9, 10, 15 or 20 g/L (mg or grams of directly and/or indirectly produced soluble, active IgA protease per liter of culture medium).

In still other embodiments, the expression of an IgA protease polypeptide in host cells results in at least about 100% to about 1000%, including at least about 100%, 200%, 300%, 400%, 500%, 600%, 700%, 800%, 900% or 1000%, increased yield, or at least about 1000% to about 10,000%, including at least about 1000%, 2000%, 3000%, 4000%, 5000%, 6000%, 7000%, 8000%, 9000% or 10,000%, increased yield of soluble, active IgA protease, by direct production and/or indirect production via inclusion bodies, as compared to recombinant production of an IgA protease comprising the full-length serine-type IgA protease sequence.

The solubility of expressed IgA protease polypeptide can be increased in various ways. For example, the solubility of expressed protein can be affected by expression in different cell lines, including without limitation BL21, BL21(DE3), BL21Star™ (DE3), BL21(DE3)TrxB, BL21(DE3)pGro7/pG-KJE8/pKJE7/pG-Tf2/pTf16, ArcticExpress(DE3), C41(DE3), C43(DE3), Origami(DE3), Origami B(DE3), Tuner(DE3), KRX, and SHuffle™T7express, with or without pLysS. Further, the solubility of expressed protein can be increased by decreasing the rate of protein synthesis. The rate of protein synthesis can be modified by, e.g., variation of the temperature (e.g., about 10-40° C., about 10-30° C., about 20-30° C., about 0-30° C., about 0-20° C., about 0-15° C., or about 4-12° C.) at which the host cell is grown, use or non-use of an inducer and the choice of the inducer (e.g., IPTG), variation of the concentration of any inducer used, the choice and number of promoter(s), the number of plasmid copy(ies), and/or the nature of the culture medium. For example, the rate of protein synthesis can be decreased by growing the host cell at lower temperature (e.g., at a temperature from about 10° C. to about 28° C.) and/or lower concentration of inducer (e.g., about 0.4 mM IPTG) without significantly reducing cell growth rate. The solubility of expressed protein can also be increased by co-expression of chaperone(s) and/or foldase(s), including without limitation dnaK-dnaJ-grpE, groES-groEL, Cpn10-Cpn60, C1pB, and DsbC. Moreover, the solubility of expressed protein can be increased by use of an appropriate fusion partner, e.g., a carrier protein or fragment thereof, including without limitation glutathione-S-transferase (GST), maltose-binding protein (MBP), NusA, and SUMO. In addition, the solubility of expressed protein can be increased by its secretion into the periplasm using an appropriate leader sequence, such as pelB and ompT. The solubility of expressed protein can also be affected by the cell lysis conditions employed, including without limitation the use and choice of extraction buffer(s), detergent(s), and polymer(s) that prevent protein aggregation and help the protein remain soluble. Furthermore, solubility of expressed protein can be achieved by denaturing and refolding insoluble proteins (inclusion bodies) in vitro, and by employing, e.g., chaperone(s), foldase(s), high pressure, refolding buffer (e.g., Pierce refolding buffer, Hampton Research refolding buffer), and/or refolding kit (e.g., Novagen 96-well refolding kit, Takara refolding kit).

In additional embodiments, the amount of active protein produced or isolated is, e.g., at least about 10, 25, 50, 75, or 100 grams of active IgA protease, optionally combined with a pharmaceutically acceptable carrier, excipient or diluent or a sterile pharmaceutically acceptable carrier, excipient or diluent. In certain embodiments, the amount of active protein produced or isolated is at least about 10, 20, 30, 40, 50, 60, 70, 80, 90, 100, 125, 150, 175, 200, 300, 400, 500, 600, 700, 800, 900, 1000 or more grams of active IgA protease.

### Derivatives of IgA Protease Polypeptides

Polypeptide derivatives can be polypeptides chemically or non-chemically modified by such techniques as, for example and without limitation, ubiquitination, labeling (e.g., with radionuclides or various enzymes), covalent polymer attachment (e.g., derivatization with polyethylene glycol), and insertion or substitution by chemical or non-chemical synthesis of natural or unnatural amino acids (e.g., ornithine). Derivatives of an IgA protease are also useful as therapeutic agents and can be produced by the methods of the disclosure.

In some embodiments, one or more polyethylene glycol (PEG) groups are attached to the N-terminus, the C-terminus, and/or one or more internal sites of an IgA protease polypeptide produced by the methods of the disclosure. As used herein, the term “PEG” encompasses all the forms of PEG, linear and branched, which can be used to derivatize polypeptides, including without limitation mono-(C₁-C₁₀) alkoxy-PEGs and aryloxy-PEGs. PEGylation of an IgA protease can impart advantageous features to the protease, e.g., reduced immunogenicity, increased half-life, and/or reduced protein aggregation. The PEG groups can be of any convenient molecular weight, linear or branched, and monodispersed or polydispersed. In certain embodiments, the average molecular weight of a PEG group ranges from about 1 or 2 kiloDaltons (“kDa”) to about 100 kDa, or from about 1 or 2 kDa to about 50 kDa, or from about 5 kDa to about 50 kDa, or from about 2 kDa to about 20 kDa, or from about 5 kDa to about 20 kDa, or from about 2 kDa to about 10 kDa, or from about 5 kDa to about 10 kDa. The PEG groups can be attached to an IgA protease via, e.g., acylation or reductive alkylation involving a reactive group on the PEG moiety (e.g., an aldehyde, amino, thiol, ester, or activated ester group) and a reactive group on the protein moiety (e.g., an aldehyde, amino, or ester group) to form a hydrolysable or stable linkage (e.g., amide, imine, animal, alkylene, or ester bond). Addition of PEG moieties to polypeptides of interest can be carried out using techniques known in the art. See, e.g., International Publication No. WO 96/11953 and U.S. Pat. No. 4,179,337.

In some embodiments, one or more PEG groups are attached to the N-terminus, the C-terminus, and/or one or more internal sites of an IgA protease polypeptide, wherein each of the one or more PEG groups independently is linear or branched, is monodispersed or polydispersed, and has an average molecular weight from about 1 or 2 kDa to about 20 kDa, or from about 1 or 2 kDa to about 10 kDa. In certain embodiments, the one or more PEG groups independently have an average molecular weight of about 1, 2, 3, 4, 5, 6, 7, 8, 9, 10, 11, 12, 13, 14, 15, 16, 17, 18, 19 or 20 kDa.

Ligation of an IgA protease polypeptide with one or more PEG groups can take place in aqueous medium of appropriate pH (e.g., pH from about 5 to about 9, or pH from about 6 to about 9, or pH from about 7 to about 8.5), and can be monitored by reverse phase analytical HPLC. The PEGylated polypeptide can be purified by preparative HPLC and characterized by analytical HPLC, amino acid analysis and laser desorption mass spectrometry.

### IgA Deposition Disorders

The IgA proteases produced by the methods of the disclosure are useful for treating subjects suffering from IgA deposition disorders or diseases. An IgA deposition disorder or disease is characterized by formation of IgA antibody complexes in vivo and deposition of the IgA complexes in tissue(s) or other site(s) (e.g., organs) in the subject, resulting in adverse effects to the subject. IgA1 deposition is associated with a variety of clinical manifestations, such as renal failure, skin blistering, rash, arthritis, gastrointestinal bleeding and abdominal pain. Exemplary IgA deposition disorders include, but are not limited to, IgA nephropathy, hematuria, dermatitis herpetiformis, Henoch-Schoenlein purpura, Berger's disease, renal failure, liver disease due to IgA deposits, celiac disease, rheumatoid arthritis, Reiter's syndrome (reactive arthritis), ankylosing spondylitis, linear IgA disease, and HIV disorders (e.g., AIDS).

IgA nephropathy is characterized by IgA1 deposits within the kidney. The disease is an immune complex-mediated glomerulonephritis characterized by granular deposition of IgA1 in the glomerular mesangial areas of the kidney. The resulting nephropathy is associated with proliferative changes in the glomerular mesangial cells. Berger's disease is a form of IgA nephritis that can lead to renal failure. U.S. Pat. No. 7,407,653 and U.S. Patent Publication 2005/0136062 describe administration of IgA proteases from H. influenzae to treat IgA nephropathy, dermatitis herpetiformis, and Henoch-Schoenlein purpura. Lamm et al. (Am. J. Pathol., 172:31-36 (2008)) have studied the effects of administration of IgA proteases in a mouse model of IgA nephropathy. This study is discussed in Eitner et al., Nephrol. Dial. Transplant (2008).

Dermatitis herpetiformis is a chronic blistering disease characterized by deposits of IgA1 in skin and other tissues. Linear IgA disease is similar to dermatitis herpetiformis, and is a subepidermal blistering disease with histologic features indistinguishable from dermatitis herpetiformis. Linear IgA disease is characterized by a homogenous linear deposition of IgA along the dermo-epidermal junction in the skin (Leonard et al., J. R. Soc. Med., 75:224-237 (1982)).

Henoch-Schoenlein purpura affects the skin and kidneys. Henoch-Schoenlein purpura is characterized by deposition of IgA1-containing immune complexes in tissues and is diagnosed by observation of IgA1 deposition in the skin tissue or kidney (e.g., using immunofluorescence microscopy). Clinical manifestations typically include rash, arthralgia, abdominal pain, and renal disease.

Hematuria, the presence of red blood cells in urine, and proteinuria, the presence of protein in urine, are associated with IgA nephropathy, and can be indicative of early stage disease. Measurement of levels of hematuria and proteinuria is useful for assessing progression or improvement in IgA nephropathy in vivo.

Celiac disease is an inflammatory condition of the small intestine caused by the ingestion of wheat (or in some cases other gluten-containing products) in individuals having a certain genetic phenotype that confers sensitivity to gluten and wheat products. Gluten sensitivity can also manifest itself as a blistering, burning, itchy rash on the surface of the body (dermatitis herpetiformis). Celiac disease can result in circulating serum IgA complexes and deposition of the complexes in the kidneys (Pasternack et al., Clin. Nephrol., 34:56-60 (1990)).

Liver disease associated with IgA deposits (sometimes referred to as hepatic IgAN) is often observed in liver cirrhosis, chronic hepatitis and alcoholic liver disease. Symptoms include hematuria, proteinuria, elevated serum IgA levels and mesangial deposits of IgA. Severe cases can lead to end stage renal failure (Van De Wiel et al., Hepatology, 7:95-99 (2005)).

IgA deposition has been identified in a number of immunologic diseases affecting the joints (spondyloarthropathies), such as rheumatoid arthritis (Vetto et al., Rheumatol. Int., 10:13-19 (1990)), Reiter's disease and ankylosing spondylitis (Shu et al., Clin. Nephrol., 25:169-174 (1986)).

Animal models are available for studying IgA protease in treating IgA nephropathy (S. N. Emancipator et al., Animal models of IgA nephropathy, in IgA Nephropathy, pages 188-203, A. R. Clarkson, Editor, Martinus Nijhoff Publishing (Boston (1987)); U.S. Patent Publication 2005/0136062; Lamm et al., Am. J. Pathol., 172:31-36 (2008); and Gesualdo et al., J. Clin. Invest., 86:715-722 (1990)). In a model described by Gesualdo, an IgA antibody/dextran sulfate complex is injected into mice, resulting in deposits in the kidney and glomerulonephritis, which resembles human IgA nephropathy.

### Pharmaceutical Compositions of IgA Proteases and Methods of Using IgA Proteases

### Pharmaceutical Compositions of IgA Proteases

Further embodiments of the present disclosure relate to pharmaceutical compositions comprising an effective amount of an IgA protease (e.g., an IgA1 protease), and one or more pharmaceutically acceptable excipients, diluents, and/or carriers. The pharmaceutical compositions optionally comprise one or more other biologically active agents that may enhance the effects of the IgA protease and/or may exert other pharmacological effects in addition to those of the IgA protease. An effective amount of an active ingredient is a therapeutically, prophylactically or diagnostically effective amount, which can readily be determined by a person skilled in the art by taking into consideration such factors as the subject's body weight, age and condition, and therapeutic goal. In some embodiments, a condition or disorder associated with IgA deposition is treated or prevented by administering to a subject a pharmaceutical composition comprising an IgA protease.

In some embodiments, the compositions comprise active IgA protease in at least about 70%, 75%, 80%, 85%, 90%, 95%, 96%, 97%, 98% or 99% purity. In certain embodiments, the compositions contain less than about 10%, 5%, 4%, 3%, 2%, 1% or 0.5% of macromolecular contaminants, such as other mammalian (e.g., human) proteins and IgA protease aggregates.

Non-limiting examples of excipients, carriers and diluents include vehicles, liquids, buffers, isotonicity agents, additives, stabilizers, preservatives, solubilizers, surfactants, emulsifiers, wetting agents, adjuvants, and so on. The compositions can contain liquids (e.g., water, ethanol); diluents of various buffer content (e.g., Tris-HCl, phosphate, acetate buffers, citrate buffers), pH and ionic strength; detergents, surfactants and solubilizing agents; anti-adsorbents (e.g., Polysorbate 20, Polysorbate 80, benzyl alcohol); anti-oxidants (e.g., methionine, ascorbic acid, sodium metabisulfite); preservatives (e.g., Thimerosol, benzyl alcohol, m-cresol); bulking substances (e.g., lactose, mannitol, sucrose); or combinations thereof. The use of excipients, diluents and carriers in the formulation of pharmaceutical compositions is known in the art; see, e.g., Remington's Pharmaceutical Sciences, 18th Edition, pages 1435-1712, Mack Publishing Co. (Easton, Pa. (1990)), which is incorporated herein by reference in its entirety.

For example, carriers include without limitation diluents, vehicles and adjuvants, as well as implant carriers, and inert, non-toxic solid or liquid fillers and encapsulating materials that do not react with the active ingredient(s). Non-limiting examples of carriers include phosphate buffered saline, physiological saline, water, and emulsions (e.g., oil/water emulsions). A carrier can be a solvent or dispersing medium containing, e.g., saline, an alcohol (e.g., ethanol), a polyol (e.g., glycerol, propylene glycol, liquid polyethylene glycol, and the like), a vegetable oil (e.g., olive oil), an organic ester (e.g., ethyl oleate), a cyclodextrin (e.g., beta-cyclodextrin) or modified cyclodextrin (e.g., a sulfobutyl ether cyclodextrin), hyaluronic acid, and mixtures thereof.

In further embodiments, pharmaceutical compositions comprising an IgA protease contain a buffer solution or a buffering agent to maintain the pH of a solution or suspension within a desired range. Non-limiting examples of buffer solutions include phosphate buffered saline, Tris buffered saline, and Hank's buffered saline. Exemplary buffering agents include sodium acetate, sodium phosphate, and sodium citrate. Mixtures of buffering agents can also be used. In certain embodiments, the buffering agent is acetic acid/acetate or citric acid/citrate. The amount of buffering agent suitable in the composition depends in part on the particular buffer used and the desired pH of the solution or suspension. For example, acetate is a more efficient buffer at pH 5 than pH 6, so less acetate may be used in a solution at pH 5 than at pH 6. In certain embodiments, the pH range for the compositions of the present disclosure is from about 3 to about 7.5, or from about 4 to about 7, or from about 5 to about 7, or from about 6 to about 7, or from about 4 to about 6, or from about 4 to about 5, or from about 5 to about 6.

In other embodiments, the compositions contain an isotonicity-adjusting agent to render the solution or suspension isotonic and more compatible for injection. Non-limiting examples of isotonicity agents include NaCl, dextrose, glucose, glycerin, sorbitol, xylitol, and ethanol. In certain embodiments, the isotonicity agent is NaCl. In certain embodiments, NaCl is at a concentration of about 160±20 mM, or about 140 mM±20 mM, or about 120±20 mM, or about 100 mM±20 mM, or about 80 mM±20 mM, or about 60 mM±20 mM.

In yet other embodiments, the compositions comprise one or more preservatives. Preservatives include, but are not limited to, m-cresol and benzyl alcohol. Preservatives can also be antibacterial agents and antifungal agents that suppress the action of microorganisms, such as paraben, chlorobutanol, phenol sorbic acid and the like. In certain embodiments, the one or more preservatives independently are at a concentration of about 0.1%, or about 0.4%±0.2%, or about 1%±0.5%, or about 1.5%±0.5%, or about 2.0%±0.5%.

In still other embodiments, the compositions comprise a stabilizer. Non-limiting examples of stabilizers include glycerin, glycerol, thioglycerol, methionine, and ascorbic acid and salts thereof.

Pharmaceutically acceptable salts can be used in the compositions, including without limitation mineral acid salts (e.g., hydrochloride, hydrobromide, phosphate, sulfate), salts of organic acids (e.g., acetate, propionate, malonate, benzoate, mesylate, tosylate), and salts of amines (e.g., isopropylamine, trimethylamine, dicyclohexylamine, diethanolamine). A thorough discussion of pharmaceutically acceptable salts is found in Remington's Pharmaceutical Sciences, 18th Edition, Mack Publishing Company, (Easton, Pa. (1990)).

The pharmaceutical compositions can be administered in various forms, such as tablets, capsules, granules, powders, solutions, suspensions, emulsions, ointments, and transdermal patches. The dosage forms of the compositions can be tailored to the desired mode of administration of the compositions. For oral administration, the compositions can take the form of, e.g., a tablet or capsule (including softgel capsule), or can be, e.g., an aqueous or nonaqueous solution, suspension or syrup. Solid dosage forms (e.g., tablets, capsules) for oral administration can include one or more commonly used excipieints, diluents and carriers, such as mannitol, lactose, glucose, sucrose, starch, corn starch, sodium saccharin, talc, cellulose, magnesium carbonate, and lubricating agents (e.g., magnesium stearate, sodium stearyl fumarate). If desired, flavoring, coloring and/or sweetening agents can be added to solid and liquid formulations. Other optional ingredients for oral formulations include without limitation preservatives, suspending agents, and thickening agents.

Formulations for parenteral administration can be prepared, e.g., as liquid solutions or suspensions, as solid forms suitable for solubilization or suspension in a liquid medium prior to injection, or as emulsions. For example, sterile injectable solutions and suspensions can be formulated according to techniques known in the art using suitable diluents, carriers, vehicles (e.g., isotonic vehicles, such as Sodium Chloride Injection, Ringer's Injection, Dextrose Injection, Dextrose and Sodium Chloride Injection, Lactated Ringer's Injection), solvents (e.g., buffered aqueous solution, Ringer's solution, isotonic sodium chloride solution), dispersing agents, wetting agents, emulsifying agents, suspending agents, and the like. In addition, sterile fixed oils, fatty esters, polyols and/or other inactive ingredients can be used. As further examples, formulations for parenteral administration include aqueous sterile injectable solutions, which can contain antioxidants, buffers, bacteriostats, and solutes that render the formulation isotonic with the blood of the intended recipient; and aqueous and nonaqueous sterile suspensions, which can contain suspending agents and thickening agents. Prolonged absorption of an injectable formulation can be achieved by the inclusion of agents that delay absorption, such as aluminum monostearate and gelatin.

Liquid formulations can be prepared, e.g., by dissolving, mixing, dispersing or suspending an active agent and one or more excipients, diluents and/or carriers in a liquid medium containing, e.g., water, saline, aqueous dextrose, glycerol, ethanol, or a combination thereof, to form a solution or suspension. If desired, the formulations can contain a variety of excipeints, such as dispersing agents, wetting agents, emulsifying agents, suspending agents, pH buffering agents, isotonicity agents, sodium acetate, sorbitan monolaurate, triethanolamine sodium acetate, triethanolamine oleate, etc. Methods of preparing solid and liquid dosage forms are known, or will be apparent, to those skilled in the art (see, e.g., Remington's Pharmaceutical Sciences, (supra)).

Compositions comprising an IgA protease can also be lyophilized formulations. In certain embodiments, the lyophilized formulations comprise a buffer and bulking agent, and optionally a stabilizer or antioxidant. Exemplary buffers include without limitation acetate buffers and citrate buffers. Exemplary bulking agents include without limitation mannitol, sucrose, dexran, lactose, trehalose, and povidone (PVP K24).

The disclosure also provides kits containing, e.g., vials, ampoules, tubes or bottles that comprise a sterile injectable formulation or lyophilized formulation. Furthermore, extemporaneous injection solutions and suspensions can be prepared from, e.g., sterile powder, granules or tablets comprising the IgA protease-containing composition. The kits can also include dispensing devices, such as an aerosol or injection dispensing device, syringe and/or needle, and instructions for use.

In addition, pharmaceutical compositions comprising an IgA protease can be formulated as a slow release, controlled release or sustained release system for maintaining a relatively constant level of dosage over a desired time period, e.g., 1 week, 2 weeks, 3 weeks, 1 month, 2 months or 3 months. Slow release, controlled release and sustained release formulations can be prepared using, e.g., biodegradable polymeric systems (which can comprise, e.g., hydrophilic polymers), and can take the form of, e.g., microparticles, microspheres or liposomes, as is known in the art.

Slow release, controlled release and sustained release formulations can be, e.g., a matrix made of materials (e.g., polymers) that are degradable by enzymatic or acid/base hydrolysis or by dissolution. Once introduced into the body, the matrix is acted upon by enzymes and body fluids. The matrix is made of biocompatible materials such as polylactide, polyglycolide, poly(lactide-co-glycolide), polyanhydrides, polyorthoesters, polyproteins, hyaluronic acid, collagen, chondroitin sulfate, carboxylic acids, fatty acids, phospholipids, polysaccharides, nucleic acids, polyamino acids, amino acids (e.g., phenylalanine, tyrosine, isoleucine), polynucleotides, polyvinyl propylene, polyvinylpyrrolidone, and silicone.

### Dosages

As used herein, the term “therapeutically effective amount” of an active agent (e.g., an IgA protease) refers to an amount that provides therapeutic benefit to a patient. The amount may vary from one individual to another and may depend upon a number of factors, including the overall physical condition of the patient. A therapeutically effective amount of an IgA protease can be readily ascertained by one skilled in the art, using publicly available materials and procedures. In one embodiment, the subject to be treated is a mammal. In a related embodiment, the subject is a human.

The dosing frequency for a particular subject may vary depending upon various factors, including the disorder being treated and the condition and response of the subject to the therapy. In some embodiments, a pharmaceutical composition comprising an IgA protease (e.g., an IgA1 protease) is administered to a subject one time per day, per two days, per three days, per week, per two weeks, per month, per two months, or per three months. In certain embodiments, a daily or weekly dose of an IgA protease is administered to a subject to treat or prevent an IgA deposition disorder (e.g., IgA nephropathy, hematuria, dermatitis herpetiformis, Henoch-Schoenlein purpura, Berger's disease, renal failure, liver disease, celiac disease, rheumatoid arthritis, Reiter's disease, ankylosing spondylitis, linear IgA disease, or HIV disorders such as AIDS).

An IgA protease (e.g., an IgA1 protease) is administered to a subject at a therapeutically effective dose to treat or prevent an IgA deposition disorder (e.g., IgA nephropathy, hematuria, dermatitis herpetiformis, Henoch-Schoenlein purpura, Berger's disease, renal failure, liver disease, celiac disease, rheumatoid arthritis, Reiter's disease, ankylosing spondylitis, linear IgA disease, or HIV disorders such as AIDS). The safety and efficacy of an IgA protease can be evaluated using standard pharmacological procedures in cell cultures and experimental animals (e.g., rodents, primates), e.g., by determining the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD₅₀/ED₅₀. Active agents exhibiting a large therapeutic index are normally preferred.

Data obtained from cell culture assays and animal studies can be used to calculate a range of dosage for use in humans. The dosage normally lies within a range of circulating concentrations that include the ED₅₀, with minimal or no toxicity. The dosage can vary within this range depending upon, e.g., the dosage form and route of administration utilized.

In certain embodiments, a single dose of an IgA protease is from about 0.1 mg/kg to about 10 mg/kg body weight, or from about 0.5 mg/kg to about 5 mg/kg. The dosage and frequency of administration of an IgA protease can be adjusted according to, e.g., the degree of affliction and the subject's response to the therapy.

### Modes of Administration

An IgA protease (e.g., an IgA1 protease), or a pharmaceutical composition comprising an IgA protease, can be administered to a subject in various ways. In general, an IgA protease can be administered as a pharmaceutical formulation suitable for oral (including buccal and sub-lingual), rectal, nasal, topical, pulmonary, vaginal or parenteral (including intramuscular, intraarterial, intrathecal, subcutaneous and intravenous) administration, or in a form suitable for administration by inhalation or insufflation.

In some embodiments, an IgA protease is administered by injection or infusion subcutaneously, intravenously, intra-arterially, intraperitoneally, intramuscularly, intrasternally, intradermally or intrathecally. In certain embodiments, an IgA protease is administered to a subject by a single subcutaneous, intravenous, intra-arterial, intraperitoneal, intramuscular, intrasternal, intradermal or intrathecal injection once a day, once a week, twice a week, once every two weeks, or once a month. An IgA protease can also be administered by direct injection at or near the site(s) of IgA deposition.

Furthermore, an IgA protease can be administered by injection or implantation of a depot at or near the target site(s) of action (e.g., kidney, liver, skin). Injectable depot formulations can made by forming microencapsule matrices of the therapeutic agent in biodegradable polymers (e.g., polylactide, polyglycolide, polyorthoesters, polyanhydrides, and copolymers thereof). Depending upon the ratio of therapeutic agent to polymer and the nature of the particular polymer employed, the rate of therapeutic agent release can be controlled. Injectable depot formulations can also be prepared by entrapping the therapeutic agent in liposomes or microemulsions that are compatible with body tissues.

Alternatively, an IgA protease can be administered under the tongue (e.g., sublingual tablet) or by inhalation into the lungs (e.g., inhaler or aerosol spray), by delivery into the nasal cavity (e.g., intranasal spray), by delivery into the eye (e.g., eye drop), or by transdermal delivery (e.g., by means of a patch on the skin). An IgA protease can also be administered orally in the form of microspheres, microcapsules, liposomes (uncharged or charged (e.g., cationic)), polymeric microparticles (e.g., polyamides), microemulsions, and the like. It will be apparent to one skilled in the art that an IgA protease can also be administered by other modes and methods, and determination of the most effective mode and method of administration of the IgA protease is within the skill of the skilled artisan.

Another method of administration of an IgA protease is by osmotic pump (e.g., an Alzet pump) or mini-pump, which allows for controlled, continuous delivery of the IgA protease over a pre-determined period. The osmotic pump or mini-pump can be implanted subcutaneously, or near the target site (e.g., the kidney, liver or skin).

For local delivery of an IgA protease to the diseased area (e.g., tissue), the IgA protease can be delivered by means of a medical device implanted at the diseased site. In one embodiment, the IgA protease is impregnated in a polymeric matrix or polymeric coating disposed over the device. In another embodiment, the IgA protease is contained in reservoirs or channels formed in the body of the device and covered by a porous polymeric membrane or layer through which the IgA protease can diffuse. The polymeric matrix, coating, membrane or layer can comprise at least one biodegradable (e.g., hydrophilic) polymer, as is known in the art. In a further embodiment, the IgA protease can be contained in micropores in the body of the device. The IgA protease can be delivered from the device by burst release, pulse release, controlled release or sustained release, or a combination thereof. For example, the medical device can locally deliver the IgA protease to the diseased site in a burst release followed by a sustained release. Sustained release can be over a period up to about 1 week, 2 weeks, 1 month, 2 months, 3 months, 6 months or 1 year.

Depending on the intended mode of administration, a pharmaceutical composition comprising an IgA protease can be in the form of solid, semi-solid or liquid dosage forms, such as tablets, suppositories, pills, capsules, powders, liquids, suspensions, creams, ointments, lotions, and the like, preferably in unit dosage form suitable for single administration of a precise dosage. The composition contains an effective amount of the IgA protease (e.g., an IgA1 protease) and one or more pharmaceutically acceptable excipients, carriers and/or diluents, and optionally one or more other bioactive agents.

### Combination Therapy

In some embodiments, an IgA protease (e.g., an IgA1 protease), or a pharmaceutical composition comprising an IgA protease, is used in combination with one or more other active agents useful for treating or preventing conditions and disorders associated with IgA deposition, such as liver and kidney disorders (e.g., IgA nephropathy). The other active agent(s) can enhance the effects of the IgA protease and/or exert other pharmacological effects in addition to those of the IgA protease. Non-limiting examples of active agents that can be used in combination with an IgA protease described herein are immunosuppressants (e.g., cyclosporine, azathioprine), corticosteroids, anti-inflammatory agents, dietary fish oil supplements (e.g., to reduce renal inflammation), and angiotensin-converting enzyme inhibitors (e.g., to reduce the risk of progressive renal disease and renal failure).

To achieve a desired therapeutic outcome in a combination therapy, an IgA protease and other active agent(s) are generally administered to a subject in a combined amount effective to produce the desired therapeutic outcome (e.g., reduction or elimination of IgA deposition in tissues or inflammation associated with such deposition). The combination therapy can involve administering the IgA protease and the other active agent(s) at about the same time. Simultaneous administration can be achieved by administering a single composition that contains both the IgA protease and the other active agent(s). Alternatively, the other active agent(s) can be taken separately at about the same time as a pharmaceutical formulation (e.g., solid or semi-solid dosage form, injection or drink) comprising the IgA protease.

In other alternatives, administration of the IgA protease can precede or follow administration of the other active agent(s) by an interval ranging from minutes to hours. In embodiments where the IgA protease and the other active agent(s) are administered at different times, the IgA protease and the other active agent(s) are administered within an appropriate time of one another so that both the IgA protease and the other active agent(s) can exert a beneficial effect (e.g., synergistically or additively) on the patient. In some embodiments, the IgA protease is administered to the subject within about 0.5-12 hours (before or after), or within about 0.5-6 hours (before or after), of the other active agent(s). In certain embodiments, the IgA protease is administered to the subject within about 0.5 hour or 1 hour (before or after) of the other active agent(s).

### Kits

The disclosure also provides kits containing an IgA protease (e.g., an IgA1 protease) described herein, or a pharmaceutical composition comprising an IgA protease. The kit can also contain one or more other active agents useful for treating or preventing an IgA deposition disorder. In some embodiments, an IgA protease (and optionally other active agent(s)) is contained in a storage container or vessel, such as a vial, ampoule, bottle, bag, reservoir, tube, blister, pouch, patch and the like. The IgA protease (and optionally other active agent(s)) can be provided in liquid form (e.g., a sterile injectable solution), or in semi-solid or solid form (e.g., frozen, lyophilized, freeze-dried, spray freeze-dried, or any other reconstitutable form) that can be reconstituted to a desired form (e.g., injectable solution or suspension). Any of various reconstitution media can be provided in the kit.

The kit can also contain suitable device(s) (e.g., syringe, needle, other injection device, etc.) for administering the IgA protease (and optionally other active agent(s)) to the subject. Furthermore, the kit can include instructions for preparing and administering the IgA protease (and optionally other active agent(s)).

### Representative Embodiments of the Disclosure

Certain embodiments of the disclosure relate to a method for producing a serine-type IgA protease in/from a host cell, comprising growing a host cell comprising a vector, the vector comprising a polynucleotide encoding an IgA protease polypeptide that comprises an IgA protease proteolytic domain and lacks an α protein domain and a β-core domain, under conditions that result in expression of the IgA protease polypeptide as inclusion bodies, or as a soluble polypeptide that exhibits IgA protease activity, or a combination thereof.

In some embodiments, the method further comprises transforming the host cell with the vector prior to growing the host cell.

In some embodiments, the IgA protease polypeptide expressed or produced according to the method lacks at least about 40%, 50%, 60%, 70%, 80%, 90%, 95% or 100% of the α protein domain, and lacks at least about 40%, 50%, 60%, 70%, 80%, 90%, 95% or 100% of the β-core domain, or any combination of the aforementioned percentages. In certain embodiments, the IgA protease polypeptide expressed or produced according to the method lacks at least about 50% of the α protein domain and at least about 50% of the β-core domain. In an embodiment, the IgA protease polypeptide expressed or produced according to the method lacks 100% of the α protein domain and 100% of the β-core domain.

In certain embodiments, the method further comprises isolating the inclusion bodies, solubilizing the isolated inclusion bodies, and refolding the solubilized inclusion bodies to/into soluble, active IgA protease.

In some embodiments, the isolated inclusion bodies are solubilized using a chaotropic agent selected from the group consisting of urea, guanidine hydrochloride (guanidinium chloride), lithium perchlorate, formic acid, acetic acid, trichloroacetic acid, sulfosalicylic acid, sarkosyl (sodium lauroyl sarcosinate), and combinations thereof. In certain embodiments, the chaotropic agent is urea or guanidine hydrochloride. In some embodiments, the chaotropic agent is at a concentration from about 4 M to about 10 M. In certain embodiments, the chaotropic agent is at about 4, 4.5, 5, 5.5, 6, 6.5, 7, 7.5, 8, 8.5, 9, 9.5 or 10 M. In certain embodiments, the chaotropic agent is about 6 M guanidine hydrochloride or about 8 M urea.

In further embodiments, the solubilized inclusion bodies are refolded in a refolding buffer that comprises Tris [tris(hydroxymethyl)aminomethane] and NaCl, and has a pH from about 7 to about 9.5. In some embodiments, the pH of the Tris refolding buffer is from about 7.5 to about 9. In certain embodiments, the pH of the Tris refolding buffer is about 7.5, 7.6, 7.7, 7.8, 7.9, 8, 8.1, 8.2, 8.3, 8.4, 8.5, 8.6, 8.7, 8.8, 8.9 or 9.

In other embodiments, the solubilized inclusion bodies are refolded in a refolding buffer that comprises CHES (N-cyclohexyl-2-aminoethanesulfonic acid) and NaCl, and has a pH from about 8 to about 10. In some embodiments, the pH of the CHES refolding buffer is from about 8.5 to about 10, or from about 8.5 to about 9.5. In certain embodiments, the pH of the CHES refolding buffer is about 8.5, 8.6, 8.7, 8.8, 8.9, 9, 9.1, 9.2, 9.3, 9.4, 9.5, 9.6, 9.7, 9.8, 9.9 or 10.

In still other embodiments, the solubilized inclusion bodies are refolded in a refolding buffer that comprises MES [2-(N-morpholino)ethanesulfonic acid] and NaCl, and has a pH from about 5 to about 7. In some embodiments, the pH of the MES refolding buffer is from about 5.5 to about 7, or from about 5.5 to about 6.5. In certain embodiments, the pH of the MES refolding buffer is about 5.5, 5.6, 5.7, 5.8, 5.9, 6, 6.1, 6.2, 6.3, 6.4, 6.5, 6.6, 6.7, 6.8, 6.9 or 7.

In yet other embodiments, the solubilized inclusion bodies are refolded in a refolding buffer that comprises phosphate-buffered saline (PBS), and has a pH from about 6 to about 8. In some embodiments, the pH of the PBS refolding buffer is from about 6.5 to about 8, or from about 7 to about 8. In certain embodiments, the pH of the PBS refolding buffer is about 6.5, 6.6, 6.7, 6.8, 6.9, 7, 7.1, 7.2, 7.3, 7.4, 7.5, 7.6, 7.7, 7.8, 7.9 or 8.

In additional embodiments, the refolding buffer comprising Tris, CHES, MES or PBS further comprises arginine. In some embodiments, the concentration of arginine in the refolding buffer is about 0.05 M to about 1.5M. In certain embodiments, the concentration of arginine in the refolding buffer is about 0.1, 0.2, 0.3, 0.4, 0.5, 0.6, 0.7, 0.8, 0.9 or 1 M. In other embodiments, the refolding buffer comprising Tris, CHES, MES or PBS, and optionally arginine, further comprises guanidine hydrochloride or urea.

In some embodiments, the solubilized inclusion bodies are refolded at a temperature from about 4° C. to about 30° C. In certain embodiments, the solubilized inclusion bodies are refolded at about 4, 10, 15, 20, 22, 25 or 30° C. In certain embodiments, the solubilized inclusion bodies are refolded at about 4° C. or ambient temperature.

In further embodiments, the solubilized inclusion bodies are at a concentration from about 0.01 mg/mL to about 1 mg/mL, or from about 0.01 mg/mL to about 2 mg/mL, in the refolding solution or mixture. In some embodiments, the solubilized inclusion bodies are at a concentration of about 0.025, 0.05, 0.1, 0.2, 0.3, 0.4 or 0.5 mg/mL in the refolding solution or mixture. In certain embodiments, the solubilized inclusion bodies are at a concentration of about 0.05, 0.1 or 0.2 mg/mL in the refolding solution or mixture.

In other embodiments, the isolated inclusion bodies are solubilized using urea, and the solubilized inclusion bodies are refolded in a refolding buffer that comprises Tris, lacks added arginine, and has a pH from about 7.5 to about 9.5. In some embodiments, the isolated inclusion bodies are solubilized using urea at a concentration from about 6 M to about 10 M. In certain embodiments, the isolated inclusion bodies are solubilized using urea at about 6, 6.5, 7, 7.5, 8, 8.5, 9, 9.5 or 10 M. In some embodiments, the pH of the refolding buffer is from about 7.7 to about 9. In certain embodiments, the pH of the refolding buffer is about 7.7, 7.8, 7.9, 8, 8.1, 8.2, 8.3, 8.4, 8.5, 8.6, 8.7, 8.8, 8.9 or 9. In some embodiments, the concentration of Tris in the refolding buffer is from about 20 mM to about 100 mM. In certain embodiments, the concentration of Tris in the refolding buffer is about 20, 25, 30, 35, 40, 45, 50, 55, 60, 65, 70, 75, 80, 85, 90, 95 or 100 mM.

In additional embodiments, the refolding buffer lacking added arginine further comprises NaCl or glycerol, or a combination thereof. In some embodiments, the refolding buffer comprises NaCl at a concentration from about 10 mM to about 500 mM, or glycerol at a concentration from about 1% to about 20%, or a combination thereof. In certain embodiments, the refolding buffer comprises NaCl at about 10, 20, 30, 40, 50, 60, 70, 80, 90, 100, 150, 200, 250, 300, 350, 400, 450 or 500 mM, or glycerol at about 2%, 4%, 6,%, 8%, 10%, 12%, 14%, 16%, 18% or 20%, or a combination thereof.

In further embodiments, the isolated inclusion bodies are solubilized using about 7-9 M urea, and the solubilized inclusion bodies are refolded in a refolding buffer that lacks added arginine, has a pH from about 7.8 to about 9, and comprises (a) about 30-70 mM Tris, or (b) about 30-70 mM Tris and about 50-250 mM NaCl, or (c) about 30-70 mM Tris and about 5-15% glycerol. In certain embodiments, the isolated inclusion bodies are solubilized using about 8 M urea, and the solubilized inclusion bodies are refolded in a refolding buffer that lacks added arginine, has a pH from about 8 to about 9, and comprises (a) about 50 mM Tris, or (b) about 50 mM Tris and about 100 mM (0.1 M) NaCl, or (c) about 50 mM Tris and about 10% glycerol.

In some embodiments, the solubilized inclusion bodies are refolded in the absence of added arginine at a temperature from about 4° C. to about 30° C. In certain embodiments, the solubilized inclusion bodies are refolded at about 4, 10, 15, 20, 22, 25 or 30° C. In an embodiment, the solubilized inclusion bodies are refolded at ambient temperature.

In additional embodiments, the method further comprises washing/purifying the isolated inclusion bodies prior to solubilizing the isolated inclusion bodies. In some embodiments, the washing/purifying comprises using a surfactant or detergent. In certain embodiments, the surfactant or detergent is an alkyl poly(ethylene oxide) or alkylphenol poly(ethylene oxide) surfactant or detergent. In an embodiment, the surfactant or detergent is Triton X-100. In further embodiments, the washing/purifying comprises centrifuging the isolated inclusion bodies or microfiltering the isolated inclusion bodies through a hollow fiber with crossflow filtration.

In other embodiments, the method further comprises purifying the refolded IgA protease. In some embodiments, the purifying comprises ultrafiltrating and diafiltrating (UF/DF) the refolded IgA protease. In certain embodiments, the purifying comprises using a nickel column (e.g., a Nickel IMAC Chelating Sepharose column (GE Healthcare, Piscataway, N.J.)), an anion-exchange column (e.g., a Q sepharose column (GE Healthcare), a GigaCap Q column (Tosoh BioSciences, South San Francisco, Calif.)), a cation-exchange column, a hydrophobic-interaction column (e.g., a butyl sepharose 4 column (GE Healthcare), a reverse-phase HPLC column), or a size-exclusion column (e.g., an S300 Sephacryl column (GE Healthcare)), or a combination thereof.

In certain embodiments, the method results in at least about 1, 2, 3, 4, 5, 6, 7, 8, 9, 10, 15 or 20 g/L of soluble, active IgA protease from at least about 10, 20, 30, 40, 50, 60, 70, 80, 90, 100, 150 or 200 g/L of IgA protease inclusion bodies. In some embodiments, the method results in at least about 1-2 g/L of soluble, active IgA protease from at least about 10-20 g/L of IgA protease inclusion bodies.

In yet other embodiments, the method further comprises isolating the soluble polypeptide that exhibits IgA protease activity (soluble, active IgA protease polypeptide). In additional embodiments, the method further comprises purifying the isolated IgA protease polypeptide. In certain embodiments, the purifying comprises using a nickel column (e.g., a Nickel IMAC Chelating Sepharose column), an anion-exchange column (e.g., a Q sepharose column, a GigaCap Q column), a cation-exchange column, a hydrophobic-interaction column (e.g., a butyl sepharose 4 column, a reverse-phase HPLC column), or a size-exclusion column (e.g., an S300 Sephacryl column), or a combination thereof.

In some embodiments, the method results in at least about 10, 20, 30, 40, 50, 60, 70, 80, 90, 100, 150, 200, 250, 300, 350, 400, 450 or 500 mg/L of soluble, active IgA protease polypeptide. In certain embodiments, the method results in at least about 20-40 mg/L of soluble, active IgA protease polypeptide.

In other embodiments, the expression of IgA protease polypeptide results in a ratio of mg soluble, active IgA protease polypeptide produced to mg total IgA protease polypeptide produced of at least about 0.5%, 1%, 2%, 3%, 4%, 5%, 6%, 7%, 8%, 9% or 10%. In certain embodiments, the expression of IgA protease polypeptide results in a ratio of mg soluble, active IgA protease polypeptide produced to mg total IgA protease polypeptide produced of at least about 0.5% or 1%.

In still other embodiments, the growing of the host cell comprising the vector results in at least about a 10-, 20-, 30-, 40-, 50-, 60-, 70-, 80-, 90-, 100-, 200-, 300-, 400-, 500-, 600-, 700-, 800-, 900- or 1000-fold higher production of soluble, active IgA protease, by direct production or indirect production via inclusion bodies, or both, compared to culturing under the same conditions a host cell comprising a vector that encodes the entirety of the α protein domain and the β-core domain. In certain embodiments, the growing of the host cell comprising the vector results in at least about a 10-, 50-, 100-, 500- or 1000-fold higher production of soluble, active IgA protease, by direct production or indirect production via inclusion bodies, or both, compared to culturing under the same conditions a host cell comprising a vector that encodes the entirety of the α protein domain and the β-core domain.

In certain embodiments, the IgA protease polypeptide expressed in/from the host cell comprises a histidine tag (e.g., a hexa-histidine tag). Inclusion bodies of an IgA protease comprising a histidine tag can be solubilized and refolded as described herein. Furthermore, soluble, active IgA protease comprising a histidine tag, and refolded IgA protease comprising a histidine tag, can be purified using any of the methods and techniques described herein. As a non-limiting example, an IgA protease comprising a histidine tag can be purified using a nickel column (e.g., a Nickel IMAC Chelating Sepharose column), an anion-exchange column (e.g., a Q sepharose column, a GigaCap Q column), a cation-exchange column, a hydrophobic-interaction column (e.g., a butyl sepharose 4 column, a reverse-phase HPLC column), or a size-exclusion column (e.g., an S300 Sephacryl column), or a combination thereof (e.g., a nickel column, followed by an anion-exchange column or a hydrophobic-interaction column, followed by a size-exclusion column).

In other embodiments, the IgA protease polypeptide expressed in/from the host cell does not comprise a histidine tag. Inclusion bodies of an IgA protease lacking a histidine tag can be solubilized and refolded as described herein. Moreover, soluble, active IgA protease lacking a histidine tag, and refolded IgA protease lacking a histidine tag, can be purified using any of the methods and techniques described herein. As a non-limiting example, an IgA protease lacking a histidine tag can be purified using an anion-exchange column (e.g., a Q sepharose column, a GigaCap Q column), a cation-exchange column, a hydrophobic-interaction column (e.g., a butyl sepharose 4 column, a reverse-phase HPLC column), or a size-exclusion column (e.g., an S300 Sephacryl column), or a combination thereof (e.g., an anion-exchange column, followed by a hydrophobic-interaction column, followed by a size-exclusion column).

In some embodiments, the IgA protease produced according to the method is a bacterial IgA protease. In certain embodiments, the bacterial IgA protease is selected from the group consisting of Haemophilus influenza IgA proteases, Neisseria gonorrhoeae IgA proteases, and Neisseria meningitidis IgA proteases. In further embodiments, the IgA protease produced according to the method is an IgA1 protease. In some embodiments, the IgA1 protease is a bacterial IgA1 protease. In certain embodiments, the bacterial IgA1 protease is selected from the group consisting of Haemophilus influenza type1 and type 2 IgA1 proteases, Neisseria gonorrhoeae type 1 and type 2 IgA1 proteases, and Neisseria meningitidis type 1 and type 2 IgA1 proteases.

In some embodiments, the IgA protease is at least about 40%, 45%, 50%, 55%, 60%, 65%, 70%, 75%, 80%, 85%, 90%, 95% or 100% identical to SEQ ID NO: 1, 2, 3, 4, 5, 6, 7, 8, 9, 10, 11, 12, 22 or 23. In an embodiment, the IgA protease is at least about 60% identical to SEQ ID NO: 1, 2, 3, 4, 5, 6, 7, 8, 9, 10, 11, 12, 22 or 23.

In further embodiments, the host cell is a bacterial host cell. In some embodiments, the bacterial host cell is selected from the group consisting of E. coli, Bacillus, Streptomyces, and Salmonella strains and cell lines. In certain embodiments, the E. coli strains and cell lines are selected from the group consisting of BL21(DE3), BL21(DE3)pLysS, BL21(DE3)pGro7, ArcticExpress, ArcticExpress(DE3), C41(DE3), C43(DE3), Origami B, Origami B(DE3), Origami B(DE3)pLysS, KRX, and Tuner(DE3). In certain embodiments, the host cell is E. coli BL21(DE3) or C41(DE3).

In some embodiments, the host cell is grown in a volume of culture media of at least about 10, 20, 30, 40, 50, 60, 70, 80, 90 or 100 liters. In certain embodiments, the host cell is grown in a volume of culture media of at least about 10 liters or 50 liters.

In additional embodiments, the host cell is grown for a time period at a temperature from about 10° C. to about 40° C. In some embodiments, the host cell is grown for a time period at about 10, 12, 15, 20, 22, 25, 26, 27, 28, 30, 35, 37 or 40° C. In certain embodiments, the host cell is grown for a time period at about 20° C., 28° C., 30° C., 35° C. or 37° C.

In other embodiments, expression of the polynucleotide is enhanced using an isopropyl β-D-1-thiogalactopyranoside (IPTG)-inducible vector. In some embodiments, the host cell is grown for a time period at a temperature from about 10° C. to about 40° C. when cultured with IPTG. In certain embodiments, the host cell is grown for a time period at about 10, 12, 15, 20, 22, 25, 26, 27, 28, 30, 35, 37 or 40° C. when cultured with IPTG. In certain embodiments, the host cell is grown for a time period at about 20° C., 28° C., 30° C., 35° C. or 37° C. when cultured with IPTG.

In still other embodiments, the host cell is cultured with IPTG at a concentration from about 0.1 mM or 0.2 mM to about 2 mM, or from about 0.2 mM or 0.4 mM to about 1 mM. In some embodiments, the IPTG is at a concentration of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9 or 2 mM. In certain embodiments, the IPTG is at about 0.4 mM or about 1 mM.

In further embodiments, the vector is a plasmid. In certain embodiments, the plasmid is selected from the group consisting of pET21a, pColdIV, pJexpress401, pHT01, pHT43, and pIBEX. In other embodiments, the plasmid comprises a promoter. In certain embodiments, the promoter is selected from the group consisting of a T7 promoter, a T5 promoter, a cold shock promoter, and a pTAC promoter.

In additional embodiments, the polynucleotide further encodes a signal peptide. In certain embodiments, the signal peptide is an IgA protease signal peptide. In other embodiments, the signal peptide is a heterologous signal peptide.

Further embodiments of the disclosure relate to a host cell comprising a vector, the vector comprising a polynucleotide encoding a serine-type IgA protease polypeptide that comprises an IgA protease proteolytic domain and lacks an α protein domain and a β-core domain, wherein the IgA protease polypeptide is expressed in/from the host cell as inclusion bodies, or as a soluble polypeptide that exhibits IgA protease activity, or a combination thereof.

In certain embodiments, the IgA protease polypeptide expressed in/from the host cell lacks at least about 40%, 50%, 60%, 70%, 80%, 90%, 95% or 100% of the α protein domain, and lacks at least about 40%, 50%, 60%, 70%, 80%, 90%, 95% or 100% of the β-core domain, or any combination of the aforementioned percentages. In certain embodiments, the IgA protease polypeptide lacks at least about 50% of the α protein domain and at least about 50% of the β-core domain. In an embodiment, the IgA protease polypeptide lacks 100% of the α protein domain and 100% of the β-core domain.

In some embodiments, the IgA protease expressed in/from the host cell is a bacterial IgA protease. In certain embodiments, the bacterial IgA protease is selected from the group consisting of Haemophilus influenza IgA proteases, Neisseria gonorrhoeae IgA proteases, and Neisseria meningitidis IgA proteases. In further embodiments, the IgA protease expressed in/from the host cell is an IgA1 protease. In some embodiments, the IgA1 protease is a bacterial IgA1 protease. In certain embodiments, the bacterial IgA1 protease is selected from the group consisting of Haemophilus influenza type1 and type 2 IgA1 proteases, Neisseria gonorrhoeae type1 and type 2 IgA1 proteases, and Neisseria meningitidis type1 and type 2 IgA1 proteases.

In some embodiments, the IgA protease is at least about 40%, 45%, 50%, 55%, 60%, 65%, 70%, 75%, 80%, 85%, 90%, 95% or 100% identical to SEQ ID NO: 1, 2, 3, 4, 5, 6, 7, 8, 9, 10, 11, 12, 22 or 23. In an embodiment, the IgA protease is at least about 60% identical to SEQ ID NO: 1, 2, 3, 4, 5, 6, 7, 8, 9, 10, 11, 12, 22 or 23.

In additional embodiments, the host cell is a bacterial host cell. In some embodiments, the bacterial host cell is selected from the group consisting of E. coli, Bacillus, Streptomyces, and Salmonella strains and cell lines. In certain embodiments, the E. coli strains and cell lines are selected from the group consisting of BL21(DE3), BL21(DE3)pLysS, BL21(DE3)pGro7, ArcticExpress, ArcticExpress(DE3), C41(DE3), C43(DE3), Origami B, Origami B(DE3), Origami B(DE3)pLysS, KRX, and Tuner(DE3). In certain embodiments, the host cell is E. coli BL21(DE3) or C41(DE3).

In other embodiments, the vector is a plasmid. In certain embodiments, the plasmid is selected from the group consisting of pET21a, pColdIV, pJexpress401, pHT01, pHT43, and pIBEX.

Additional embodiments of the disclosure relate to a composition comprising at least about 50, 60, 70, 75, 80, 90 or 100 grams wet weight of any of the host cells described herein. In certain embodiments, the composition comprises at least about 50 grams or 75 grams wet weight of the host cell.

Further embodiments of the disclosure relate to a pharmaceutical composition comprising a serine-type IgA protease produced from a host cell according to the method described above, and one or more pharmaceutically acceptable excipients, diluents and/or carriers. In some embodiments, the IgA protease is a bacterial IgA protease. In certain embodiments, the bacterial IgA protease is selected from the group consisting of Haemophilus influenza IgA proteases, Neisseria gonorrhoeae IgA proteases, and Neisseria meningitidis IgA proteases. In additional embodiments, the IgA protease is an IgA1 protease. In some embodiments, the IgA1 protease is a bacterial IgA1 protease. In certain embodiments, the bacterial IgA1 protease is selected from the group consisting of Haemophilus influenza type1 and type 2 IgA1 proteases, Neisseria gonorrhoeae type1 and type 2 IgA1 proteases, and Neisseria meningitidis type1 and type 2 IgA1 proteases. In further embodiments, the IgA protease is at least about 40%, 45%, 50%, 55%, 60%, 65%, 70%, 75%, 80%, 85%, 90%, 95% or 100% identical to SEQ ID NO: 1, 2, 3, 4, 5, 6, 7, 8, 9, 10, 11, 12, 22 or 23. In an embodiment, the IgA protease is at least about 60% identical to SEQ ID NO: 1, 2, 3, 4, 5, 6, 7, 8, 9, 10, 11, 12, 22 or 23.

In addition, all the embodiments described elsewhere in the present disclosure, including without limitation the Summary, are representative embodiments of the disclosure. Additional embodiments and details of the disclosure will be apparent from the following examples, which are intended to be illustrative rather than limiting.

### EXAMPLES

### Example 1

### Production of Soluble, Active IgA1 Protease in Different E. coli Strains

Production of recombinant IgA protease in amounts sufficient for therapeutic use has not been achieved due to the low natural production of the protein in naturally producing cells such as H. influenzae, N gonorrohoeae, and N. meningitidis. Moreover, previous attempts at production of IgA protease produced low titers of total protein, and also did not produce significant amounts of soluble protein isolatable directly from the cell culture media or supernatant (see, e.g., Khomenkov et al., Mol. Genetics, Microbiol. and Virol., 22:34-40 (2007); Grundy et al., J. Bacteriol., 169:4442-50 (1987); U.S. Pat. No. 5,965,424; and Vitovski et al., Infect. Immun., 75:2875-85 (2007)).

To improve recombinant production of an IgA protease (e.g., IgA1 protease), four IgA1 protease expression constructs were generated for expression of IgA1 protease in E. coli. ELISA and IgA1 protease activity assays were developed for screening the expression, solubility and activity of IgA1 protease. The expression of soluble IgA1 proteases in different cell strains, at different temperatures and at different concentrations of inducible agent, such as isopropyl β-D-1-thiogalactopyranoside (IPTG), was screened.

### Materials and Methods

Cloning of His-Tagged IgA1 Protease into Expression Vectors

IgA1 protease fragments for each construct were amplified from pFG26 plasmid (from IGAN Biotech, containing full-length wild-type H. influenzae IgA1 protease gene) by PCR using a different pair of primers (FIG. 1 and Table 1). Amplified PCR fragments were digested with Nde I and BamHI and cloned into pET21a (Novagen, Gibbstown, N.J.), pColdIV (Takara, Shiga, Japan) and pJexpress401 (DNA2.0, Menlo Park, Calif.) vectors (Table 1). A construct with a signal peptide is designated S-IGAN.

TABLE 1
IgA1 protease expression constructs

| | | | | Restriction | |
|---|---|---|---|---|---|
| Construct | Vector | Primer | Tag | Enzyme | Expression |
| pET-S- | pET21a | IgA-NdeI-SS-5′: | C- | Nde I | E. coli |
| IGAN | | Gctcatatgctaaataaaaaattcaaactc | terminal | BamHI | periplasm |
| (pET21a- | | (SEQ ID NO. 13) | hexa- | | |
| S-IgA-his) | | IgA-6his-BamHI-3′: | His tag | | |
| | | caaggatcctaggtggtggtggtggtggt | | | |
| | | gaggcacatcagcttgaatattattag | | | |
| | | (SEQ ID NO. 14) | | | |
| pET- | pET21a | IgA-NdeI-5′: | C- | Nde I | E. coli |
| IGAN | | gctcatatggcgttagtgagagacgatgtg | terminal | BamHI | cytoplasm |
| (pET21a- | | (SEQ ID NO. 15) | hexa- | | |
| IgA-his) | | IgA-6his-BamHI-3′: | His tag | | |
| | | caaggatcctaggtggtggtggtggtggt | | | |
| | | (SEQ ID NO. 16) | | | |
| | | gaggcacatcagcttgaatattattag | | | |
| | | (SEQ ID NO. 17) | | | |
| pCold-S- | pColdIV | IgA-NdeI-SS-5′: | C- | Nde I | E. coli |
| IGAN | | gctcatatgctaaataaaaaattcaaactc | terminal | BamHI | periplasm |
| (pColdIV- | | (SEQ ID NO. 18) | hexa- | | |
| S-IgA-his) | | IgA-6his-BamHI-3′: | His tag | | |
| | | caaggatcctaggtggtggtggtggtggt | | | |
| | | gaggcacatcagcttgaatattattag | | | |
| | | (SEQ ID NO. 19) | | | |
| pCold- | pColdIV | IgA-NdeI-5′: | C- | Nde I | E. coli |
| IGAN | | gctcatatggcgttagtgagagacgatgtg | terminal | BamHI | cytoplasm |
| (pColdIV- | | (SEQ ID NO. 20) | hexa- | | |
| IgA-his) | | IgA-6his-BamHI-3′: | His tag | | |
| | | caaggatcctaggtggtggtggtggtggt | | | |
| | | gaggcacatcagcttgaatattattag | | | |
| | | (SEQ ID NO. 21) | | | |
| pJEX-IgA | pJexpress401 | IgA-NdeI-5′: | No tag | Nde I | E. coli |
| | | gctcatatggcgttagtgagagacgatgtg | | BamHI | cytoplasm |
| | | (SEQ ID NO. 20) | | | |
| | | IgA-6his-BamHI-3′: | | | |
| | | caaggatcctaaggcacatcagcttgaata | | | |
| | | ttattag | | | |
| | | (SEQ ID NO. 24) | | | |

Expression of His-Tagged IgA1 Protease in E. coli

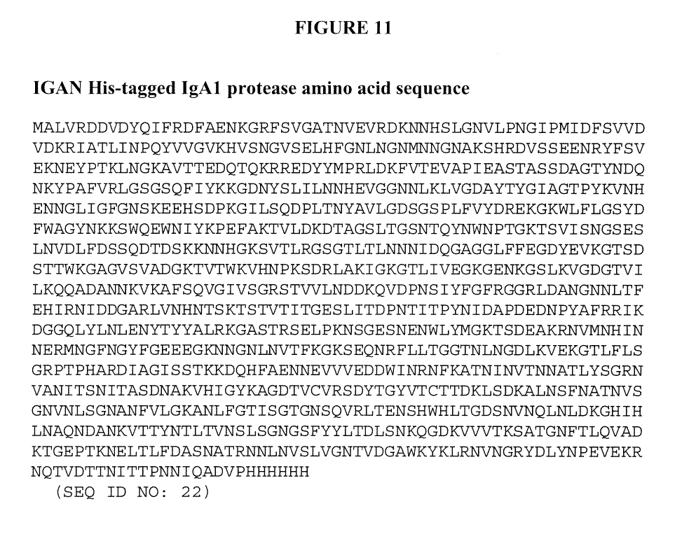

pET21a and pCold plasmids expressing C-terminal His-tagged H. influenzae IgA1 protease (SEQ ID NO: 22) (FIG. 11) were transformed into various E. coli strains [BL21(DE3), BL21(DE3)pLysS, BL21(DE3)pGro7, ArcticExpress(DE3), C41(DE3) (or C41), C43(DE3) (or C43), C41pLysS, C43pLysS, Origami B(DE3), Origami B(DE3)pLysS, KRX and Tuner(DE3)] (Table 2). Transformed cells were plated on LB plates containing 100 ug/mL carbeniciline and incubated overnight at 37° C. One single colony was picked and cultured in 4 mL LB medium containing 100 ug/mL of carbeniciline at 37° C. with shaking. When an OD₆₀₀ of bacterial culture reached 0.6, 0.2-1.0 mM isopropyl β-D-1-thiogalactopyranoside (IPTG) was added to the cell media and incubated at 12° C. to 30° C. for 3-24 hours with shaking. For cell harvest, bacterial cells were centrifuged and cell pellets lysed with B-PER II Bacterial Extraction Reagent (PIERCE, 1 mL per 4 mL of bacterial culture).

Bacterial crude extract was reserved and centrifuged to obtain supernatant. Supernatant and crude extract were assayed for IgA1 protease expression and solubility by ELISA, SDS-PAGE and Western blot.

TABLE 2
E. coli strains for IgA protease (e.g., IgA1 protease) expression

| | Supply | |
|---|---|---|
| E. coli Strain | Company | Description |
| BL21(DE3) | Stratagene | Used with pET vector; encodes T7 RNA |
| | | polymerase under the control of the lacUV5 |
| | | promoter; general expression host |
| BL21(DE3)pLysS | Stratagene | BL21(DE3) with pLysS plasmid that codes |
| | | for T7 lysozyme, a T7 RNA polymerase |
| | | inhibitor; high-stringency expression |
| BL21Star ™(DE3) | Invitrogen | BL21(DE3) containing a mutation in the |
| | | gene encoding RNaseE (rne131); |
| | | significantly improves the stability of mRNA |
| | | transcripts and increases protein expression |
| | | yield from T7 promoter-based vectors |
| BL21Star ™(DE3)pLysS | Invitrogen | BL21Star ™(DE3) with pLysS plasmid |
| BL21(DE3)pGro7 | Takara | Co-expresses chaperone groES-groEL, |
| | | enhancing protein folding and solubility |
| BL21(DE3)pGro7/pG- | Takara | BL21(DE3) with plasmids to co-express |
| KJE8/pKJE7/pG- | | chaperones groES-groEL, dnaK-dnaJ-grpE |
| Tf2/pTf16 | | and tig, enhancing protein folding and |
| | | solubility |
| ArcticExpress(DE3) | Stratagene | Co-expresses the cold-adapted chaperonins |
| | | Cpn10 and Cpn60, enhancing protein folding |
| | | and solubility |
| C41 (aka C41(DE3)) | Lucigen | BL21(DE3) with uncharacterized mutations; |
| | | toxic protein expression |
| C41(DE3)pLysS | Lucigen | C41(DE3) with pLysS plasmid |
| C43 (aka C43(DE3)) | Lucigen | C41(DE3) with uncharacterized mutations; |
| | | toxic protein expression |
| C43(DE3)pLysS | Lucigen | C43(DE3) with pLysS plasmid |
| Origami B(DE3) | Novagen | K-12 with TrxB and Gor mutations in |
| | | cytoplasmic disulfide reduction pathway, |
| | | enhancing disulfide bond formation |
| Origami B(DE3)pLysS | Novagen | Origami B(DE3) with pLysS plasmid; high- |
| | | stringency expression; enhances disulfide |
| | | bond formation |
| KRX | Promega | K-12 that contains T7 RNA polymerase |
| | | under the control of a rhamnose promoter; |
| | | high-stringency expression |
| Tuner(DE3) | Novagen | BL21(DE3) with lac permease mutation; |
| | | allows control of expression level |
| Tuner(DE3)pLysS | Novagen | Tuner(DE3) with pLysS plasmid |
| SHuffle ™ T7 express | NEB | BL21(DE3) strain lacks the two reductases |
| | | (trxB and gor) with an additional suppressor |
| | | mutation (ahpC) that restores viability, |
| | | allowing for the formation of stable disulfide |
| | | bonds in the cytoplasm; expresses the |
| | | disulfide bond isomerase DsbC within the |
| | | cytoplasm and enhances the fidelity of |
| | | disulfide bond formation in the cytoplasm |
| SHuffle ™ T7 express | NEB | SHuffle ™ T7 express strain contains inactive |
| lysY | | mutant lysozyme expressed from miniF and |
| | | allows for the expression of toxic proteins |

Detection of His-Tagged IgA1 Protease Expression with Western Blot

Ten uL of cell lysates or soluble supernatants was run on sodium dodecyl sulfate polyacrylimide gel electrophoresis (SDS-PAGE), and the protein was transferred to membrane with gel blot (Invitrogen, Carlsbad, Calif.). The membrane was blocked in TBS buffer with 5% milk at room temperature (RT) for 1 hour. Rabbit anti-His polyclonal Ab (1:2500 dilution) was added and incubated at RT with shaking for 2 hours, and the membrane was washed 3 times with TBS buffer.

Alkaline phosphate (AP) conjugated anti-rabbit IgG (1:5000 dilution) was added to the membrane and incubated at RT with shaking for 1 hour, and the membrane was washed 3 times with TBS buffer. Ten mL of WESTERN BLUE® Stabilized Substrate (Promega, Madison, Wis.) was then added and incubated at RT with shaking for 1 to 5 min, and the membrane was washed with TBS buffer to remove excess stain.

IgA1 Protease Activity Assay with Western Blot

Ten uL of cell lysates or soluble supernatants was mixed with 10 uL of IgA1 (10 ug) and incubated at 37° C. overnight. Ten uL of cleaved products was run on SDS-PAGE, the protein was transferred to membrane with Gel blot (Invitrogen), and the membrane was blocked in TBS buffer with 5% milk at RT for 1 hour. Mouse anti-IgA-F_{ab} monoclonal antibody (mAb) (1:2500 dilution) was added and incubated at RT with shaking for 2 hours, and the membrane was washed 3 times with TBS buffer.

Alkaline phosphate (AP) conjugated anti-rabbit IgG (1:5000 dilution) was added to the membrane and incubated at RT with shaking for 1 hour, and the membrane was washed 3 times with TBS buffer. Ten mL of WESTERN BLUE® Stabilized Substrate (Promega) was then added and incubated at RT with shaking for 1 to 5 min, and the membrane was washed with TBS buffer to remove excess stain.

### Screening Soluble His-Tagged IgA1 Protease Expression by ELISA

The purified His-tagged IgA1 protease (10 ug, 1.0 ug, 0 ug) was diluted in Binding Solution (bacterial crude extract or supernatant). 100 uL of diluted His-tagged protein as well as 100 uL of IgA1 protease samples were added to the wells of an enhanced protein-binding ELISA plate (Nunc MAXISORP™, Rochester, N.Y.), and then the plate was sealed to prevent evaporation and incubated overnight at 4° C.

The plate was allowed to warm to RT, the binding solution was removed by washing 3 times with 200 uL of PBST (PBS+0.05% Tween-20), and the plate was blocked against non-specific binding by adding 100 uL of Blocking Buffer (PBST+3% BSA). The plate was sealed and incubated at RT for 1-2 hr.

Blocking Buffer was removed by washing 3 times with PBST. Anti-His antibody (Abcam) was diluted in Blocking Buffer to 0.3 ug/mL (1:3,000 dilution), 100 uL of diluted antibody was added to each well, and the plate was sealed and incubated for 2-3 hr at RT.

Anti-His antibody solution was removed and the plate was washed 4 times with PBST. Dilute horseradish peroxidase (HRP) conjugated anti rabbit IgG H&L (Abcam, Cambridge, Mass.) was diluted in Blocking Buffer to 0.1 ug/mL (1:10,000 dilution), 100 uL of diluted antibody was added to each well, and the plate was sealed and incubated for 30 min to 1 hr at RT.

HRP conjugated anti rabbit IgG H&L solution was removed and the plate was washed 4 times with PBST. 100 uL of 1-Step Turbo TMB-ELISA (Pierce, Rockford, Ill.) was added to each well and incubated for 5-30 min at RT. The reaction was quenched by addition of 100 uL of Stop Solution (1-2 M sulfuric acid), and then absorbance at 450 nm was measured.

Expression of His-Tagged IgA1 Protease in E. coli C41(DE3) Cells

Cells [E. coli strain C41(DE3) (or C41), expression of pET-IGAN construct (or pET21a-IgA-his)] from glycerol stock stored at −80° C. were grown in 2 mL LB medium containing 100 ug/mL of carbenicillin at 37° C. overnight with shaking (250 rpm) for 1 day. On day 2, 40 uL of overnight grown cell culture was transferred to 4 mL LB medium containing 100 ug/mL of carbenicillin and grown at 37° C. with shaking (250 rpm). When the OD₆₀₀ reached 0.6, the cell culture was transferred to 12° C. and incubated for 20 minutes. IPTG was then added to a final concentration of 0.4 mM to induce protein expression at 20° C. with shaking (250 rpm) for 24 hours. Cells were then spun down, and the resulting cell pellet was either lysed or frozen at −80° C.

### Results

### Cloning of His-Tagged IgA1 Protease

The serine-type IgA1 protease is initially translated as a precursor protein comprising a signal peptide that targets it to the periplasm, the mature protease domain, the α-protein domain that is secreted with the mature protease domain, and the β-core domain that transports the protease across the outer membrane. The β-core domain integrates into the outer membrane and forms a specific pore via which the mature protease domain and the α-protein domain are translocated through the periplasm into the extracellular space. The mature IgA1 protease is released by self-cleavage at three cleavage sites: a, b and c (FIG. 1). The IgA1 protease domain with or without the signal peptide, and lacking the entirety of both the α-protein domain and the β-core domain, was cloned into a pET21a expression vector (T7 promoter) and a pColdIV expression vector (cold shock promoter, expression occurs at low temperature (<15° C.)).

Soluble and Active His-Tagged IgA1 Proteases were Expressed in E. coli

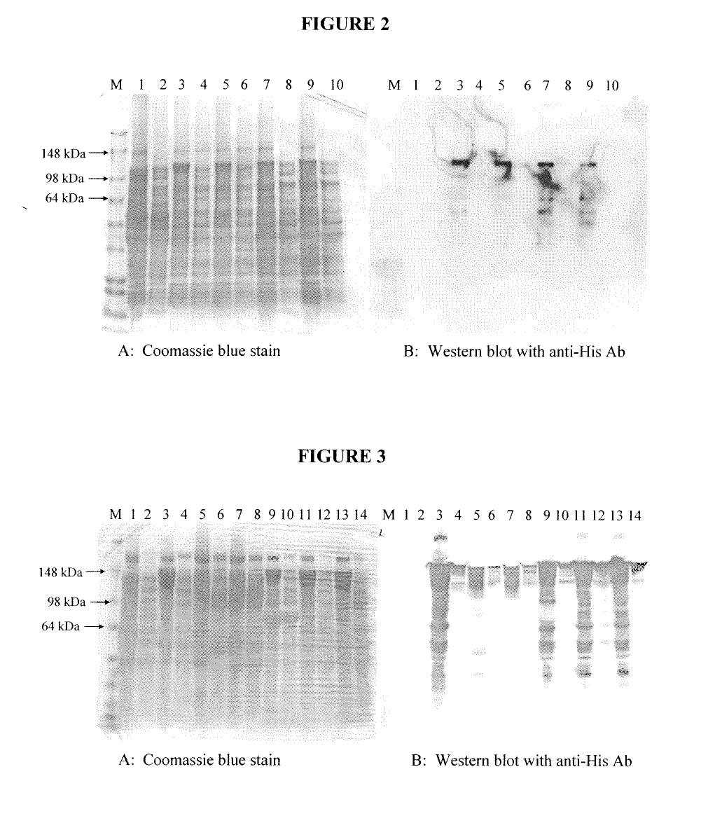

pET-S-IGAN and pET-IGAN were first expressed in BL21(DE3) cells induced with 1 mM IPTG at 30° C. for 3 hours. The presence of IgA1 protease was assayed in both the cell lysate (FIG. 2, lanes 1, 3, 5, 7 and 9) and the cell supernatant (Lanes 2, 4, 6, 8, and 10). FIG. 2 shows that both constructs expressed IGAN IgA1 proteases as inclusion bodies and not as soluble material. When the expression of pET-S-IGAN and pET-IGAN was induced at low temperature (12° C.) and low amount of IPTG (0.4 mM) in different cell strains (BL21(DE3), C41(DE3), C43(DE3), BL21(DE3)pGro7, Origami B(DE3), Origami B(DE3)pLysS), small fractions of expressed IgA1 proteases were soluble, as evidenced by the detection of IgA1 protease in the cell lysate (FIG. 3).

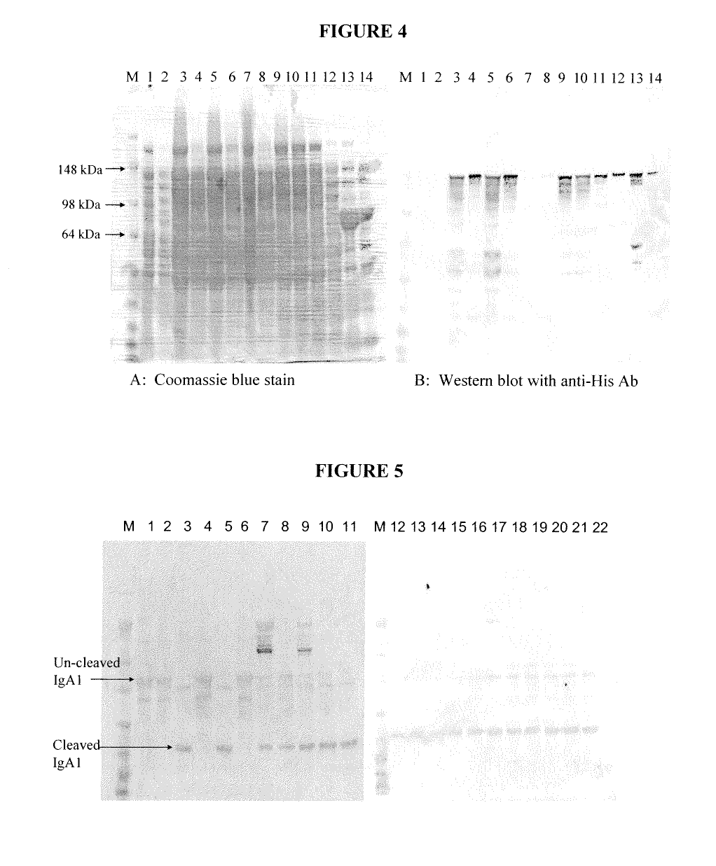

When the expression of pCold-S-IGAN and pCold-IGAN was induced at low temperature (15° C.) and low amount of IPTG (0.4 mM) in different cell strains (BL21(DE3), C41(DE3), C43(DE3), BL21(DE3)pLysS, Origami B(DE3), BL21(DE3)pGro7), all expressed IgA1 proteases were soluble, but in lower overall titer (FIG. 4). C43(DE3) cells did not show any IgA1 protease expression in this assay. The IgA1 proteases expressed from the four constructs and in different E. coli cells were tested for IgA1 cleavage activity by Western blot. All the expressed IgA1 proteases exhibited IgA1 cleavage activity (FIG. 5).

Screening Soluble His-Tagged IgA1 Protease Expression with ELISA

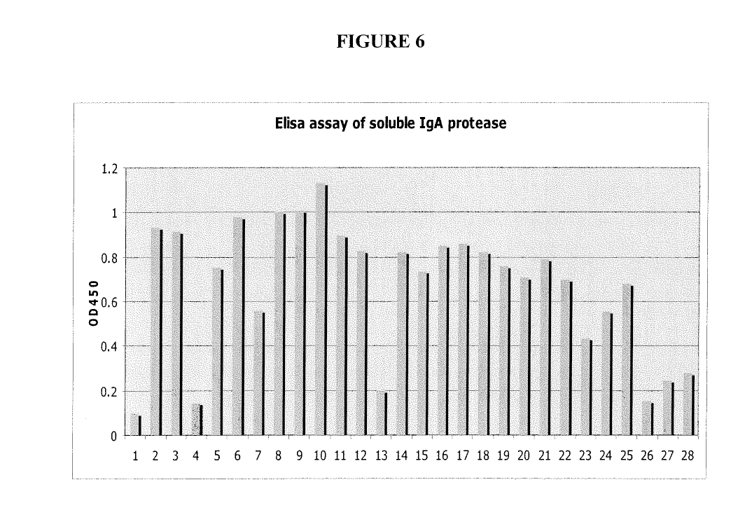

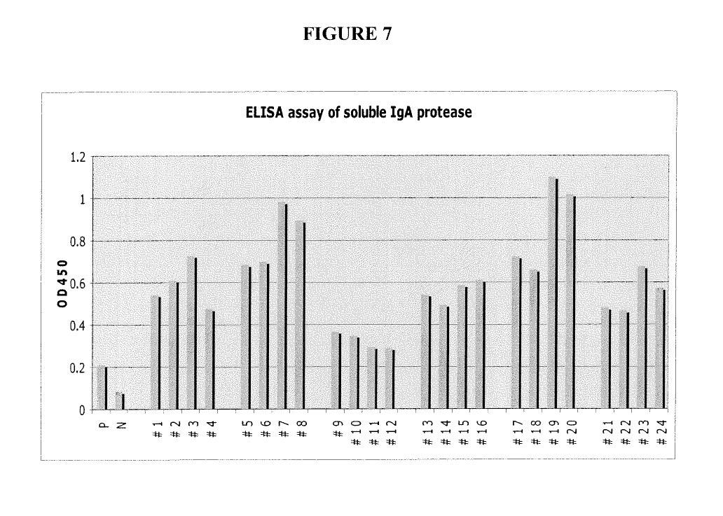

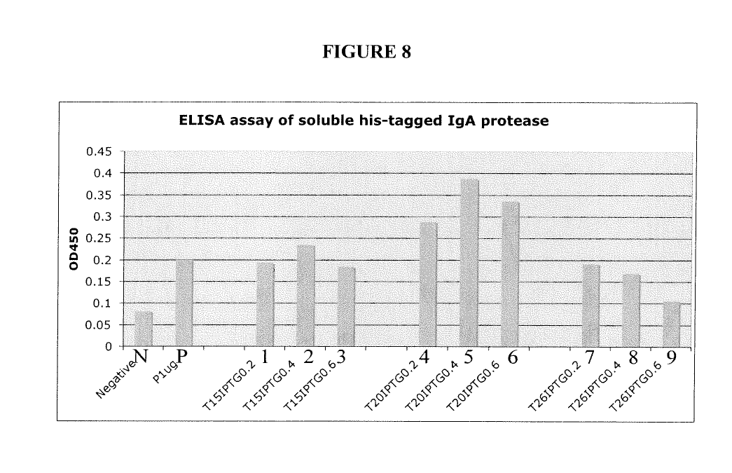

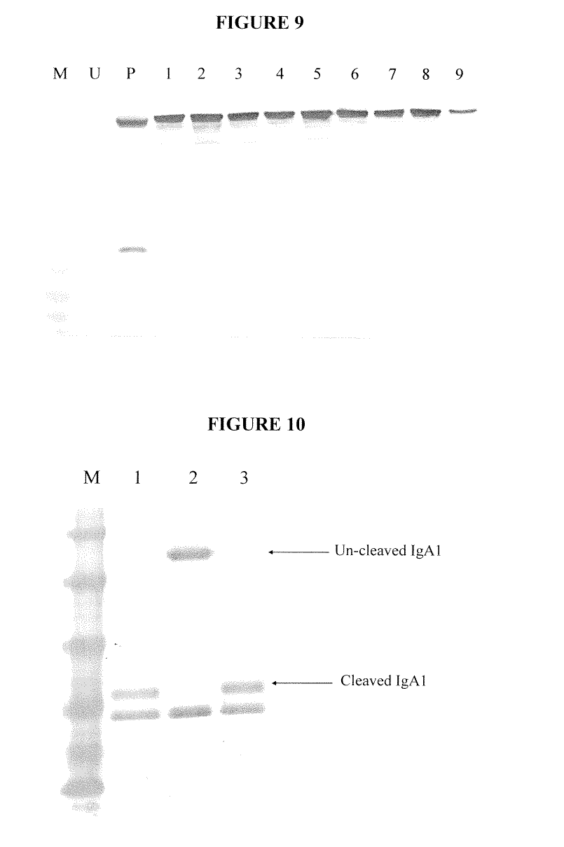

The levels of IgA1 proteases expressed by all four IgA1 protease constructs described above (pET-S-IGAN, pET-IGAN, pCold-S-IGAN and pCold-IGAN), whose expressions were induced at low temperature (12° C.) and low amount of IPTG (0.4 mM) in different cell strains, were screened by ELISA assay. The pET-IGAN construct resulted in the production of greater levels of soluble IgA1 protease in several cell strains (FIG. 6: pET-S-IGAN: samples 2-10, pET-IGAN: samples 11-18, pCold-S-IGAN: samples 19-23 and pCold-IGAN: samples 24-28). Overall, greater amounts of soluble IgA1 protease were produced at 20° C. and 0.4 mM IPTG in most cell strains. The C41(DE3) strain produced the highest titer of IgA1 protease under the same conditions compared with other E. coli strains used for recombinant expression (FIG. 7, samples 17-20). Soluble IgA1 protease in C41(DE3) cells grown at different temperatures and IPTG concentrations was detectable by ELISA at all culture conditions (FIG. 8) and confirmed by Western blot (FIG. 9). In the screening studies, the C41(DE3) E. coli strain containing the pET-IGAN plasmid, when induced with 0.4 mM IPTG at 20° C. for 24 hours, produced the highest level of soluble IgA1 protease (about 20-40 mg/L) among the cell strains and conditions tested. IgA1 protease produced from C41(DE3) cells also showed IgA1 cleavage activity (FIG. 10).

The results described above demonstrate that all four IgA1 protease expression constructs (pET-S-IGAN, pET-IGAN, pCold-S-IGAN and pCold-IGAN) encoding IgA1 protease polypeptides that comprise the proteolytic protease domain, with or without the signal peptide, and lack the entirety of both the α protein domain and the β-core domain were able to produce soluble and active IgA1 proteases in several E. coli strains when induced at low temperature and low concentration of IPTG. ELISA and IgA1 protease activity assay showed that soluble, active IgA1 proteases were expressed in several different E. coli strains over a range of culture temperatures and IPTG concentrations. In the screening studies, E. coli C41(DE3) cells transformed with the pET-IGAN construct produced the highest titer (approximately 20-40 mg/L) of soluble, active IgA1 protease when induced with 0.4 mM IPTG at 20° C.

### Example 2

### Direct Production of Soluble, Active IgA1 Protease in E. coli C41(DE3) Cells

Soluble, active Haemophilus influenzae IgA1 protease containing the proteolytic protease domain and lacking the α protein and β-core domains was directly produced in E. coli C41(DE3) cells. Briefly, the IgA1 protease was recombinantly expressed in C41(DE3) cells, and the cells were harvested. The cells were suspended in 1×TBS and lysed by a high-pressure homogenizer to release soluble IgA1 protease from the cells. The homogenized suspension was centrifuged, and the supernatant containing soluble IgA1 protease was filtered. If the resulting pellet contains IgA1 protease inclusion bodies, the pellet can be saved for solubilization and refolding of the inclusion bodies. The soluble IgA1 protease was purified by a nickel column, an anion-exchange column, and a size-exclusion column.

### 1. Expression of IgA1 Protease

H. influenzae IgA1 protease containing the proteolytic protease domain and lacking the α protein and β-core domains was expressed in E. coli C41(DE3) cells grown in a fermenter. The fermentation was conducted at a lower temperature, 20° C., to promote formation of soluble IgA1 protease rather than insoluble IgA1 protease inclusion bodies. The type of E. coli host cell [C41(DE3)] and the lower fermentation temperature (20° C.) were chosen to promote formation of soluble IgA1 protease, but might have resulted in lower total yield of IgA1 protease.

A portion (0.2 mL) of an overnight seed culture containing C41(DE3) cells transformed with the pET-IGAN expression construct was added to a flask containing 500 mL of LB medium (Luria broth, pH 7.0) at 37° C. The seed flask was incubated at 37° C. and agitated at 225 rpm until the culture reached a cell density between 2.0 and 4.0 OD₆₀₀. Ampicilline (50 mg/L) was added to the seed flask medium just before the whole medium was transferred to a 20 L fermenter containing 17 L of a fermenter medium (680 mL glycerol, 408 g yeast extract, 204 g tryptone, 170 g casamino acids, 15 mL polypropylene glycol Pluracol® P2000 (BASF), 1.7 L 1 M MOPS [3-(N-morpholino)propanesulfonic acid], 5 N NaOH, 85% H₃PO₄, pH 7.2).

The batch phase of fermentation was conducted at a temperature of 37° C., a pH of 7.2, and a dissolved oxygen concentration of 30%. When the cell density of the culture medium reached 10.9 OD₆₀₀, the medium was cooled to 20° C. Induction of IgA1 protease expression was initiated by the addition of isopropyl β-D-1-thiogalactopyranoside (IPTG) to a final IPTG concentration of 1 mM. The induction phase of fermentation proceeded at 20° C. until the cell density reached 31.2 OD₆₀₀).

When the cell density reached 31.2 OD₆₀₀, fermentation was ended to minimize or avoid the formation of insoluble IgA1 protease. The cells were harvested and centrifuged, and the resulting cell pellet (87.7 g/L wcw (wet cell weight)) was stored at −80° C.

### 2. Isolation of Soluble IgA1 Protease

The crude cell pellet was thawed, and then was suspended and mixed for 20 min in a buffer (25 mM Na₂PO₄, 150 mM NaCl, 20 mM imidazole, pH 6.8), to a volume of 750 mL buffer for every 60 g wet cell pellet. The cells were homogenized by passing the cells four times through a homogenizer at 8000 psi, which lysed the cells and released soluble IgA1 protease and contaminants (e.g., DNA, lipids, non-specific proteins) from the cells, and the resulting sample was collected on ice. The sample was centrifuged at 10,000 g for 30 min, and the supernatant containing soluble IgA1 protease and contaminants was collected and filtered through 0.45 micron and 0.2 micron filters (Sartorius, Aubagne, France) for purification of the IgA1 protease. If the resulting pellet contains IgA1 protease inclusion bodies, the pellet can be saved for solubilization and refolding of the inclusion bodies (see Examples 3 and 4).

### 3. Purification of Soluble IgA1 Protease

The filtered solution containing soluble IgA1 protease was loaded onto a Nickel IMAC Chelating Sepharose column (CV=42.5 mL, 8.0 cm×2.6 cm, GE Healthcare) charged with 50 mM NiSO₄ at a flow rate of 57 cm/hr. The column was washed with an equilibration buffer (25 mM Na₂PO₄, 150 mM NaCl, 20 mM imidazole, pH 6.8) at a flow rate of 113 cm/hr, during which the His-tagged protease bound to the nickel column. The His-tagged IgA1 protease was eluted off the nickel column by elution with increasing concentrations of imidazole (to 25 mM Na₂PO₄, 150 mM NaCl, 250 mM imidazole, pH 6.8) at a flow rate of 113 cm/hr. Fractions containing the main eluate product peak were combined, sterile-filtered, and diluted 10-fold with an equilibration buffer of a Q sepharose column (25 mM Tris, pH 8.0).

The diluted solution containing the main eluate product from the nickel column was loaded onto a Q Sepharose FF anion-exchange column (CV=14.8 mL, GE Healthcare) at a flow rate of 150 cm/hr, and the column was washed with the equilibration buffer (25 mM Tris, pH 8.0) at a flow rate of 150 cm/hr. Soluble, unaggregated IgA1 protease did not bind to the column and was collected in the flow-through. Contaminants and IgA1 protease aggregates bound to the column and were eluted off the column using an elution buffer (25 mM Tris, 1 M NaCl, pH 8.0) at a flow rate of 150 cm/hr. Flow-through fractions containing the main unbound product peak were combined, sterile-filtered and concentrated to around 20-35 mL by tangential flow filtration (Vivaflow 200, 30 kDa PES, Sartorius).

Recovery and purity of soluble IgA1 protease may be increased in various ways—e.g., optimization of the Q sepharose chromatography, non-use of the Q sepharose column, replacement of the Q sepharose column with other column(s) (e.g., with an anion-exchange column, such as a GigaCap Q column (Tosoh BioSciences), and/or with a hydrophobic-interaction column, such as a butyl sepharose 4 column (GE Healthcare)), etc.

The concentrated solution containing the main unbound product from the flow-through of the Q sepharose column was loaded onto an S300 Sephacryl HR size-exclusion column (CV=1.8765×95 cm, GE Healthcare), which purifies proteins by their different sizes. IgA1 protease was eluted off the column using a mobile phase of 1×TBS (Tris-buffered saline—25 mM Tris, 150 mM NaCl, pH 7.5) at a flow rate of 30 cm/hr, and fractions containing the main eluate product peak were combined and sterile-filtered. Chromatography with the S300 column indicated the presence of IgA1 protease aggregates and lower molecular weight contaminants, which were separated from soluble, unaggregated IgA1 protease by collection of appropriate eluate fractions.

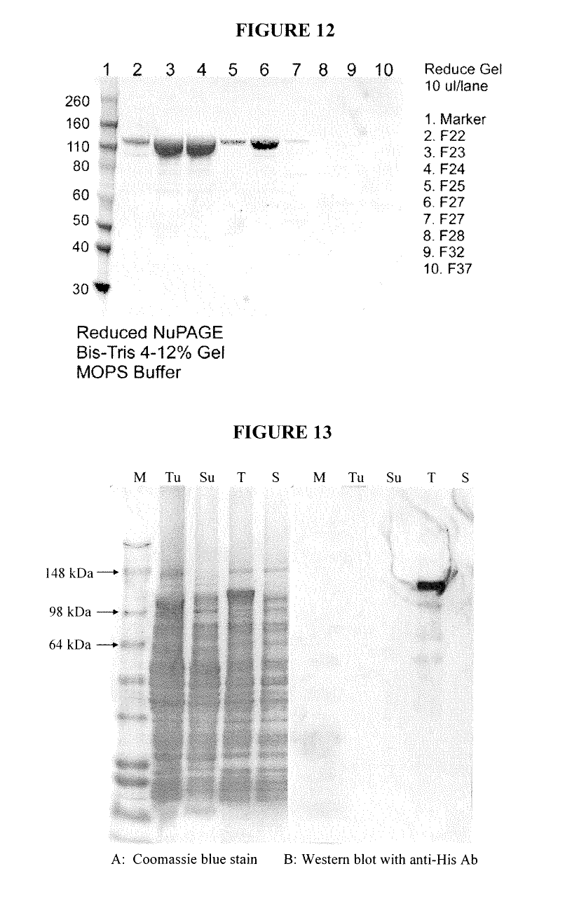

Chromatography with the S300 column provided soluble IgA1 protease of sufficiently high purity (FIG. 12; fractions 23 and 24 were collected as the final product), and in a formulation buffer (TBS), for biological testing. The purified soluble IgA1 protease can be utilized in in vitro assays (e.g., assessing cleavage of human IgA1) and in vivo studies (e.g., animal models of IgA nephropathy).

### Example 3

### Smaller-Scale Production of Active IgA1 Protease via Inclusion Bodies from BL21(DE3)

Cloning of His-Tagged IgA1 Protease into Expression Vector

DNA fragments encoding the proteolytic protease domain of Haemophilus influenzae IgA1 protease were amplified from a pFG26 plasmid (from IGAN Biotech, containing the full-length wild-type H. influenzae IgA1 protease gene) by PCR using the primers IgA-NdeI-5′ (gctcatatggcgttagtgagagacgatgtg) (SEQ ID NO:20) and IgA-6his-BamHI-3′ (caaggatcctaggtggtggtggtggtggtgaggcacatcagcttgaatattattag) (SEQ ID NO:21). The amplified PCR fragments were digested with NdeI and BamHI and cloned into a pET21a vector (Novagen).

Expression of IgA1 Protease in E. coli

The pET-IGAN construct (pET21a plasmid expressing C-terminal His-tagged IgA1 protease) was transformed into E. coli BL21(DE3) cells. The transformed cells were plated on an LB plate containing 100 ug/mL carbeniciline and incubated overnight at 37° C. One single colony was collected and cultured in 4 mL LB medium containing 100 ug/mL carbeniciline at 37° C. with shaking. When OD₆₀₀ of the bacterial culture reached 0.6, isopropyl β-D-1-thiogalactopyranoside (IPTG) was added to a final concentration of 1 mM, and the bacterial culture was incubated at 37° C. for 3 hours with shaking.

The bacterial cells were centrifuged, and the resulting cell pellet was collected and lysed with B-PER II Bacterial Extraction Reagent (PIERCE, 1 mL per 4 mL of bacterial culture) containing 1/1000 diluted Benzonase nuclease (Novagen). The crude bacterial extract was collected and centrifuged to afford a supernatant. The supernatant and crude bacterial extract were assayed for IgA1 protease expression and solubility by SDS-PAGE and Western blot.

H. influenzae His-tagged IgA1 protease containing the proteolytic protease domain and lacking the α protein and β-core domains was expressed as inclusion bodies in E. coli BL21(DE3) cells when induced with 1 mM IPTG at 37° C. for 3 hours. A Western blot using anti-His antibody confirmed the expression of IgA1 protease inclusion bodies (FIG. 13). The IgA1 protease inclusion bodies were readily purified by three rounds of washing and centrifugation (described below).

### Expression, Isolation and Purification of IgA1 Protease Inclusion Bodies

E. coli BL21(DE3) cells containing the pET-IGAN expression construct were cultured in 2 mL LB medium containing 100 ug/mL carbeniciline at 37° C. overnight with shaking (250 rpm). Two mL of the overnight bacterial culture was transferred to 200 mL LB medium containing 100 ug/mL carbeniciline, and shaking was continued at 37° C. When OD₆₀₀ of the bacterial culture reached 0.6, IPTG was added to a final concentration of 1 mM, and the culture was incubated at 37° C. for 3 hours with shaking. The bacterial culture was centrifuged at 5000 rpm for 5 min, and the resulting cell pellet was suspended in 30 mL of buffer A (50 mM Tris, 150 mM NaCl, pH 7.9).

The cell pellet suspension was sonicated on ice for 5 min (50%, 1 second, pause 2 seconds) to break the cells and release the cells' content (including soluble proteins and inclusion bodies), and then was centrifuged at 12,000 rpm and 4° C. for 20 min. The resulting pellet containing cells and IgA1 protease inclusion bodies (the majority) was suspended in 30 mL of buffer A. This sequence was repeated (3-5 times) until the supernatant became clear. The resulting IgA1 protease inclusion body pellet was stored at 4° C.

### Solubilization of Inclusion Bodies and Screening of Refolding Conditions

IgA1 protease inclusion bodies were solubilized in a solubilization buffer (50 mM Tris, 150 mM NaCl, pH 7.9) containing 4, 6 or 8 M urea, or 4, 6 or 8 M guanidine hydrochloride. (Alternatively, solubilization using a MES buffer (pH 5.8), or a CHES buffer (pH 9.5), containing 4, 6 or 8 M urea, or 4, 6 or 8 M guanidine hydrochloride, can be evaluated.) The resulting mixture was sonicated on ice for 5 min (50%, 1 second, pause 2 seconds), rocked at room temperature for one hour, and then centrifuged at 12,000 rpm and 4° C. for 20 min. The supernatant was collected, and the concentration of the solubilized inclusion bodies therein was adjusted to 2 mg/mL by addition of the solubilization buffer.

A portion (0.05 mL) of the solubilized inclusion body solution (2 mg/mL) was added to 0.95 mL of various refolding buffers (Table 3), and the resulting mixture was slowly rocked at 4° C. or room temperature overnight for refolding. Table 3 lists non-limiting parameters and conditions that can be tested for the refolding of solubilized IgA1 protease inclusion bodies, including without limitation the presence or absence of particular chemicals in the refolding buffers, different combinations of chemicals in the refolding buffers, the concentrations of chemicals used in the refolding buffers, the pH of the refolding buffers, etc.

TABLE 3
Conditions for refolding of solubilized inclusion bodies (IB)

| | MES | Tris buffer | CHES |
|---|---|---|---|
| | buffer pH 5.8 | pH 8.0 | buffer pH 9.5 |
| NaCl/KCl | | | |
| Guanidine hydrochloride | | | |
| Urea | | | |
| L-arginine | | | |
| DTT; GSH/GSSG | | | |
| EDTA | | | |
| PEG | | | |
| MgCl₂/CaCl₂ | | | |
| Glycerol | | | |
| Sucrose | | | |
| CHAPS | | | |
| Glycine | | | |
| IB solubilization with | | | |
| urea or guanidine•HCl | | | |
| IB concentration | | | |
| Refolding temperature | | | |
| Refolding by dilution | | | |
| Refolding by dialysis | | | |
| Refolding on column | | | |

The refolded IgA1 protease underwent dialysis with PBS buffer at 4° C. overnight to change the buffer to PBS.

Methods used to screen and optimize the refolding conditions and identify properly refolded IgA1 protease included HPLC-size exclusion chromatography and assay of IgA1 protease cleavage of IgA1 using the Experion automated electrophoresis system (described below).

### Isolation and Solubilization of Inclusion Bodies, Refolding of Solubilized Inclusion Bodies, and Purification of Refolded IgA1 Protease

Five mL of bacterial culture (from fermentation (see Example 4), OD₆₀₀=189) was centrifuged at 5000 rpm for 5 min, and the resulting cell pellet was suspended in 20 mL of buffer A (50 mM Tris, 150 mM NaCl, pH 7.9).

The cell pellet suspension was sonicated on ice for 5 min (2 seconds, pause 4 seconds) to break the cells and release the cells' content (including soluble proteins and inclusion bodies), and then was centrifuged at 12,000 rpm and 4° C. for 20 min. The resulting pellet containing cells and IgA1 protease inclusion bodies (the majority) was suspended in 20 mL of buffer A. This sequence was repeated (3-5 times) until the supernatant became clear.

The resulting pellet containing IgA1 protease inclusion bodies was suspended in 20 mL of solubilization buffer (50 mM Tris, 150 mM NaCl, 6 M guanidine hydrochloride, pH 7.9). The suspension was sonicated on ice for 5 min, rocked at room temperature for one hour, and then centrifuged at 12,000 rpm and 4° C. for 20 min. The supernatant was collected, and the concentration of solubilized inclusion bodies therein was adjusted to 2 mg/mL by addition of the solubilization buffer.

For refolding by the dilution method, a portion (0.5 mL) of the solubilized inclusion body solution (2 mg/mL) was slowly added to 47.5 mL of refolding buffer (0.55 M guanidine hydrochloride, 0.44 M L-arginine, 55 mM Tris, 21 mM NaCl, 0.88 mM KCl, pH 7.9), and the resulting mixture was rocked at 4° C. for 1 hour. This sequence was repeated until a total of 2.5 mL of the solubilized inclusion body solution was added, where the concentration of solubilized inclusion bodies in the refolding buffer was 0.1 mg/mL. The resulting mixture was rocked at 4° C. overnight for refolding.

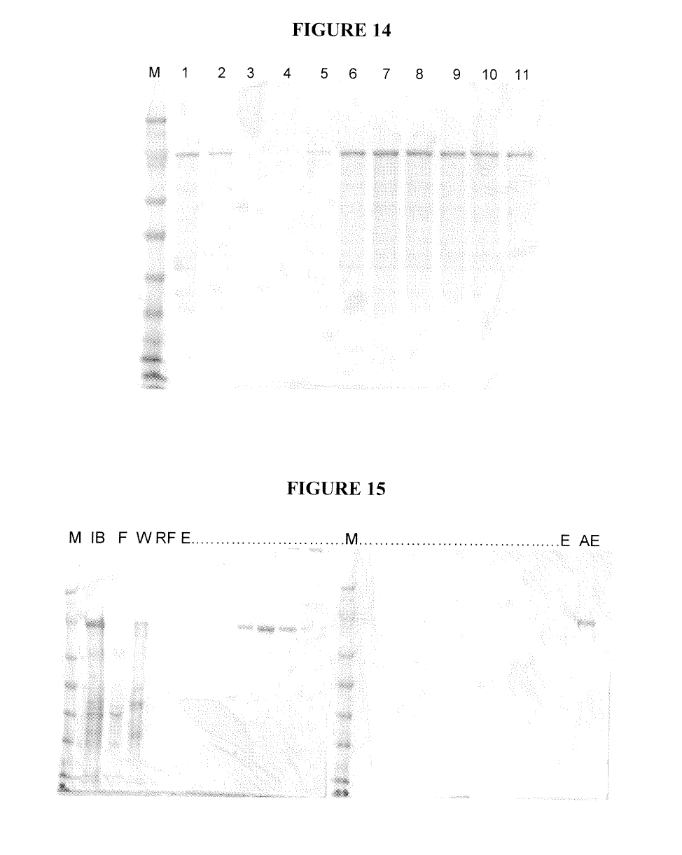

The refolded IgA1 protease in the refolding solution was dialysed with buffer A (50 mM Tris, 150 mM NaCl, pH 7.9) to change the buffer to buffer A, and then was loaded onto a Ni-NTA column (2 mL Ni-NTA bead from Qiagen, washed with 20 ml buffer A). The column was washed with 20 mL of washing buffer (50 mM Tris, 150 mM NaCl, 25 mM imidazole, pH 7.9), and then the refolded His-tagged IgA1 protease was eluted off the nickel column using an elution buffer (50 mM Tris, 150 mM NaCl, 250 mM imidazole, pH 7.9). The eluate fractions containing higher concentrations of refolded His-tagged IgA1 protease were combined (FIG. 14). The refolded His-tagged IgA1 protease underwent dialysis with PBS buffer at 4° C. overnight to change the buffer to PBS. The refolded His-tagged IgA1 protease was further purified by size-exclusion column chromatography.

### Isolation and Solubilization of Inclusion Bodies, and Refolding of Solubilized Inclusion Bodies and Purification of Refolded IgA1 Protease on a Column

Around 100 mL of bacterial culture (from fermentation (see Example 4), OD₆₀₀=189) was centrifuged at 5000 rpm for 5 min.

The resulting cell pellet was suspended in 250 mL of buffer A (50 mM Tris, 150 mM NaCl, pH 7.9) and homogenized three times to release IgA1 protease inclusion bodies from the E. coli cells, and the suspension was centrifuged at 12,000 rpm and 4° C. for 20 min. This sequence was repeated three times on the resulting pellet containing cells and inclusion bodies (the majority) to yield a pellet containing IgA1 protease inclusion bodies.

The inclusion body pellet was solubilized in 250 mL of buffer B (50 mM Tris, 150 mM NaCl, 6 M guanidine hydrochloride, pH 7.9). The resulting mixture was rocked at room temperature for one hour and then centrifuged at 12,000 rpm and 4° C. for 20 min. The concentration of solubilized IgA1 protease inclusion bodies in the supernatant was adjusted to 1 mg/mL by the addition of buffer B, and the solution was filtered.

An IMAC column was equilibrated with 100 mL of buffer B at 5 mL/min using an AKTAexplorer apparatus (GE Healthcare). One hundred mL of the filtered solution containing solubilized inclusion bodies in buffer B (1 mg/mL) was loaded onto the IMAC column at 0.5 mL/min. The column was washed with 100 mL of washing buffer C (50 mM Tris, 150 mM NaCl, 20 mM imidazole, 6 M guanidine hydrochloride, pH 7.9) at 2 mL/min. The solubilized IgA1 protease inclusion bodies were refolded by gradient wash of the column going from buffer B (50 mM Tris, 150 mM NaCl, 6 M guanidine hydrochloride, pH 7.9) or buffer D (50 mM Tris, 150 mM NaCl, 6 M urea, pH 7.9) to buffer A (50 mM Tris, 150 mM NaCl, pH 7.9, concentration of 6 M guanidine hydrochloride or 6 M urea decreasing to 0 M) at 0.5 mL/min for 2-4 hours.

The refolded His-tagged IgA1 protease was eluted off the column by gradient elution going from buffer A (50 mM Tris, 150 mM NaCl, pH 7.9) to buffer E (50 mM Tris, 150 mM NaCl, 500 mM imidazole, pH 7.9) at 2 mL/min. The column was washed with 100 mL of buffer B to elute any IgA1 protease aggregates off the column, and additional amounts of solubilized IgA1 protease inclusion bodies were loaded onto the column for refolding. Alternatively, any IgA1 protease aggregates were eluted off the column with 100 mL of buffer F (50 mM Tris, 150 mM NaCl, 6 M guanidine hydrochloride, 250 mM imidazole, pH 7.9) at 2 mL/min. Eluate fractions containing higher concentrations of refolded His-tagged IgA1 protease were combined.

Dialysis of the refolded His-tagged IgA1 protease with PBS buffer was conducted at 4° C. overnight to change the buffer to PBS. The refolded IgA1 protease was further purified by size-exclusion column chromatography.

The results of the method of refolding and purification on a column are displayed in FIG. 15. In this method, a mixture of partially purified, solubilized IgA1 protease inclusion bodies in 6 M guanidine hydrochloride (or urea) was loaded onto an IMAC column. Protein contaminants were washed away with a solution of 6 M guanidine hydrochloride (or urea) and 20 mM imidazole. The solubilized IgA1 protease inclusion bodies were refolded on the IMAC column by gradient wash using decreasing concentrations of guanidine hydrochloride (or urea). The refolded IgA1 protease was eluted off the column by gradient elution using increasing concentrations of imidazole, and was further purified by size-exclusion column chromatography. IgA1 protease aggregates were eluted off the column using a solution of 6 M guanidine hydrochloride (or urea) and 250 mM imidazole, and then dissolved in 6 M guanidine hydrochloride (or urea) for another round of refolding on the column.

### Evaluation of IgA1 Protease Refolding by Size-Exclusion Column Chromatography

A portion (0.05 mL) of the solution containing the refolded IgA1 protease was injected into a calibrated Tosoh 3000 SWXL SEC column and chromatograph (mobile phase: 2×DPBS, 0.7 mL/min for 30 min). Properly refolded IgA1 protease was read from OD₂₈₀ and a fluorescence detector according to a standard control for purified soluble, active IgA1 protease. IgA1 protease aggregates and other protein contaminants are expected to have different retention times than the properly folded IgA1 protease.

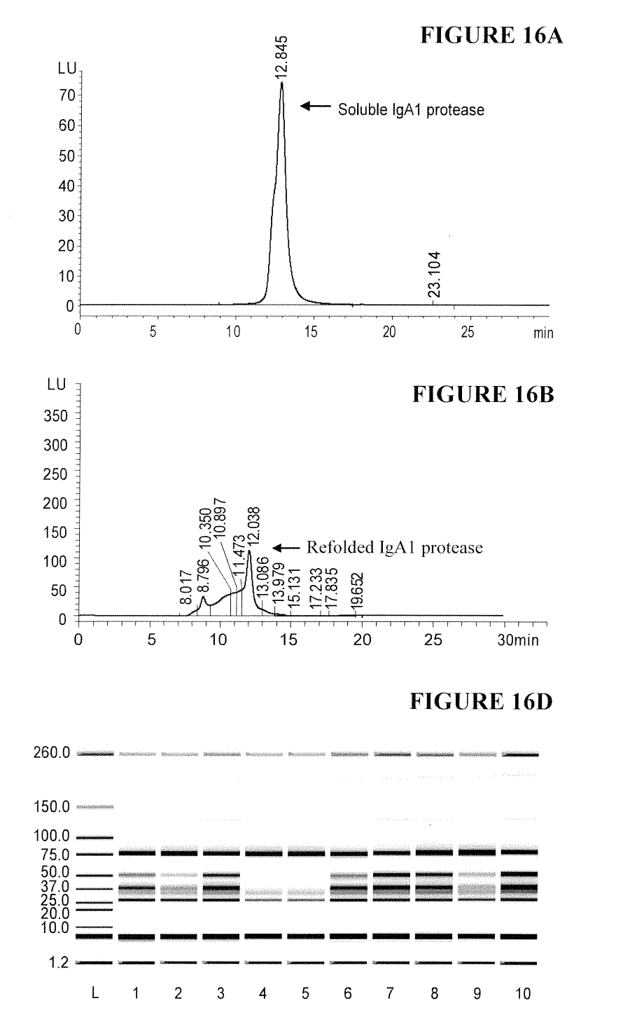

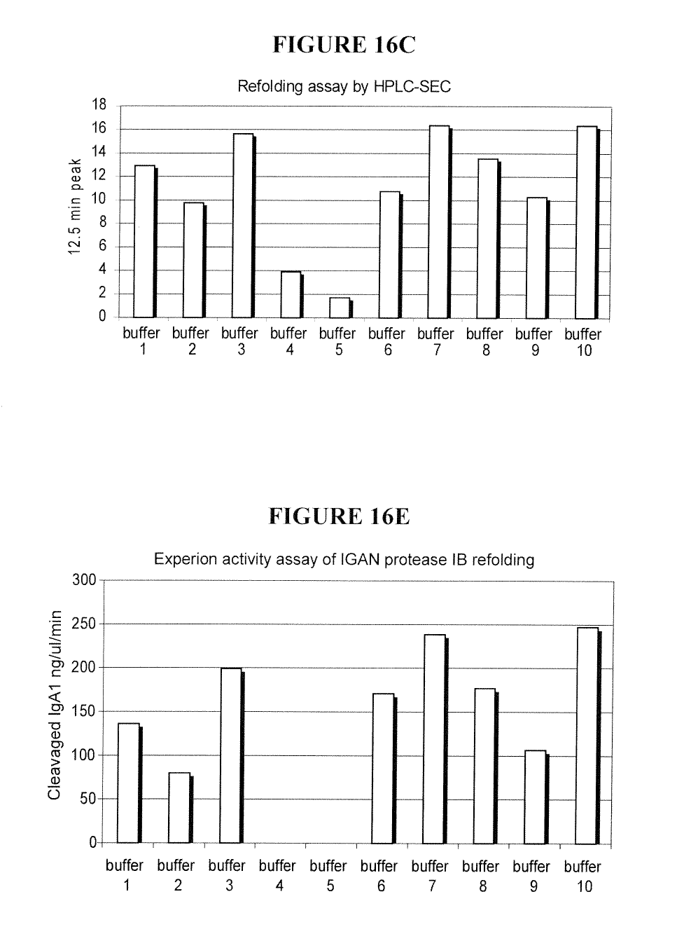

As a standard control, purified soluble, properly folded and active IgA1 protease appeared on an HPLC-SEC chromatogram as a single peak having a retention time around 12.5 minutes (FIG. 16A). Properly refolded IgA1 protease prepared by the methods described herein had a similar retention time as the IgA1 protease standard control. On the other hand, IgA1 protease aggregates and other protein contaminants had different retention times than properly folded IgA1 protease (FIG. 16B). The peak height of properly refolded IgA1 protease having a retention time around 12.5 min in HPLC-SEC varied when solubilized IgA1 protease inclusion bodies were refolded in different refolding buffers 1 to 10 (FIG. 16C), indicating that those buffers exhibited varying effectiveness in refolding IgA1 protease to its native active form. Similar results were obtained when the same samples of refolded IgA1 proteases were assayed for IgA1 cleavage activity using an Experion automated electrophoresis system (described below), where the refolded IgA1 proteases exhibiting higher peaks (or larger areas) at a retention time around 12.5 min in HPLC-SEC displayed greater IgA1 cleavage activity in the Experion assay (FIG. 16D: virtual gel of IgA1 electropherogram; FIG. 16E: IgA1 cleavage activity, in the Experion assay, of refolded IgA1 proteases formed in refolding buffers 1 to 10).

### Assay of IgA1 Protease Activity Using an Experion Automated Electrophoresis System

Human IgA1 and IgA1 protease samples were warmed up at 37° C. for 5 min prior to commencement of a reaction. Eight uL of purified human IgA1 (1600 ng/uL) was added to each PCR tube containing 1 uL of IgA1 protease sample, and the resulting mixture was incubated in a heat block at 37° C. for 0, 1, 2, 3 and 10 min. The reaction was stopped by the addition of 5 uL of sample buffer to the reaction tube followed by vortexing.

Standard samples were prepared by the addition of 5 uL of sample buffer to tubes containing 9 uL of standard human IgA1 (1600, 400, 100, 25 and 0 ng/uL). The samples and the ladder were heated at 95-100° C. for 3-5 min, and the samples were briefly centrifuged. 210 uL deionized water (0.2 micron-filtered, not autoclaved) was added to the samples and the ladder, and the resulting mixtures were vortexed. A standard curve for human IgA1 concentration was generated from the 1600, 400, 100, 25 and 0 ng/uL samples. The proteolytic activity of the IgA1 protease was measured as the decrease in human IgA1 concentration over time (ng/uL/min/ng of IgA1 protease).

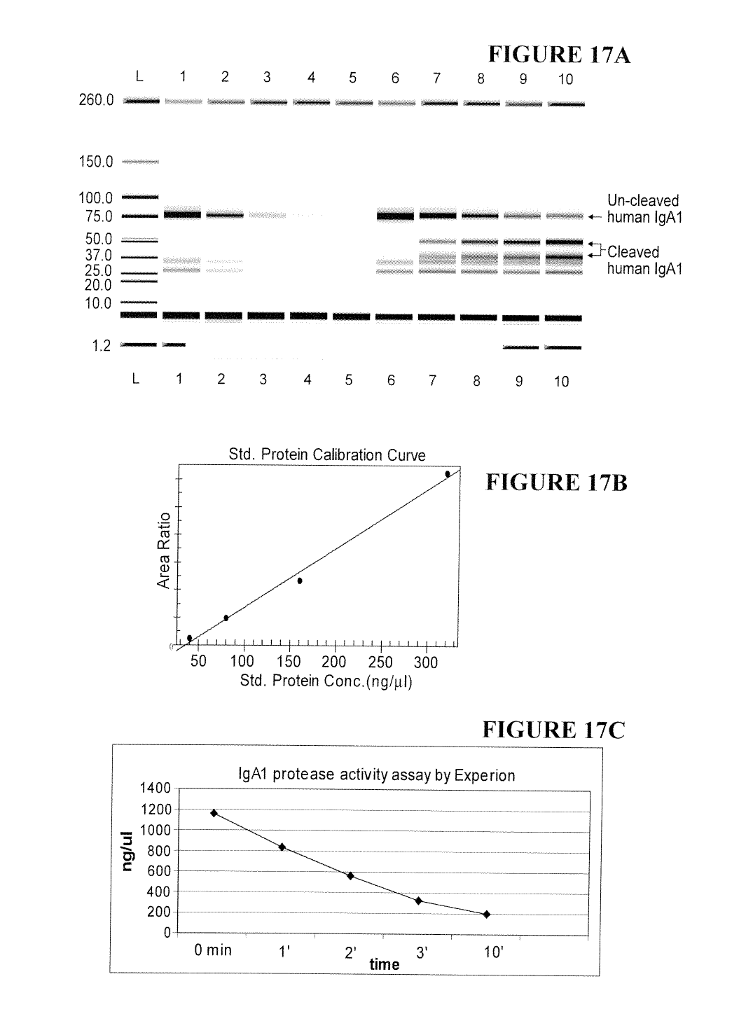

The Experion automated electrophoresis system (Bio-Rad, Hercules, Calif.) is a more convenient and quantitative way to assay IgA1 protease activity than SDS-PAGE and Western blot. Cleavage of human IgA1 was detected by Experion and displayed in a virtual gel (FIG. 17A). The amount of uncleaved human IgA1 (about 77 kDa band) decreased as it was cleaved by IgA1 protease over a period of 1, 2, 3 and 10 minutes, and correlated with an increase in the amount of cleaved IgA1 (two additional bands). A standard curve of human IgA1 was generated based on lanes 1-5 in FIG. 17A to calculate IgA1 concentration (FIG. 17B). FIG. 17C shows the human IgA1 cleavage activity of purified refolded IgA1 protease, which was calculated based on the decreasing concentration of uncleaved human IgA1 in the first minute of the assay for a more accurate assessment of proteolytic activity. The calculated human IgA1 cleavage activity of purified refolded IgA1 protease was about 50 ng IgA1/uL/min/ng protease.

### Purity and Activity of Refolded IgA1 Protease Compared to Soluble IgA1 Proteases

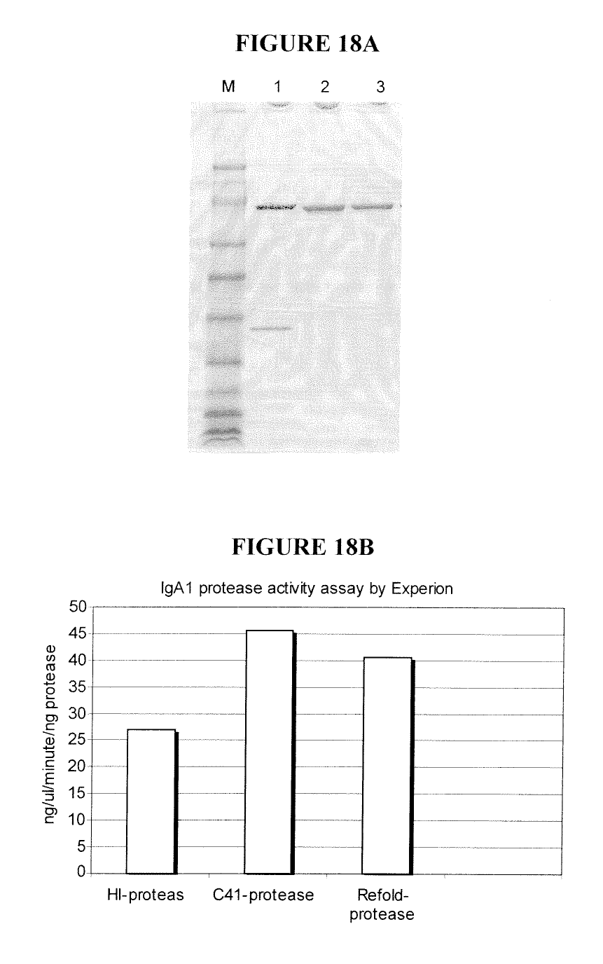

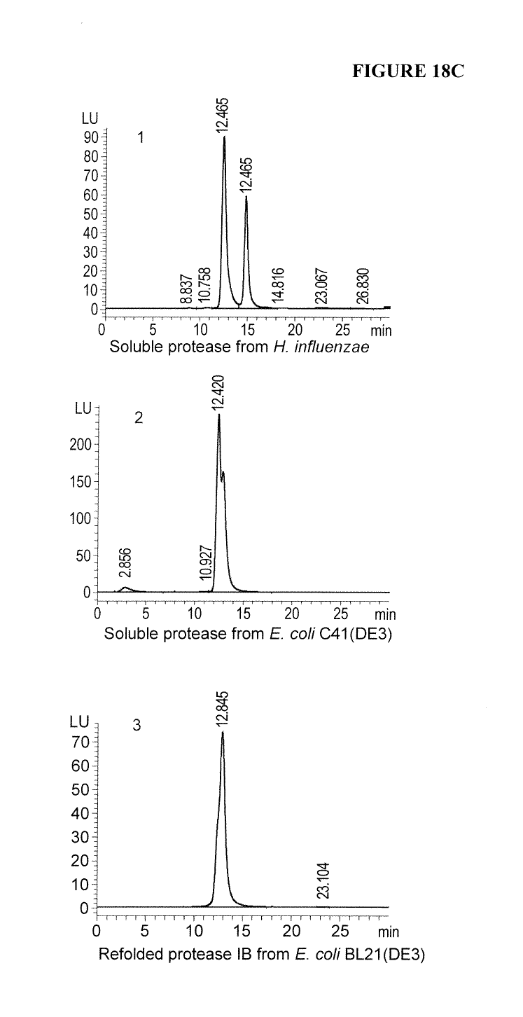

Three purified IgA1 proteases—soluble IgA1 protease directly produced from H. influenzae, soluble IgA1 protease directly produced from E. coli C41(DE3) cells, and refolded IgA1 protease prepared from inclusion bodies expressed in E. coli BL21(DE3) cells—were analyzed by SDS-PAGE (FIG. 18A), the Experion protease activity assay (FIG. 18B), and HPLC-SEC (FIG. 18C). The refolded IgA1 protease was more than 95% pure (only one peak in HPLC-SEC and only one band in SDS-PAGE) and exhibited similar human IgA1 cleavage activity as the soluble IgA1 protease directly produced from C41(DE3) cells. The soluble IgA1 protease directly produced from H. influenzae showed lower human IgA1 cleavage activity, possibly due to impurity or degradation (two peaks in HPLC-SEC and two bands in SDS-PAGE).

### Summary

E. coli BL21(DE3) cells expressed H. influenzae IgA1 protease as inclusion bodies in large amount. The inclusion bodies were readily isolated, purified, solubilized and refolded into soluble, active IgA1 protease that cleaved human IgA1. Around 1-2 g/L of soluble, active IgA1 protease was prepared from about 12 g/L of IgA1 protease inclusion bodies [BL21(DE3) fermentation having OD₆₀₀=189 and 266.6 g/L wcw (see Example 4)]. The purified refolded IgA1 protease was more than 95% pure and exhibited similar human IgA1 cleavage activity as soluble IgA1 protease directly produced from E. coli C41(DE3) cells. It is believed that the present disclosure represents the first reported preparation of active IgA1 protease from the refolding of solubilized IgA1 protease inclusion bodies.

### Example 4

### Larger-Scale Production of Active IgA1 Protease via Inclusion Bodies from BL21(DE3)

Soluble, active Haemophilus influenzae IgA1 protease containing the proteolytic protease domain and lacking the α protein and β-core domains was produced through expression of a certain amount of the IgA1 protease as insoluble inclusion bodies in E. coli BL21(DE3) cells and isolation, washing/purification, solubilization and refolding of the IgA1 protease inclusion bodies. Briefly, a certain amount of H. influenzae IgA1 protease was expressed as insoluble inclusion bodies in E. coli BL21(DE3) cells. The cells were harvested and lysed by a high-pressure homogenizer with 1×TBS to break the cell pellet and release the IgA1 protease inclusion bodies from the cells. An inclusion body pellet was collected by centrifugation.

IgA1 protease nclusion bodies were purified by either of two wash methods. In one wash method, the inclusion bodies were washed with the detergent 0.1% Triton X-100, and centrifugation was performed to separate the IgA1 protease inclusion bodies from contaminants such as DNA, soluble proteins, and lipids in the supernatant. The alternative wash method employed an automated microfiltration/crossflow filtration system, whereby the IgA1 protease inclusion bodies were suspended in 0.1% Triton X-100 and circulated through a hollow fiber filter connected to an AKTAcrossflow™ apparatus (GE Healthcare) to filter out contaminants under high pressure.

The purified IgA1 protease inclusion bodies were solubilized with either urea or guanidine hydrochloride. The solubilized inclusion bodies were then refolded to the native active form of IgA1 protease by slow dilution of the solubilized inclusion bodies in a refolding buffer containing L-arginine.

The refolded IgA1 protease was ultrafiltrated and diafiltrated (UF/DF) to remove arginine, and then purified using a nickel column (to which the histidine-tagged protease bound), an anion-exchange column, and a size-exclusion column.

### 1. Expression of IgA1 Protease

H. influenzae IgA1 protease containing the proteolytic protease domain and lacking the α protein and β-core domains was expressed in E. coli BL21(DE3) cells grown in a fermenter. A portion (about 1 mL) of a seed vial (0D₆₀₀≈10) containing BL21(DE3) cells transformed with the pET-IGAN expression construct was added to a flask containing 500 mL of LB medium (Luria broth, pH 7.0) at 37° C. The seed flask was incubated at 37° C. and agitated at 225 rpm until the culture reached a cell density between 2.0 and 4.0 OD₆₀₀. Ampicilline (50 mg/L) was added to the seed flask medium just before the whole medium was transferred to a 10 L fermenter containing about 5.8 L of a fermenter medium (8.33 g/L (NaPO₃)₆, 7.33 g/L K₂SO₄, 4.0 g/L (NH₄)₂SO₄, 1.0 mL/L polypropylene glycol Pluracol® P2000 (BASF, Mount Olive, N.J.), 25.0 g/L 70% dextrose, 1.03 g/L MgSO₄.7H₂O, 3.6 mL/L trace element solution, 50 mg/L ampicillin, 28-30% NH₄OH, pH 6.9), to an initial cell density of approximately 0.3 OD₆₀₀.

Fermentation was conducted at a temperature of 37° C., a pH of 6.9 (controlled by the automatic addition of 28-30% NH₄OH), and a dissolved oxygen concentration of 30%. The batch phase of the fermentation lasted about 6.4 hours, when glucose in the culture medium was completely consumed. The exhaustion of glucose caused a spike in pH, which initiated exponential feeding. The feed was programmed to limit the cell growth rate (μ) to 0.2/hr. After six hours of exponential feeding, the feed was fixed at 20.7 mL/min. After about 1.5 hours, induction of IgA1 protease expression was initiated by the addition of isopropyl β-D-1-thiogalactopyranoside (IPTG) to a final IPTG concentration of 1 mM. The glucose concentration was maintained at 0 g/L during the induction phase. The induction phase of fermentation continued at 35° C. or 37° C. until the cell density reached 189 OD₆₀₀.

When the cell density reached 189 OD₆₀₀, fermentation was terminated. The cells were harvested and centrifuged, and the resulting cell pellet (266.6 g/L wcw (wet cell weight)) was stored at −80° C.

### 2. Isolation of IgA1 Protease Inclusion Bodies

The crude cell pellet was thawed, and then suspended and mixed for 20 min in a buffer (50 mM Tris, 150 mM NaCl, pH 7.9), to a volume of 100 mL buffer for every 100 g wet cell pellet. The cells were homogenized by passing the cells four times through a homogenizer at 8000 psi, which lysed the cells and released IgA1 protease inclusion bodies and contaminants (e.g., DNA, lipids, non-specific proteins) from the cells, and the resulting sample was collected on ice. The sample was centrifuged at 10,000 g for 20 min, the inclusion body pellet was collected and stored at −80° C., and the contaminant-containing supernatant was discarded.

### 3. Wash/Purification of Inclusion Bodies

The wash step is designed to remove most or essentially all of the contaminants (e.g., non-specific proteins, DNA, lipids), avoid protein aggregation and increase the likelihood of successful refolding by providing purified IgA1 protease inclusion bodies for solubilization and refolding. The inclusion body pellet stored at −80° C. was thawed at room temperature and washed/purified using either of two alternative methods.

In one wash method, the inclusion body pellet was suspended in a wash buffer (50 mM Tris, 150 mM NaCl, 0.1% Triton X-100, pH 7.9). The suspension was mixed well for 15 minutes using a Turaxx impeller agitator, and then centrifuged at 4,000 g and 4° C. for 20 minutes. The contaminant-containing supernatant was decanted away, and the wash step was repeated on the retained inclusion body pellet four times. The wash method utilizing centrifugation is scalable but may be more time-consuming or labor-intensive.

Alternatively, the IgA1 protease inclusion bodies were washed/purified using an automated microfiltration/crossflow filtration system. The system contained a microfiltration hollow fiber cartridge connected to an automated, benchtop AKTAcrossflow™ apparatus (GE Healthcare). The inclusion bodies were suspended in a wash buffer (50 mM Tris, 150 mM NaCl, 0.1% Triton X-100, pH 7.9), and recirculated through a hollow fiber filter under high pressure at a permeate flow rate of 20 liters per square meter per hour (LMH). The IgA1 protease inclusion bodies were retained as the retentate, while contaminants (e.g., soluble proteins, DNA, lipids) were filtered through as the permeate under high crossflow pressure. Hollow fibers having pore sizes ranging from 0.2 μm microfiltration to a 750 kD NMWCO ultrafiltration cartridge were tested. The larger pore size cartridge (0.2 μm) removed a greater amount of contaminants. At four diavolumes of inclusion bodies recirculated through a UFP-750-E-2U 750 kD NMWCO hollow fiber cartridge, most or essentially all of the soluble protein contaminants were removed. The insoluble, washed/purified inclusion bodies were collected to be solubilized. The automated microfiltration/crossflow filtration system may be more efficient at washing/purifying inclusion bodies in larger scale, and may result in purer inclusion bodies.

### 4. Solubilization of Inclusion Bodies

The washed/purified IgA1 protease inclusion bodies were suspended in a solubilization buffer (pH 7.9, 50 mM Tris, 150 mM NaCl, and 4 M urea, 8 M urea or 6 M guanidine hydrochloride). Because urea in solution may decompose into cyanate, which in turn may react with an amino group of a polypeptide, buffers containing urea were used within 1-3 days of their preparation. The suspension in the solubilization buffer was mixed well for 15 minutes using a Turaxx agitator, and then centrifuged at 12,000 rpm and 4° C. for 20 minutes. The supernatant was collected, and the concentration of solubilized inclusion bodies therein was adjusted to 2 mg/mL by addition of the solubilization buffer.

Guanidine hydrochloride generally has stronger chaotropic properties than urea. Use of 4 M urea as the chaotropic agent resulted in 13% yield of soluble IgA1 protease and 87% yield of unsolubilized inclusion bodies. By contrast, use of 8 M urea or 6 M guanidine hydrochloride resulted in a much higher yield of soluble IgA1 protease (96% and 98%, respectively) and a much lower yield of unsolubilized inclusion bodies (4% and 2%, respectively). However, use of 6M guanidine hydrochloride resulted in a greater amount of protein aggregation in the final purified product compared to use of 8 M urea. Solubilization of the inclusion bodies with 4 M urea, 8 M urea or 6 M guanidine hydrochloride resulted in a soluble, unfolded or loosely folded, non-native IgA1 protease that was not biologically active.

### 5. Refolding of Solubilized Inclusion Bodies

A solution of the solubilized inclusion bodies (soluble IgA1 protease) in the solubilization buffer (2 mg/mL) was slowly added, over a period of about 5 hours at a rate of about 0.6 mL/min, to a refolding buffer (0.88 M L-arginine, 55 mM Tris, 21 mM NaCl, 0.88 mM KCl, pH 7.9; or 0.88 M L-arginine, 55 mM Tris, 21 mM NaCl, 0.88 mM KCl, pH 8.2; or 0.44 M L-arginine, 55 mM Tris, 21 mM NaCl, 0.88 mM KCl, pH 8.5), resulting in a final concentration of 0.1 mg/mL soluble IgA1 protease (a 20-fold dilution) for the refolding. Arginine facilitates refolding by suppressing protein aggregation, and a relatively large dilution of solubilized inclusion bodies is designed to minimize or preclude protein aggregation. The solution of solubilized inclusion bodies diluted in the refolding buffer was slowly spun at room temperature overnight for less than 24 hours. The solubilization and refolding steps resulted in about 90% yield of refolded IgA1 protease for those steps.

### 6. Purification of Refolded IgA1 Protease

The solution of refolded IgA1 protease in the refolding buffer was ultrafiltrated and diafiltrated (UF/DF) using a 50 kDa hydrostream membrane (Novasart) to remove arginine, which would interfere with the operation of a nickel column, and using an equilibration buffer of a nickel IMAC column (25 mM Na₂PO₄, 150 mM NaCl, 20 mM imidazole, pH 6.8) to buffer-exchange the protease to the equilibration buffer. UF/DF resulted in about 50% yield of refolded IgA1 protease, whose recovery may be augmented by modification of various factors—e.g., the membrane, pressure, flow rate, etc.

The refolded IgA1 protease buffer-exchanged to the equilibration buffer was loaded onto a Nickel IMAC Chelating Sepharose column (CV=42.5 mL, 8.0 cm×2.6 cm, GE Healthcare) charged with 50 mM NiSO₄ at a flow rate of 57 cm/hr, and the column was washed with the equilibration buffer at a flow rate of 113 cm/hr, during which the His-tagged protease bound to the nickel column. The refolded, His-tagged IgA1 protease was eluted off the nickel column by elution with increasing concentrations of imidazole (to 25 mM Na₂PO₄, 150 mM NaCl, 250 mM imidazole, pH 6.8) at a flow rate of 113 cm/hr. Fractions containing the main eluate product peak were combined, sterile-filtered, and diluted 10-fold with an equilibration buffer of a Q sepharose column (25 mM Tris, pH 8.0).

Chromatography with the nickel column furnished approximately 31% yield of refolded IgA1 protease of fairly high purity by HPLC-SEC. Recovery of the protease from the nickel column may be increased by optimization of various factors—e.g., elution buffer conditions, pH, total protein loading, etc.

The solution containing the main eluate product from the nickel column, diluted in the equilibration buffer, was loaded onto a Q Sepharose FF anion-exchange column (CV=14.8 mL, GE Healthcare) at a flow rate of 150 cm/hr. The column was washed with the equilibration buffer (25 mM Tris, pH 8.0) at a flow rate of 150 cm/hr. Much of the refolded IgA1 protease did not bind to the column and was collected in the flow-through. Impurities and IgA1 protease aggregates bound to the column and were eluted off the column using an elution buffer (25 mM Tris, 1 M NaCl, pH 8.0) at a flow rate of 150 cm/hr. The flow-through containing the refolded IgA1 protease was concentrated to around 20-35 mL.

Chromatography with the Q sepharose column afforded about 50% yield of refolded IgA1 protease of greater purity in the flow-through. A certain amount of refolded, unaggregated IgA1 protease came off the column in the eluate fractions, and can be combined with the refolded IgA1 protease collected in the flow-through. Recovery and purity of refolded IgA1 protease may be increased in various ways—e.g., optimization of the Q sepharose chromatography, non-use of the Q sepharose column, replacement of the Q sepharose column with other column(s) (e.g., with an anion-exchange column, such as a GigaCap Q column (Tosoh BioSciences), and/or with a hydrophobic-interaction column, such as a butyl sepharose 4 column (GE Healthcare)), etc.

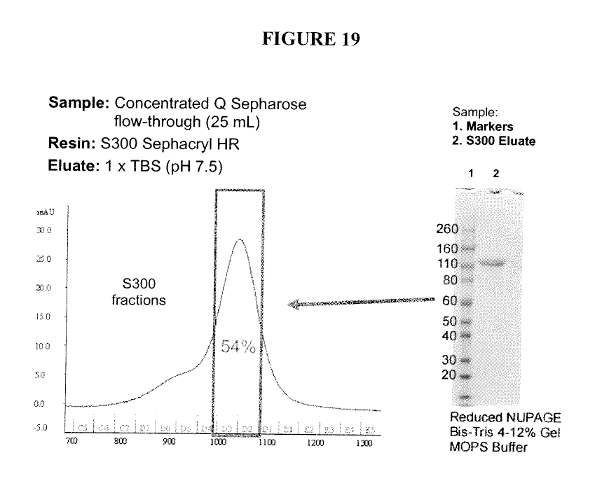

The concentrated flow-through solution from the Q sepharose column was loaded onto an S300 Sephacryl HR size-exclusion column (CV=1.8765×95 cm, GE Healthcare), which purifies proteins by their different sizes. The refolded IgA1 protease was eluted off the column using a mobile phase of 1×TBS (Tris-buffered saline—25 mM Tris, 150 mM NaCl, pH 7.5) at a flow rate of 30 cm/hr, and fractions containing the main eluate product peak were collected. Chromatography with the S300 column indicated the presence of IgA1 protease aggregates, which were separated from refolded, unaggregated IgA1 protease by collection of appropriate eluate fractions (FIG. 19). Solubilization of IgA1 protease inclusion bodies with 6 M guanidine hydrochloride resulted in a greater amount of IgA1 protease aggregates than solubilization with 8 M urea.

Chromatography with the S300 column provided about 54% yield of refolded, unaggregated IgA1 protease (e.g., 7.2 mg recovered from 13.4 mg of loaded material) of high purity (FIG. 19), and in a formulation buffer (TBS), for biological testing. The purified refolded IgA1 protease can be utilized in in vitro assays (e.g., assessing cleavage of human IgA1) and in vivo studies (e.g., animal models of IgA nephropathy).

### Automation of Washing, Solubilization and Refolding

The AKTAcrossflow™ apparatus can be employed to automate the wash, solubilization and refolding steps. The AKTAcrossflow™ apparatus takes the isolated inclusion body pellet and washes the IgA1 protease inclusion bodies through a hollow fiber cartridge connected to the apparatus. After washing/purification of the inclusion bodies, the apparatus adds a buffer containing urea or guanidine hydrochloride to a holding vessel, where the inclusion bodies are solubilized. Then the AKTAcrossflow™ apparatus slowly adds the mixture comprising the solubilized inclusion bodies to a container comprising a refolding buffer, where the solubilized inclusion bodies are allowed to refold into soluble, active IgA1 protease over a period of time. The apparatus then ultrafiltrates and diafiltrates (UF/DF) the refolded IgA1 protease through a membrane filter to remove arginine (if the refolding buffer contains arginine) and prepare the refolded protein for purification using any of the methods and techniques described herein.

Unlike previous attempts to express IgA proteases recombinantly in E. coli cells, the present methods allow for direct production of significant amounts of soluble, active IgA (e.g., IgA1) protease without having to extract the protease from inclusion bodies and refold the protease. The examples herein demonstrate protein yields of about 20-40 mg/L of soluble and active IgA (e.g., IgA1) protease (e.g., expression of only the IgA1 protease proteolytic domain in the C41(DE3) cell line at 20° C. and 0.4 mM IPTG). Not intending to be bound by theory, a possible reason why the present methods can directly produce significant amounts of soluble and active IgA proteases is that the host cells (e.g., E. coli) express only the proteolytic protease domain and not the full-length precursor protein, and thus the expressed polypeptide does not need to be cleaved into the mature protease, unlike previous recombinant expressions in H. influenzae and other bacteria. It is believed that the present disclosure represents the first disclosure of expression of only the IgA protease proteolytic domain, and neither the α protein domain nor the β-core domain, for recombinant production of soluble and active IgA proteases (e.g., IgA1 protease).

Further, the methods described herein can produce at least about 10-20 g/L of IgA (e.g., IgA1) protease inclusion bodies that can be solubilized and refolded to the active form of IgA protease (e.g., expression of only the IgA1 protease proteolytic domain in the BL21(DE3) cell line at 37° C. and 1 mM IPTG). Through solubilization, refolding and purification, at least about 1-2 g/L of soluble and active IgA (e.g., IgA1) protease can be prepared from at least about 10-20 g/L of IgA protease inclusion bodies. The total yield of soluble, active IgA protease produced by the methods described herein, whether by direct production or indirect production via inclusion bodies, or both, is at least about 100-fold greater than that achieved by previous methods for recombinant production of bacterial IgA proteases (e.g., about 0.3 mg/L of secreted IgA1 protease produced by Haemophilus influenzae cells grown in heme-containing media from bovine serum).

The methods described herein produce increased yields of IgA proteases (e.g., IgA1 proteases) and thereby allow for production of IgA proteases in amounts useful for administration of the IgA proteases (e.g., IgA1 proteases) to treat IgA deposition disorders, such as IgA nephropathy, certain liver and kidney diseases, and other disorders described herein.

It is understood that every embodiment of the present disclosure may optionally be combined with any one or more of the other embodiments described herein. Every patent literature, and every non-patent literature, cited herein are incorporated herein by reference in their entirety to the extent that they are not inconsistent with the present disclosure.

Numerous modifications and variations to the present disclosure, as set forth in the embodiments and illustrative examples described herein, will be apparent to persons of ordinary skill in the art. All such modifications and variations are intended to be within the scope of the present disclosure and the appended claims.

What is claimed is:

1. A method for producing a serine-type IgA protease from a host cell, comprising growing a host cell comprising a vector, the vector comprising a polynucleotide encoding an IgA protease polypeptide that comprises an IgA protease proteolytic domain and lacks at least about 50% of an α protein domain and at least about 50% of a β-core domain, under conditions that result in expression of the IgA protease polypeptide as inclusion bodies, or as a soluble polypeptide that exhibits IgA protease activity, or a combination thereof.

2. The method of claim 1, further comprising:
isolating the inclusion bodies;
solubilizing the isolated inclusion bodies; and
refolding the solubilized inclusion bodies into soluble, active IgA protease.

3. The method of claim 2, wherein the solubilizing comprises using a chaotropic agent selected from the group consisting of urea, guanidine hydrochloride (guanidinium chloride), lithium perchlorate, formic acid, acetic acid, trichloroacetic acid, sulfosalicylic acid, sarkosyl, and combinations thereof.

4. The method of claim 3, wherein the chaotropic agent is at a concentration from about 4 M to about 10 M.

5. The method of claim 2, wherein the solubilized inclusion bodies are refolded in a refolding buffer that:
(a) comprises Tris and NaCl, and has a pH from about 7 to about 9.5; or
(b) comprises CHES and NaCl, and has a pH from about 8 to about 10; or
(c) comprises MES and NaCl, and has a pH from about 5 to about 7; or
(d) comprises phosphate-buffered saline (PBS), and has a pH from about 6 to about 8.

6. The method of claim 5, wherein the refolding buffer further comprises arginine.

7. The method of claim 6, wherein the arginine is at a concentration from about 0.05 M to about 1.5 M.

8. The method of claim 2, wherein the solubilized inclusion bodies are refolded at a temperature from about 4° C. to about 30° C.

9. The method of claim 2, wherein the solubilized inclusion bodies are at a concentration from about 0.01 mg/mL to about 1 mg/mL during refolding.

10. The method of claim 2, wherein the isolated inclusion bodies are solubilized using urea, and the solubilized inclusion bodies are refolded in a refolding buffer that comprises Tris, lacks added arginine, and has a pH from about 7.5 to about 9.5.

11. The method of claim 10, wherein the refolding buffer further comprises NaCl or glycerol, or a combination thereof.

12. The method of claim 10, wherein the isolated inclusion bodies are solubilized using about 7-9 M urea, and the solubilized inclusion bodies are refolded in a refolding buffer that lacks added arginine, has a pH from about 7.8 to about 9, and comprises (a) about 30-70 mM Tris, or (b) about 30-70 mM Tris and about 50-250 mM NaCl, or (c) about 30-70 mM Tris and about 5-15% glycerol.

13. The method of claim 2, further comprising washing the isolated inclusion bodies prior to solubilizing the isolated inclusion bodies.

14. The method of claim 13, wherein the washing comprises centrifuging the isolated inclusion bodies or microfiltering the isolated inclusion bodies through a hollow fiber with cross flow filtration.

15. The method of claim 2, further comprising purifying the refolded IgA protease.

16. The method of claim 15, wherein the purifying comprises using a nickel column, an anion-exchange column, a cation-exchange column, a hydrophobic-interaction column, or a size-exclusion column, or a combination thereof.

17. The method of claim 2, which results in at least about 1-2 g/L of soluble, active IgA protease from at least about 10-20 g/L of IgA protease inclusion bodies.

18. The method of claim 1, further comprising isolating the soluble, active IgA protease polypeptide.

19. The method of claim 18, which results in at least about 20-40 mg/L of soluble, active IgA protease polypeptide.

20. The method of claim 1, wherein the growing of the host cell comprising the vector results in at least about a 10-fold, 50-fold or 100-fold higher production of soluble, active IgA protease, by direct production or indirect production via inclusion bodies, or a combination thereof, compared to culturing under the same conditions a host cell comprising a vector that encodes the entirety of the α protein domain and the β-core domain.

21. The method of claim 1, wherein the IgA protease is selected from the group consisting of Haemophilus influenza IgA proteases, Neisseria gonorrhoeae IgA proteases, and Neisseria meningitidis IgA proteases.

22. The method of claim 1, wherein the IgA protease is an IgA1 protease.

23. The method of claim 1, wherein the IgA protease is at least about 60% identical to SEQ ID NO: 1, 2, 3, 4, 5, 6, 7, 8, 9, 10, 11, 12, 22 or 23.

24. The method of claim 1, wherein the host cell is selected from the group consisting of E. coli, Bacillus, Streptomyces, and Salmonella strains and cell lines.

25. The method of claim 24, wherein the E. coli strains and cell lines are selected from the group consisting of BL21(DE3), BL21(DE3)pLysS, BL21(DE3)pGro7, ArcticExpress, ArcticExpress(DE3), C41(DE3), C43(DE3), Origami B, Origami B(DE3), Origami B(DE3)pLysS, KRX, and Tuner(DE3).

26. The method of claim 1, wherein the host cell is grown for a time period at a temperature from about 10° C. to about 40° C.

27. The method of claim 1, wherein expression of the polynucleotide is enhanced using an isopropyl β-D-1-thiogalactopyranoside (IPTG)-inducible vector.

28. The method of claim 27, wherein the host cell is grown for a time period at a temperature from about 10° C. to about 40° C. when cultured with IPTG.

29. The method of claim 27, wherein the host cell is cultured with IPTG at a concentration from about 0.2 mM to about 2 mM.

30. The method of claim 1, wherein the vector is a plasmid selected from the group consisting of pET21a, pColdIV, pJexpress401, pHT01, pHT43, and pIBEX.

31. The method of claim 30, wherein the plasmid comprises a promoter selected from the group consisting of a T7 promoter, a T5 promoter, a cold shock promoter, and a pTAC promoter.

32. The method of claim 1, wherein the polynucleotide further encodes a signal peptide.

33. A host cell comprising a vector, the vector comprising a polynucleotide encoding a serine-type IgA protease polypeptide that comprises an IgA protease proteolytic domain and lacks at least about 50% of an α protein domain and at least about 50% of a β-core domain, wherein the IgA protease polypeptide is expressed from the host cell as inclusion bodies, or as a soluble polypeptide that exhibits IgA protease activity, or a combination thereof.

34. The host cell of claim 33, wherein the IgA protease is selected from the group consisting of Haemophilus influenza IgA proteases, Neisseria gonorrhoeae IgA proteases, and Neisseria meningitidis IgA proteases.

35. The host cell of claim 33, wherein the IgA protease is an IgA1 protease.

36. The host cell of claim 33, wherein the IgA protease is at least about 60% identical to SEQ ID NO: 1, 2, 3, 4, 5, 6, 7, 8, 9, 10, 11, 12, 22 or 23.

37. The host cell of claim 33, wherein the host cell is selected from the group consisting of E. coli, Bacillus, Streptomyces, and Salmonella strains and cell lines.

38. The host cell of claim 37, wherein the E. coli strains and cell lines are selected from the group consisting of BL21(DE3), BL21(DE3)pLysS, BL21(DE3)pGro7, ArcticExpress, ArcticExpress(DE3), C41(DE3), C43(DE3), Origami B, Origami B(DE3), Origami B(DE3)pLysS, KRX, and Tuner(DE3).

39. The host cell of claim 33, wherein the vector is a plasmid selected from the group consisting of pET21a, pColdIV, pJexpress401, pHT01, pHT43, and pIBEX.

40. A composition comprising at least about 50 grams or 75 grams wet weight of the host cell of claim 33.


SEQUENCE LISTING (24 sequences)

SEQ ID NO: 1 - protein, 1694 aa, Haemophilus influenzae
MLNKKFKLNFIALTVAYALTPYTEAALVRDDVDYQIFRDFAENKGRFSVGATNVEVRDKNNHSLGNVLPNGIPMIDFSVVDVDKRIATLINPQYVVGVKHVSNGVSELHFGNLNGNMNNGNAKSHRDVSSEENRYFSVEKNEYPTKLNGKAVTTEDQTQKRREDYYMPRLDKFVTEVAPIEASTASSDAGTYNDQNKYPAFVRLGSGSQFIYKKGDNYSLILNNHEVGGNNLKLVGDAYTYGIAGTPYKVNHENNGLIGFGNSKEEHSDPKGILSQDPLTNYAVLGDSGSPLFVYDREKGKWLFLGSYDFWAGYNKKSWQEWNIYKPEFAKTVLDKDTAGSLTGSNTQYNWNPTGKTSVISNGSESLNVDLFDSSQDTDSKKNNHGKSVTLRGSGTLTLNNNIDQGAGGLFFEGDYEVKGTSDSTTWKGAGVSVADGKTVTWKVHNPKSDRLAKIGKGTLIVEGKGENKGSLKVGDGTVILKQQADANNKVKAFSQVGIVSGRSTVVLNDDKQVDPNSIYFGFRGGRLDANGNNLTFEHIRNIDDGARLVNHNTSKTSTVTITGESLITDPNTITPYNIDAPDEDNPYAFRRIKDGGQLYLNLENYTYYALRKGASTRSELPKNSGESNENWLYMGKTSDEAKRNVMNHINNERMNGFNGYFGEEEGKNNGNLNVTFKGKSEQNRFLLTGGTNLNGDLKVEKGTLFLSGRPTPHARDIAGISSTKKDQHFAENNEVVVEDDWINRNFKATNINVTNNATLYSGRNVANITSNITASDNAKVHIGYKAGDTVCVRSDYTGYVTCTTDKLSDKALNSFNATNVSGNVNLSGNANFVLGKANLFGTISGTGNSQVRLTENSHWHLTGDSNVNQLNLDKGHIHLNAQNDANKVTTYNTLTVNSLSGNGSFYYLTDLSNKQGDKVVVTKSATGNFTLQVADKTGEPTKNELTLFDASNATRNNLNVSLVGNTVDLGAWKYKLRNVNGRYDLYNPEVEKRNQTVDTTNITTPNNIQADVPSVPSNNEEIARVETPVPPPAPATPSETTETVAENSKQESKTVEKNEQDATETTAQNGEVAEEAKPSVKANTQTNEVAQSGSETEETQTTEIKETAKVEKEEKAKVEKDEIQEAPQMASETSPKQAKPAPKEVSTDTKVEETQVQAQPQTQSTTVAAAEATSPNSKPAEETQPSEKTNAEPVTPVVSKNQTENTTDQPTEREKTAKVETEKTQEPPQVASQASPKQEQSETVQPQAVLESENVPTVNNAEEVQAQLQTQTSATVSTKQPAPENSINTGSATAITETAEKSDKPQTETAASTEDASQHKANTVADNSVANNSESSDPKSRRRRSISQPQETSAEETTAASTDETTIADNSKRSKPNRRSRRSVRSEPTVTNGSDRSTVALRDLTSTNTNAVISDAMAKAQFVALNVGKAVSQHISQLEMNNEGQYNVWVSNTSMNENYSSSQYRRFSSKSTQTQLGWDQTISNNVQLGGVFTYVRNSNNFDKASSKNTLAQVNFYSKYYADNHWYLGIDLGYGKFQSNLKTNHNAKFARHTAQFGLTAGKAFNLGNFGITPIVGVRYSYLSNANFALAKDRIKVNPISVKTAFAQVDLSYTYHLGEFSVTPILSARYDTNQGSGKINVNQYDFAYNVENQQQYNAGLKLKYHNVKLSLIGGLTKAKQAEKQKTAELKLSFSF

SEQ ID NO: 2 - protein, 1887 aa, Haemophilus influenzae
MLNKKFKLSLITLSVIYALTPYTEAALVRDDVDYQIFRDFAENKGKFFVGATDLSVKNKQGQNIGNALSNVPMIDFSVADVNKRIATVVDPQYAVSVKHAKAEVHTFYYGQYNGHNDVADKENEYRVVEQNNYEPHKAWGASNLGRLEDYNMARFNKFVTEVAPIAPTDAGGGLDTYKDKNRFSSFVRVGAGRQLVYEKGAYHQEGNEKGYDLRDLSQAYRYAIAGTPYKDINIDQTMNTEGLIGFGHHNTHYSAEELKQALSQDALTNYGVLGDSGSPLFAFDKQKNQWVFLGTYDYWAGYGKKSWQEWNIYKKEFADKIKQHDNAGTIKGNGEHHWKTTGTNSHIGSTAVRLANNERDANNGQNVTFEDNGTLVLDQNINQGAGGLFFKGDYTVKGANSDITWLGAGIDVADGKKVVWQVKNPQGDKLAKIGKGALEINGTGVNQGELKVGDGTVILNQKADSNQKVQAFSQVGIVSGRGTLVLNSPDQINPNNLYFGFRGGRLDANGNDLTFEHIRNVDEGARVVNHNTSNASTITLTGKSLITDPKGLSIHYIQNNDYDDDGYYGYYYRPRKPIPQGKDLYFKNYRYYALKPGGSVNSPMPENGVAENNDWVFMGYTEEKAKKNVMNHKNNQRISGFSGFFGEENGKGHNGALNLNFNGKSAQNRFLLTGGTNLNGKISVTQGNVLLSGRPTPHARDFVNKSSAYKDAHFSKNNEVVFEDDWINRTFKAAEITVNQSASLSSGRNVSNITANITATDNAKVNLGYKNGDEVCVRSDYTGYVTCTKDNLSDKALNSFDATQINGNVNLSQNAALTLGKAALWGQIQGQGNSRVSLNQHSKWHLTGDSQVQNLSLEDSHIHLNNASDAQSANKYHTLKINHLSGNGHFHYLTHLAKNLGDKVVVKESASGHYQLHVQDKTGEPNQEGLDLFDASSVQDRSRLSVSLANHHVDLGALRYTIKTENGITRLYNPYAENRRRVKPVPSPATNTASQAQKATQTDGAQIAKPQNIVIAPPSPQANQAEEAKRQQAEAEKVARRKAEEAKRQAAELLAKQKAEAEAQALAARRQAEAERKARELAEREKAEAERKAAELAKQKAEQAKAQPKRRRRRAAPQNNVAIAQAQEARRQQAEAERVARLKAEEAKRQSEMLARQKSEEERKARELAEREKAEAEKVARRKAEEAKRQAAELLAKQKAEAEAQALAARRQAEAERKARELAEREKAEAERKAAELAKQKAEQAKAQPKRRRRRAAPQNNVAIAQAQEARRQQAEAERVARLKAEEAKRQSEMLARQKSEEERKARELAEREKAEAEKVARRKAEEAKRQAAELLAKQKAEAEAQALAARRQAEAERKARELAEREKAEAERKAAELAKQKAEQAKAQPKRRRRRAAPQNNVAIAQAQEARRQQAEAERVARLKAEEAKRQSEMLARQKSEEERKARELAEREKAEAERKAEELAKQKAEEASHQAKVQPKRRRRRAILPRPPAPVFSLDDYDAKDNSESSIGNLARVTPRMKRELIDDFEEIPLSALEEAETTTNITDNIGKDIQEILDDEFENTDIEPLIDSLGQVVRLQPRTLSPMENMSQAQAISKNTNTALSDAMVSSQFILLDTGSSLVQQITQTELSANKENNVWVSNTTYDRHYSSTQYRQFSAKRSQIQIGIDHYLSKNTQVGTVLSYVRNSNVFDQASGKNTFVQANTYGKYYFDYGWYISGDIGVGQLRSQLQTQQKAKFNRIATQAGIMIGNRIDINRFEILPSIGVRYSYLSSIDYKLGSDSLKVDSISIKTALAKLDLAYQFNIGEFALKPILSMAYVINSGEGIVNIGGQNYRYKSDNQQQYSAGMALNYRSLTFNINGGAIKGRQLSNQKFLQIKMQVSF

SEQ ID NO: 3 - protein, 1794 aa, Haemophilus influenzae
MLNKKFKLNFIALTVAYALTPYTEAALVRNDVDYQIFRDFAENKGKFSVGATNVEVRDNKNNNLGSALPKDIPMIDFSAVDVDKRIATLVNPQYVVGVKHVGNGVGELHFGNLNGNWNPKFGNSIQHRDVSWEENRYYTVEKNNFSSELNGKTQNNEKDKQYTSNKKDVPSELYGQALVKEQQNQKRREDYYMPRLDKFVTEVAPIEASTTSSDAGTYNDQNKYPAFVRLGSGSQFIYKKGSHYELILEEKNEKKEIIHRWDVGGDNLKLVGNAYTYGIAGTPYKVNHTDDGLIGFGDSTEDHNDPKEILSRKPLTNYAVLGDSGSPLFVYDKSKEKWLFLGAYDFWGGYKKKSWQEWNIYKPQFAENILKKDSAGLLKGNTQYNWTSKGNTSLISGTSESLSVDLVDNKNLNHGKNVTFEGSGNLTLNNNIDQGAGGLFFEGDYEVKGTSENTTWKGAGISVAEGKTVKWKVHNPQFDRLAKIGKGKLIVEGRGDNKGSLKVGDGTVVLKQQTTTGQHAFASVGIVSGRSTVVLNDDNQVDPNSIYFGFRGGRLDANGNNLTFEHIRNIDDGARLVNHNMTNASNITITGAGLITNPSQVTIYTPAITADDDNYYYVPSIPRGKDLYFSNTCYKYYALKQGGSPTAEMPCYSSEKSDANWEFMGDNQNDAQKKAMVYINNRRMNGFNGYFGEEATKADQNGKLNVTFSGKSDQNRFLLTGGTNLNGELKVEKGTLFLSGRPTPHARDIANISSTEKDKHFAENNEVVVEDDWINRTFKATNINVTNNATLYSGRNVESITSNITASNKAKVHIGYKAGDTVCVRSDYTGYVTCHNDTLSTKALNSFNPTNLRGNVNLTESANFTLGKANLFGTINSTENSQVNLKENSHWYLTGNSDVHQLDLANGHIHLNNVSDATKETKYHTLNISNLSGNGSFYYWVDFTKNQGDKVVVTKSAKGTFTLQVANKTGEPNHNELTLFDASNATERSGLNVSLANGKVDRGAWSYTLKENSGRYYLHNPEVERRNQTVDTPSIATANNMQADVPSVSNNHEETARVEAPIPLPAPPAPATGSAMANEQPETRPAETVQPTMEDTNTTHPSGSEPQADTTQADDPNSESVPSETIEKVAENSPQESETVAKNEQKATETTAQNDEVAKEAKPTVEANTQTNELAQNGSETEETQEAETARQSEINSTEETVVEDDPTISEPKSRPRRSISSSSNNINLAGTEDTAKVETEKTQEAPQVAFQASPKQEEPEMAKQQEQPKTVQSQAQPETTTQQAEPARENVSTVNNVKEAQPQAKPTTVAAKETTASNSEQKETAQPVANPKTAENKAENPQSTETTDENIHQPEAHTAVASTEVVTPENATTPIKPVENKTTEAEQPVTETTTVSTENPVVKNPENTTPATTQSTVNSEAVQSETATTEAVVSQSKVTSAEETTVASTQETTVDNSGSTPQPRSRRTRRSAQNSYEPVELHTENAENPQSGNDVATQLVLRDLTSTNTNAVISDAMAKAQFVALNVGKAVSQHISQLEMNNEGQYNVWVSNTSMKENYSSSQYRHFSSKSAQTQLGWDQTISSNVQLGGVFTYVRNSNNFDKASSKNTLAQANLYSKYYMDNHWYLAVDLGYGNFQSNLQTNHNAKFARHTAQFGLTAGKAFNLGNFAVKPTVGVRYSYLSNANFALAKDRIKVNPISVKTAFAQVDLSYTYHLGEFSITPILSARYDANQGSGKINVDRYDFAYNVENQQQYNAGLKLKYHNVKLSLIGGLTKAKQAEKQKTAEVKLSFSF

SEQ ID NO: 4 - protein, 1794 aa, Haemophilus influenzae; identical to SEQ ID NO: 3

SEQ ID NO: 5 - protein, 1694 aa, Haemophilus influenzae
MLNKKFKLNFIALTVAYALTPYTEAALVRDDVDYQIFRDFAENKGRFSVGATNVEVRDKNNHSLGNVLPNGIPMIDFSVVDVDKRIATLINPQYVVGVKHVSNGVSELHFGNLNGNMNNGNAKSHRDVSSEENRYFSVEKNEYPTKLNGKAVTTEDQTQKRREDYYMPRLDKFVTEVAPIEASTASSDAGTYNDQNKYPAFVRLGSGSQFIYKKGDNYSLILNNHEVGGNNLKLVGDAYTYGIAGTPYKVNHGVNGLIGFGNSKEEHSDPKAILSQDPLTNYAVLGDSGSPLFVYDREKGKWLFLGSYDFWAGYNKKSWQEWNIYKPEFAKTVLDKDTAGSLTGSNTQYNWNPTGKTSVISNGSESLNVDLFDSSQDTDSKKNNHGKSVTLRGSGTLTLNNNIDQGAGGLFFEGDYEVKGTSDSTTWKGAGVSVADGKTVTWKVHNPKSDRLAKIGKGTLIVEEKGENKGSLKVGDGTVILKQQADANNKVKAFSQVGIVSGRSTVVLNDDKQVDPNSIYFGFRGGRLDANGNNLTFEHIRNIDDGARLVNHNTSKTSTVTITGESLITDPNTITPYNIDAPDEDNPYAFRRIKDGGQLYLNLENYTYYALRKGASTRSELPKNSGESNENWLYMGKTSDEAKRNVMNHINNERMNGFNGYFGEEEGKNNGNLNVTFKGKSEQNRFLLTGGTNLNGDLKVEKGTLFLSGRPTPHARDIAGISSTKKDQHFAENNEVVVEDDWINRNFKATNINVTNNATLYSGRNVANITSNITASDNAKVHIGYKAGDTVCVRSDYTGYVTCTTDKLSDKALNSFNATNVSGNVNLSGNANFVLGKANLFGTISGTGNSQVRLTENSHWHLTGDTNVNQLNLDKGHIHLNAQNDANKVTTYNTLTVNSLSGNGSFYYLTDLSNKQGDKVVVTKSATGNFTLQVADKTGEPTKNELTLFDASNATRNNLNVSLVGNTVDLGAWKYKLRNVNGRYDLYNPEVEKRNQTVDTTNITTPNNIQADVPSVPSNNEEIARVETPVPPPAPDTPSETTETVAENSKQESKTVEKNEQDATETTAQNGEVGEEAKPSVKANTQTNEVAQSGSETEETQTTEIKETAKVEKEEKAKVEKDEIQEAPQMASETSPKQAKPAPKEVSTDTKVEETQVQAQPQTQSTTVAAAEATSPNSKPAEETQPSEKTNAEPVTPVVSKNQTENTTDQPTEREKTAKVETEKTQEPPQVASQASPKQEQSETVQPQAVLESENVPTVNNAEEVQAQLQTQTSATVSTKQPAPENSINTGSATAITETAEKSDKPQTETAASTEDASQHKANTVADNSVANNSESSDPKSRRRRSISQPQETSAEETTAASTDETTIADNSKRSKPNRRSRRSVRSEPTVTNGSDRSTVALRDLTSTNTNAVISDAMAKGQFVALNVGKAVSQHISQLEMNNEGQYNVWVSNTSMNENYSSSQYRRFSSKSTQTQLGWDQTISNNVQLGGVFTYVRNSNNFDKASSKNTLAQVNFYSKYYADNHWYLGIDLGYGKFQSNLKTNTNAKFARHTAQFGLTAGKAFNLGNFGITPIVGVRYSYLSNANFALAKDRIKVNPISVKTAFAQVDLSYTYHLGEFSVTPILSARYDTNQGSGKINVNQYDFAYNVENQQQYNAGLKLKYHNVKLSLIGGLTKAKQAEKQKTAELKLSFSF

SEQ ID NO: 6 - protein, 1852 aa, Haemophilus influenzae
MLNKKFKLNFIALTVAYALTPYTEAALVRNDVDYQIFRDFAENKGKFSVGATNVEVRDKNNHSLGNVLPNGIPMIDFSVVDVDKRIATLVNPQYVVGVKHVGNGVSELHFGNLNGNMNNGNAKAHRDVSSEENRYFSVEKNDFPSTVNPQNEQKRREDYYMPRLDKFVTEVAPIEPSTDSSKKGTYNNKEKYPAFVRLGSGTQFIYEKNGSYDDRWVISGREQPVHYSNLKLVGKAYTYGIAGTPYTVNHVTDGLVGFGDSTQKHIDPKEILSQDPLTNYAVLGDSGSPLFVYDKEKGKWLFLGSYDYWAGYDKKSWQEWNIYKPEFATEVLNKYNAGSLTGANTQYNWNSTDNTSIISSDSKSLNVDLFDSSQDTDSKKNNHGKSVTLRGSGTLILNSNINQGAGGLFFEGDYEVKGTSENTTWKGAGISVAEGKTVKWKVHNPQSDRLAKIGEGTLVVQGKGENKGQLKVGDGKVILKQEADSSGKVKAFSMLGIVSGRSTVVLNDDKQVDPNSIYFGFRGGRLDANGNNLTFEHIRNIDDGARVVNHNMTNTSNITITGTGLITNPSQVTLGYIQARDEDNPYAPRRIKDGYQLYFDEENRNYYTLRKGAKFNSQLPYNDNESNETWLYMGKNSDEAKKKTMEYINNSRMNGFNGYFGEEETKATQNGKLNVTFNGKSEQNRFLLTGGTNLNGDLNVQQGTLFLSGRPTPHARDIAGISSTKKDKHFSENNEVVVEDDWINRNFKATNINVTNNATLYSGRNVESITSNITASSTAQVHIGYKAGDTVCVRSDYTGYVTCHNGTLSTKALNSFNATNVSGNVNLSDNANFVLGKANLFGTIQSTGTSQVNLKENSHWHLTGNSDVHQLDLANGHIHLNSADNSNNVTKYNTLTVNSLSGNGSFYYWVDFTNNKSDKVVVTQSAKGNFTLQVADKTGEPNHNELTLFDASNATRSNLDVTLANGKVDRGAWKYELRNVNGRYDLYNPEVERRNQIVDTTNIATTNDIQADAPSVSSNNEEIARVDEAPVPLPAPPAPATGSAMANEQPETRPAETTQPAMEEANTANSTETVPKSDTATQSDTSNSESVPSETTEKVAENNPQENETVARNEQEAAETTPQNGEVGEVAKEAKPTVEANTQTTETARQPEINSTEETAVKNDLTRSEPKSRPRRSISSSSNNINPAGTEETAKVETEETQKAPQMASQVSPKQAEPVPEKVPTDTNAKEAQPQTQPTTVAAAEATLPNSKPAEETQPNEKTNDEPVTSVSQNQPEKAVSQSTKDKVVVEREEKATVEKEKTQEAPQMASQASPKQEQSETVQPQAELESEKVPTVNNAEAQPQTQTSATVSTEQPAPENSINTGSATAMTETAEKSDKPQTETVASTEDASQHKANTVADNSVANNSASVKPTENSSTKAEQPVTESTTVNTRNSAVENPENTTQPAVNSESSKPKSRHRRSISQPQETSTEETTVTSTEKTTVANNSESSKPNRRSRRSVSQPQETSTEETTVTSTEKTTVADNSESNKTNSRRRSRRSVRSEPTVTSGSNRSAVALRYLTSTNTNAVLSDAMAKAQFVALNVGKAVSQHISQLEMNNEGQYNVWVSNTSMNENYSSSQYRRFSSKSTQTQLGWDQTISNNVQLGGVFTYVRNSNNFDKASSKNTLAQANLYSKYYADNHWYLGIDLGYGKFQSNLQTNHNAKFARHTAQFGLTAGKAFNLGNFGITPIVGVRYSYLSNADFALDQDRIKVNPISVKTAFAQVDLNYTYHLGEFSVTPILSARYDANQGSGKINVNQYDFAYNVENQQQYNAGLKLKYHNVKLSLIGGLTKAKQAEKQKTAELKLSFSF

SEQ ID NO: 7 - protein, 1815 aa, Neisseria meningitidis
MKTKRFKINAISLSIFLAYALTPYSEAALVRDDVDYQIFRDFAENKGKFFVGATDLSVKNKQGQNIGNALSNVPMIDFSVADVNRRTLTVIDPQYAVSVKHVKGDEISYYGHHNGHLDVSNDENEYRSVAQNDYEPNKNWHHGNQGRLEDYNMARLNKFVTEVAPIAPTSAGGGVETYKDKNRFSEFVRVGAGTQFEYNSRYNMTELSRAYRYAIAGTPYQDVNVTSNLNQEGLIGFGDNSKHHSPEKLKEVLSQNALTNYAVLGDSGSPLFAYDKQEKRWVFLGAYDYWAGYQKNSWQEWNIYKKEFADEIKQRDNAGTIKGNGEHHWKTTGTNSHIGSTAVRLANNERDANNGQNVTFENNGTLVLDQNINQGAGGLFFKGDYTVKGINNDITWLGAGIDVADGKKVVWQVKNPNGDRLAKIGKGTLEINGTGVNQGQLKVGDGTVILNQQADADKKVQAFSQVGIVSGRGTLVLNSSNQINPDNLYFGFRGGRLDANGNDLTFEHIRNVDEGARIVNHNTGHASTITLTGKSLITDPKTISIHYIQNNDDDDAGYYYYRPRKPIPQGKDLYFKNYRYYALKSGGSVNAPMPENGQTENNDWILMGSTQEEAKKNAMNHKNNQRISGFSGFFGEENGKGHNGALNLNFNGKSAQNRFLLTGGTNLNGKISVTQGNVLLSGRPTPHARDFVNKSSARKDAHFSKNNEVVFEDDWINRTFKATEIAVNQSASFSSGRNVSDITANITATDNAKVNLGYKNGDEVCVRSDYTGYVTCNTGNLSDKALNSFGATQINGNVNLNQNAALVLGKAALWGQIQGQGNSRVSLNQHSKWHLTGDSQVHNLSLADSHIHLNNASDAQSANKYHTLKINHLSGNGHFHYLTHLAKNLGDKVLVKESASGHYQLHVQDKTGEPNQEGLNLFDASSVQDRSRLSVSLANNHVDLGALRYTIKTENGITRLYNPYAENRRRVKPAPSPATNTASQAQKATQTDGAQIAKPQNIVVAPPSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARRHEKEREAAELSAKQRVGEEERRQTAQSQPQRRKRRAAPQDYMAASQDRPKRRGHRSVQQNNVEIAQAQAELARRQQEERKAAELLAKQRAEAEREAQALAARRKAEAEEAKRQAAELAHRQEAERKAAELSANQKAAAEAQALAARQQKALARQQEEARKAAELAVKQKAETERKTAELAKQRAAAEAAKRQQEARQTAELARRQEAERQAAELSAKQKAETDREAAESAKRKAEEEEHRQAAQSQPQRRKRRAAPQDYMAASQNRPKRRGRRSTLPAPPSPSFDSSAYAAPRALHNPDWYENDYEEIPLDALEDENVSESVDTSDKQPQDNTELHEKYENDYEEIPLDALEDEDVSESVDTSDKQPQDNTELHEKVETVSLQPRAAQPRAQAATQLQAQAAAQADAVSTNTNSALSDAMASTQSILLDTGASLTRHIAQKSRADAEKNSVWMSNTGYGRDYASAQYRRFSSKRTQTQIGIDRSLSENMQIGGVLTYSDSQHTFDQASGKNTFVQANLYGKYYLNDAWYVAGDIGAGSLRSRLQTQQKANFNRASIQTGLTLGNTLKINQFEIVPSAGIRYSRLSSADYKLGNDSVKVSSMSVKTLTAGLDFAYRFKVGNLTVKPLLSAAYFANYGKGGVNVGGNSFVYKADNQQQYSAGAALLYRNVTLNVNGSITKGKQLEKQKSGQIKIQIRF

SEQ ID NO: 8 - protein, 1818 aa, Neisseria meningitidis
MKTKRFKINAISLSIFLAYALTPYSEAALVRDDVDYQIFRDFAENKGKFFVGATDLSVKNKQGQNIGNALSNVPMIDFSVADVNKRIATVVDPQYAVSVKHAKAEVHTFYYGQYNGHNDVADKENEYRVVEQNNYKPHKAWNASNLGRLEDYNMARFNKFVTEVAPIAPTDAGGGLDTYKDKNRFSSFVRVGAGRQLVYEKGAYHPEGKEKGYDLRDLSQAYRYAIAGTPYKDINIDQTMNTEGLIGFGNHNKQYSAEELKQALSQDALTNYGVLGDSGSPLFAFDKQKNQWVFLGTYDYWADYGKKSWQEWNIYKKEFADEIKQRDNAGTIKGYGEHHWKTTGTNSHIGSTAVRLAGNERGANNGQNVTFENNGTLVLDQNINQGAGGLFFKGDYTVKGINNDITWLGAGIDVADGKKVVWQVKNPNGDRLAKIGKGTLEINGTGVNQGQLKVGDGTVILNQKADSNQKVQAFSQVGIVSGRGTLVLNSPNQINPDNLYFGFRGGRLDANGNDLTFEHIRNVDEGARIVNHNTGHTSTITLTGKSLITNPNSLSVHSIQNDYDEDDYSYYYRPRRPIPQGKDLYYKNYRYYALKSGGSVNAPMPENGQTENNDWILMGSTQEEAKKNAMNHKNNQRISGFSGFFGEENGKGHNGALNLNFNGKSAQNRFLLTGGTNLNGKISVTQGNVLLSGRPTPHARDFVNKSSARKDAHFSKNNEVVFEDDWINRTFKATEIAVNQSASFSSGRNVSDITANITATDNAKVNLGYKNGDEVCVRSDYTGYVTCNTDNLSDKALNSFGATQINGNVNLSQNAALTLGKAALWGQIQGQGNSRVSLNQHSKWHLTGDSQVHNLSLADSHIHLNNASDAQSANKYHTLKINHLSGNGHFHYLTHLAKNLGDKVLVKESASGHYQLHVQDKTGEPNQEGLNLFDASSVRDRSHLSVSLANNHVDLGALRYTIKTENGITRLYNPYAENRRRVKPAPSPATNTASQAQTDSAQIAKPQNIVVAPPSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARRHEKEREAAELSAKQRVGEEERRQTAQSQPQRRKRRAAPQDYMAASQDRPKRRGHRSVQQNNVEIAQAQAELARRQQEERKAAELLAKQRAEAEREAQALAARRKAEAEEAKRQAAELAHRQEAERKAAELSANQKAAAEAQALAARQQKALARQQEEARKAAELAVKQKAETERKTAELAKQRAAAEAAKRQQEARQTAELARRQEAERQAAELSAKQKAETDREAAESAKRKAEEEEHRQAAQSQPQRRKRRAAPQDYMAASQNRPKRRGRRSTLPAPPSPSFDSSAYAAPRALHNPDWYENDYEEIPLDALEDENVSESVDTSDKQPQDNTELHEKYENDYEEIPLDALEDEDVSESVDTSDKQPQDNTELHEKVETVSLQPRAAQPRAQAAAQPQAQADAVSTNTNSALSDAMASTQSILLDTGASLTRHIAQKSRADAEKNSVWMSNIGYGRDYASAQYRRFSSKRTQTQIGIDRSLSENMQIGGVLTYSDSQHTFDQASGKNTFVQANLYGKYYLNDAWYVAGDIGAGSLRSRLQTQQKANFNRTSIQTGLTLGNTLKINQFEIVPSAGIRYSRLSSADYKLGNDSVKVSSMSVKTLTAGLDFAYRFKVGNLTVKPLLSAAYFANYGKGGVNVGGNSFAYKADNQQQYSAGAALLYRNVTLNVNGSITKGKQLEKQKSGQIKIQIRF

SEQ ID NO: 9 - protein, 997 aa, Neisseria meningitidis
LAYALTPYSEAALVRDDVDYQIFRDFAENKGKFFVGATDLSVKNKQGQNIGNALSNVPMIDFSVADVNRRTLTVIDPQYAVSVKHVKGDEISYYGHHNGHLDVSNDENEYRSVAQNDYEPNKNWHHGNQGRLEDYNMARLNKFVTEVAPIAPTSAGGGVETYKDKNRFSEFVRVGAGTQFEYNSRYNMTELSRAYRYAIAGTPYQDVNVTSNLNQEGLIGFGDNSKHHSPEKLKEVLSQNALTNYAVLGDSGSPLFAYDKQEKRWVFLGAYDYWAGYQKNSWQEWNIYKKEFADKIKQRDNAGTIKGNGEHHWNITFGTNSKIGSTAVRLAGNEKDANNGQNVTFEDNGTLVLDQNINQGAGGLFFKGDYTVKGINNDITWLGAGIDVTDGKKVVWQVKNPNGDRLAKIGKGTLEINGTGVNQGQLKVGDGTVILNQQADADKKVQAFSQVGIVSGRGTLVLNSSNQINPDNLYFGFRGGRLDANGNDLTFEHIRNVDEGARIVNHNTSHASTITLTGKSLITNPNSLSVHSIQNDYDEDDYSYYYRPRRPIPQGKDLYYKNYRYYALKSGGSVNAPMPENGVTENNDWVFMGYTQEEAKKNAMNHKNNQRISGFSGFFGEENGKGHNGALNLNFNGKSAQNRFLLTGGTNLNGKISVTQGNVLLSGRPTPHARDFVNKSSARKDAHFSKNNEVVFEDDWINRTFKAAEIAVNQSASFSSGRNVSNITANITATDNAKVNLGYKNGDEVCVRSDYTGYVTCNTGNLSDKALNSFGATQINGNVNLNQNAALVLGKAALWGQIQGQGNSRVSLNQHSKWHLTGDSQVHNLSLADSHIHLNNASDAQSANQYHTLKINHLSGNGHFHYLTHLAENLGDKVLVKESASGHYQLHVQDKTGEPNQEGLNLFDASSVQDRSRLSVSLANNHVDLGALRYTIKTENGITRLYNPYAENRRRVKPAPSPATNTASQAQKATQTDGAQIAKPQNIVVAPPSPQAN

SEQ ID NO: 10 - protein, 1593 aa, Neisseria gonorrhoeae
MKAKRFKINAISLSIFLAYALTPYSEAALVRDDVDYQIFRDFAENKGKFFVGATDLSVKNKRGQNIGNALSNVPMIDFSVADVNKRIATVVDPQYAVSVKHAKAEVHTFYYGQYNGHNDVADKENEYRVVEQNNYEPHKAWSASNLGRLEDYNMARFNKFVTEVAPIAPTDAGGGLDTYKDKNRFSSFVRIGAGRQLVYEKGVYHQEGNEKGYDLRDLSQAYRYAIAGTPYKDINIDQTMNTEGLIGFGNHNKQYSAEELKQALSQDALTNYGVLGDSGSPLFAFDKQKNQWVFLGTYDYWAGYGKKSWQEWNIYKKEFADKIKQHDNAGTVKGNGEHHWKTTGTNSHIGSTAVRLANNEGDANNGQNVTFEDNGTLVLDQNINQGAGGLFFKGDYTVKGANNDITWLGAGIDVADGKKVVWQVKNPNGDRLAKIGKGTLEINGTGVNQGQLKVGDGTVILNQKADSNQKVQAFSQVGIVSGRGTLVLNSPDQINPNNLYFGFRGGRLDANGNDLTFEHIRNVDEGARIVNHNTDHASTITLTGKSLITNPNSLSVHSIQNDYDEDNYSYYYRPRRPIPQGKDLYYKNYRYYALKSGGSVNAPMPENGQTENNDWILMGSTQEEAKKNAMNHKNNQRISGFSGFFGEENGKGHNGALNLNFNGKSAQNRFLLTGGANLNGKISVTQGNVLLSGRPTPHARDFVNKSSARKDAHFSKNNEVVFEDDWINRTFKAAEIAVNQSASFSSGRNVSDITANITATDNAKVNLGYKNGDEVCVRSDYTGYVTCNTGNLSDKALNSFDATRINGNVNLNQNAALVLGKAALWGQIQGQGNSRVSLNQHSKWHLTGDSQVHNLSLADSHIHLNNASDAQSANKYHTIKINHLSGNGHFHYLTDLAKNLGDKVLVKESASGHYQLHVQNKTGEPNQEGLDLFDASSVQDRSRLFVSLANHYVDLGALRYTIKTENGITRLYNPYAGNRRPVKPAPSPAANTASQAQKATQTDGAQIAKPQDIVVAPPSPQANQAEEAKRQQAKAEQVKRQQAEAGRKSAELSAKQRAGEEERRQTAQSQPQRRKRRAAPQDYMAVSQDRPKRRGHRSVQQNNVEIAQAQAELARRQQEERKAAELLAKQRAEAEREAQALAARRKAEAEEAKHQAAELAHRQEAKRKAAESAKRKAEEEEHRQTAQSQPQRRKRRAAPQDYMAVSQDRPKRRGRRSTLPAPPSPSFDSSAYAAPRALHNPDWYENDYEEIPLDALEDEDVSESVDTSDKQPQDNTELHEKVEAVSLQPRAAQPRTQAAAQADAVSTNTNSALSDAMASTQSILLDTGASLTRHIAQKSRADAEKNSVWMSNTGYGRDYASAQYRRFSSKRTQTQIGIDRSLSENMQIGGVLTYSDSQHTFDQAGGKNTFVQANLYGKYYLNDAWYVAGDIGAGSLRSRLQTQQKANFNRTSIQTGLTLGNTLKINQFEIVPSAGIRYSRLSSADYKLGDDSVKVSSMAVKTLTAGLDFAYRFKVGNLTVKPLLSAAYFANYGKGGVNVGGKSFAYKADNQQQYSAGAALLYRNVTLNVNGSITKGKQLEKQKSGQIKIQIRF

SEQ ID NO: 11 - protein, 1532 aa, Neisseria gonorrhoeae
MKAKRFKINAISLSIFLAYALTPYSEAALVRDDVDYQIFRDFAENKGKFFVGATDLSVKNKRGQNIGNALSNVPMIDFSVADVNKRIATVVDPQYAVSVKHAKAEVHTFYYGQYNGHNDVADKENEYRVVEQNNYEPHKAWGASNLGRLEDYNMARFNKFVTEVAPIAPTDAGGGLDTYKDKNRFSSFVRIGAGRQLVYEKGVYHQEGNEKGYDLRDLSQAYRYAIAGTPYKDINIDQTMNTEGLIGFGNHNKQYSAEELKQALSQDALTNYGVLGDSGSPLFAFDKQKNQWVFLGTYDYWAGYGKKSWQEWNIYKKEFADKIKQHDNAGTVKGNGEHHWKTTGTNSHIGSTAVRLANNEGDANNGQNVTFEDNGTLVLNQNINQGAGGLFFKGDYTVKGANNDITWLGAGIDVADGKKVVWQVKNPNGDRLAKIGKGTLEINGTGVNQGQLKVGDGTVILNQKADADKKVQAFSQVGIVSGRGTLVLNSSNQINPDNLYFGFRGGRLDANGNDLTFEHIRNVDEGARIVNHNTDHASTITLTGKSLITNPNSLSVHSIQNDYDEDDYSYYYRPRRPIPQGKDLYYKNYRYYALKSGGRLNAPMPENGVAENNDWIFMGYTQEEARKNAMNHKNNRRIGDFGGFFDEENGKGHNGALNLNFNGKSAQNRFLLTGGANLNGKISVTQGNVLLSGRPTPHARDFVNKSSARKDAHFSKNNEVVFEDDWINRTFKAAEIAVNQSASFSSGRNVSDITANITATDNAKVNLGYKNGDEVCVRSDYTGYVTCNTGNLSDKALNSFDATRINGNVNLNQNAALVLGKAALWGKIQGQGNSRVSLNQHSKWHLTGDSQVHNLSLADSHIHLNNASDAQSANKYHTIKINHLSGNGHFHYLTDLAKNLGDKVLVKESASGHYQLHVQNKTGEPNQEGLDLFDASSVQDRSRLFVSLANHYVDLGALRYTIKTENGITRLYNPYAGNGRPVKPAPSPAANTASQAQKATQTDGAQIAKPQNIVVAPPSPQANQAEEALRQQAKAEQVKRQQAAEAEKVARQKDEEAKRKAAEIARQQEEARKAAELAAKQKAEAERKARELARQKAEEASHQANAKPKRRRRRAILPRPPAPVFSLDDYDAKDNSESSIGNLARVIPRMGRELINDYEEIPLEELEDEAEEERRQATQFHSKSRNRRAISSEPSSDEDASESVSTSDKHPQDNTELHEKVETAGLQPRAAQPRTQAAAQADAVSTNTNSALSDAMASTQSILLDTGAYLTRHIAQKSRADAEKNSVWMSNTGYGRDYASAQYRRFSSKRTQTQIGIDRSLSENMQIGGVLTYSDSQHTFDQAGGKNTFVQANLYGKYYLNDAWYVAGDIGAGSLRSRLQTQQKANFNRTSIQTGLTLGNTLKINQFEIVPSAGIRYSRLSSADYKLGDDSVKVSSMAVKTLTAGLDFAYRFKVGNLTVKPLLSAAYFANYGKGGVNVGGKSFAYKADNQQQYSAGVALLYRNVTLNVNGSITKGKQLEKQKSGQIKIQIRF

SEQ ID NO: 12 - protein, 1532 aa, Neisseria gonorrhoeae
MKAKRFKINAISLSIFLAYALTPYSEAALVRDDVDYQIFRDFAENKGKFFVGATDLSVKNKRGQNIGNALSNVPMIDFSVADVNKRIATVVDPQYAVSVKHAKAEVHTFYYGQYNGHNDVADKENEYRVVEQNNYEPHKAWGASNLGRLEDYNMARFNKFVTEVAPIAPTDAGGGLDTYKDKNRFSSFVRIGAGRQLVYEKGVYHQEGNEKGYDLRDLSQAYRYAIAGTPYKDINIDQTMNTEGLIGFGNHNKQYSAEELKQALSQDALTNYGVLGDSGSPLFAFDKQKNQWVFLGTYDYWAGYGKKSWQEWNIYKKEFADKIKQHDNAGTVKGNGEHHWKTTGTNSHIGSTAVRLANNEGDANNGQNVTFEDNGTLVLNQNINQGAGGLFFKGDYTVKGANNDITWLGAGIDVADGKKVVWQVKNPNGDRLAKIGKGTLEINGTGVNQGQLKVGDGTVILNQKADADKKVQAFSQVGIVSGRGTLVLNSSNQINPDNLYFGFRGGRLDANGNDLTFEHIRNVDEGARIVNHNTDHASTITLTGKSLITNPNSLSVHSIQNDYDEDDYSYYYRPRRPIPQGKDLYYKNYRYYALKSGGRLNAPMPENGVAENNDWIFMGYTQEEARKNAMNHKNNRRIGDFGGFFDEENGKGHNGALNLNFNGKSAQKRFLLTGGANLNGKISVTQGNVLLSGRPTPHARDFVNKSSARKDAHFSKNNEVVFEDDWINRTFKAAEIAVNQSASFSSGRNVSDITANITATDNAKVNLGYKNGDEVCVRSDYTGYVTCNTGNLSDKALNSFDATRINGNVNLNQNAALVLGKAALWGKIQGQGNSRVSLNQHSKWHLTGDSQVHNLSLADSHIHLNNASDAQSANKYHTIKINHLSGNGHFHYLTDLAKNLGDKVLVKESASGHYQLHVQNKTGEPNQEGLDLFDASSVQDRSRLFVSLANHYVDLGALRYTIKTENGITRLYNPYAGNGRPVKPAPSPAANTASQAQKATQTDGAQIAKPQNIVVAPPSPQANQAEEALRQQAKAEQVKRQQAAEAEKVARQKDEEAKRKAAEIARQQEEARKAAELAAKQKAEAERKARELARQKAEEASHQANAKPKRRRRRAILPRPPAPVFSLDDYDAKDNSESSIGNLARVIPRMGRELINDYEEIPLEELEDEAEEERRQATQFHSKSRNRRAISSEPSSDEDASESVSTSDKHPQDNTELHEKVETAGLQPRAAQPRTQAAAQADAVSTNTNSALSDAMASTQSILLDTGAYLTRHIAQKSRADAEKNSVWMSNTGYGRDYASAQYRRFSSKRTQTQIGIDRSLSENMQIGGVLTYSDSQHTFDQAGGKNTFVQANLYGKYYLNDAWYVAGDIGAGSLRSRLQTQQKANFNRTSIQTGLTLGNTLKINQFEIVPSAGIRYSRLSSADYKLGDDSVKVSSMAVKTLTAGLDFAYRFKVGNLTVKPLLSAAYFANYGKGGVNVGGKSFAYKADNQQQYSAGVALLYRNVTLNVNGSITKGKQLEKQKSGQIKIQIRF

SEQ ID NO: 13 - DNA, 30 bp, Artificial Sequence
  Synthetic primer
gctcatatgctaaataaaaaattcaaactc

SEQ ID NO: 14 - DNA, 56 bp, Artificial Sequence
  Synthetic primer
caaggatcctaggtggtggtggtggtggtgaggcacatcagcttgaatattattag

SEQ ID NO: 15 - DNA, 30 bp, Artificial Sequence
  Synthetic primer
gctcatatggcgttagtgagagacgatgtg

SEQ ID NO: 16 - DNA, 29 bp, Artificial Sequence
  Synthetic primer
caaggatcctaggtggtggtggtggtggt

SEQ ID NO: 17 - DNA, 27 bp, Artificial Sequence
  Synthetic primer
gaggcacatcagcttgaatattattag

SEQ ID NO: 18 - DNA, 30 bp, Artificial Sequence; identical to SEQ ID NO: 13
  Synthetic primer

SEQ ID NO: 19 - DNA, 56 bp, Artificial Sequence; identical to SEQ ID NO: 14
  Synthetic primer

SEQ ID NO: 20 - DNA, 30 bp, Artificial Sequence; identical to SEQ ID NO: 15
  Synthetic primer

SEQ ID NO: 21 - DNA, 56 bp, Artificial Sequence; identical to SEQ ID NO: 14
  Synthetic primer

SEQ ID NO: 22 - protein, 1694 aa, Haemophilus influenzae; identical to SEQ ID NO: 5

SEQ ID NO: 23 - DNA, 5657 bp, Haemophilus influenzae
tttttaaaaattattatcattacctcataaatgtaattcaagttttgagcgatttatgctataaagctcgctcattataaagatgaagacaacttgcaaattatcaacgcaacacagccaaaatttgaatcaacttgtaaccgtatatcaaattgtgtcctatcaaatctactttttaaacttaattaataaggacagcttctatgctaaataaaaaattcaaactcaattttattgcgcttactgtcgcctacgcattaaccccttatacagaagctgcgttagtgagagacgatgtggattatcaaatatttcgtgattttgcagaaaataaagggagattttctgttggtgcaacaaatgtggaagtgagagataaaaataaccactctttaggcaatgttttacctaatggcattccgatgattgattttagtgttgtggatgtagataaacgcatcgccacattgataaatccacaatatgtagtaggtgtaaaacacgttagtaacggcgtgagtgaactacattttgggaacttaaatggcaatatgaataatggcaatgctaaatcgcaccgagatgtatcttcagaagaaaatagatatttttccgttgagaaaaatgagtatccaactaaattgaatggaaaagcagtaactactgaagatcaaactcaaaaacgccgtgaagactactatatgccacgtcttgataaatttgttaccgaagttgcaccaatagaggcttcaactgcaagtagtgatgctggcacatataatgatcagaataaatatcctgcttttgtaagactaggaagtggtagtcaatttatttataaaaaaggagataattacagcttaattttaaataatcatgaggttggaggcaataatcttaaattggtgggcgatgcctatacctatggtattgcaggcacaccttataaagtaaaccacggggttaatggactaattggttttggcaattcaaaagaggaacacagcgatccaaaagccatattatctcaagatccgcttaccaattatgctgttttaggcgacagtggctccccattatttgtatatgatagagaaaaaggaaaatggctttttcttgggtcttatgatttttgggcaggttataacaaaaaatcttggcaagaatggaatatttataaacctgaatttgcaaaaactgttctagataaagatactgcaggttctttaactggttctaacacccaatacaattggaatcctactggcaaaacaagcgttatttctaatggttctgaatctctaaatgttgatttattcgatagtagtcaggatacggactctaagaagaacaatcacggaaaaagtgtgactcttagaggaagtggaacgcttaccttaaataataatatcgatcaaggcgcaggcggcttgttctttgaaggagattatgaagttaaaggcacttctgatagtaccacttggaaaggagctggcgtttctgttgctgatggaaaaacagtaacgtggaaagtacataacccgaaatctgatcgtttagctaaaatcggcaaaggaacattaattgtagaagaaaagggagaaaataaaggttcgctaaaagtgggcgatggtactgttatcttaaaacaacaagctgatgccaataataaagttaaagccttttcacaagtaggtatagtaagtggtcgctcaactgttgtacttaatgatgataagcaagtagatccaaattccatttactttggctttagaggtggtcgattagatgccaatggcaataatctcacttttgaacatatccgtaatattgatgatggcgcaagactagtaaatcacaataccagcaaaacctctactgtaacaattactggggaaagtctaattacagatccaaatacaattactccatataatatagacgcaccagatgaagataatccttatgcctttcgacggattaaagatggaggacagctctatttaaatttggaaaattacacttattatgcgttaagaaaaggtgcgagcactcgttcagaattacctaaaaatagtggcgaaagcaatgaaaattggctatatatgggtaaaacttccgatgaagccaaaagaaatgtaatgaaccatatcaacaacgagcgtatgaatggctttaacggttattttggcgaggaagagggtaaaaataacggtaatctaaatgtgacttttaaaggcaaaagtgagcaaaatcgctttttattaacaggcggaacaaaccttaatggcgatttaaaggttgaaaaaggcacattattcctttctggcagaccaacaccgcacgcaagagatattgcaggtatttcttcgacaaaaaaagatcaacactttgctgaaaataatgaagtggtagtagaagatgactggattaaccgcaattttaaagcaacaaatattaatgtaaccaataacgcaaccctttattcaggtcgcaatgttgcaaacattacttcaaatatcacagcttctgataatgcaaaagtacatattggctataaagcaggcgataccgtttgtgtacgttctgactatacgggctatgtgacttgcactactgacaagttatccgataaagcccttaatagctttaacgccaccaatgtatctggcaatgtaaatttatcaggtaatgcaaactttgtcttaggcaaagctaacttattcggcacaattagcggcacgggaaatagccaagtacgtttaaccgaaaatagccattggcatttaacaggcgatacgaatgttaatcagttaaatttagacaaggggcatattcatttaaatgcacaaaacgatgcaaataaagtaactacatataacacgctgactgtgaatagcttatcaggtaacggttctttctattatttaactgatctttccaataaacaaggcgacaaagttgttgtaactaaatccgccacaggtaactttacattacaagtggcagataaaacaggcgagcctacaaaaaatgaactcacgctttttgatgcgtcaaatgctacaagaaataatttgaatgtgtcattagttgggaataccgttgatttaggtgcttggaaatataaattacgtaatgttaatggacgttacgatttgtataacccagaggtggaaaaaagaaatcaaactgtcgatacgacaaatatcacaacacctaataatattcaagctgatgtgcctagcgtaccaagtaacaatgaagaaatagcccgtgttgaaacaccagttccaccacctgcgcctgatacaccatcagagacaactgaaacagtggctgaaaatagtaagcaagaaagtaaaacagtagagaaaaacgagcaagacgcaaccgagacaacagctcaaaatggagaagttggagaagaagctaaaccaagtgtaaaagctaatactcaaacaaatgaagtggctcaaagtggaagtgaaaccgaggaaactcaaacgactgaaataaaagaaacagctaaagtagaaaaagaggaaaaggctaaagtagaaaaagatgaaattcaagaagcacctcaaatggcttctgaaacgtctccgaaacaagcaaagcctgctcctaaagaagtttcaactgatacgaaagtagaagaaactcaagttcaagctcaaccgcaaacacaatcgacaactgttgctgcggcagaggcaacttcgccaaacagtaaaccagcggaagaaactcaaccaagtgaaaaaactaacgctgaacctgtaacgcctgtagtatcaaaaaatcaaacagaaaatacgaccgaccaaccaacagaaagagagaaaacggctaaagtagaaacagagaaaactcaagaaccccctcaagtggcttctcaagcgtctccgaaacaggaacagtctgaaactgttcaaccgcaagcagtgcttgaaagtgaaaatgttccgactgttaataatgcagaagaagttcaagctcaactgcaaacacaaacaagtgcaacagtaagcactaaacaacctgcaccagagaattcaataaatactggatctgcaaccgcaataacagaaactgctgaaaaatccgataaaccacaaacggaaactgcggcttcgactgaagatgctagtcagcataaagcgaatactgttgcggataattctgtagcaaataattcagaaagcagtgatccaaagagtagacgtagaagaagtattagccagccgcaagagacttctgctgaagaaacaacagcagcttctactgacgaaacaacaatagctgataattcaaaacgcagtaagccaaatcgtagaagtagaagaagtgttcgctcggaaccaactgttacaaatggcagcgatcgttctacagtagcattgcgcgatctcacaagtacaaacacaaatgcggtaatttctgatgcaatggcaaaaggacaatttgttgcattaaatgtggggaaagcagtttctcaacatattagccagttagaaatgaataacgaggggcaatataacgtttgggtatctaatacttcaatgaacgaaaattattcctcaagtcaatatcgtcgttttagttctaaaagtacgcaaactcaacttggttgggatcaaacaatctcaaacaatgttcagttaggtggcgtgtttacttatgttcgcaatagtaacaactttgataaggcaagcagtaaaaatactctagcacaagttaatttctattctaaatattatgcggataatcattggtatttgggcattgatttaggctacggcaagttccaaagcaacctaaaaaccaatactaatgcgaaatttgctcgccatactgcacaatttggtttaaccgcaggcaaagcatttaatcttggcaattttggtattacgccaatagtaggcgtgcgttatagctatttatcaaacgctaattttgcattagctaaagatcgcattaaagtaaatccaatatctgtcaaaacagcctttgctcaagttgatttaagttatacttatcacttaggcgagttttccgttacgccaattttgtctgctcgatatgatacaaatcaaggcagcggaaaaattaatgtaaatcaatatgattttgcttacaacgtggaaaaccaacagcaatataacgcagggcttaaattgaaatatcataatgtgaaattaagtctaataggcggattaacaaaagcgaaacaagcggaaaaacaaaaaactgcagaattaaaactaagttttagtttttaataagcctgtttgaattaacgttataaacaacaaagccctgtgtcttacagggctttatttttgaatgaaattcagtgattaagtgcggtgaaaaatcagcgcattttttatttttaacgtaaaaacgctggaatatttttctcgtatgctgagattttgtcttcgtgctgaagagttaagccgatattatctaaaccgtttagcaaacaatggcggcggaattcatcaagctcaaaagtataaactttatcccctacagtgaccgagatcgcttctaaatctacgtggatttgtttgccttcatttgcccatacccattggaagatttcttctacttcttcttcgcttaaacgaatcggtaacatatgc

SEQ ID NO: 24 - DNA, 37 bp, Artificial Sequence
  Synthetic primer
caaggatcctaaggcacatcagcttgaatattattag